(19) United States
(12) Patent Application Publication
(10) Pub. No.: US 2025/0319308 A1
(43) Pub. Date: Oct. 16, 2025

(54) Tumor Electric Field Treatment System

(71) Applicant: JIANGSU HEALTHY LIFE INNOVATION MEDICAL TECHNOLOGY CO., LTD., Jiangsu (CN)

(72) Inventors: Qichao SHEN, Jiangsu (CN); Jianjun YING, Jiangsu (CN); Jing YU, Jiangsu (CN); Jun ZHANG, Jiangsu (CN); Sheng CHEN, Jiangsu (CN); Hu SUN, Jiangsu (CN); Jiajie HUI, Jiangsu (CN); Yidong SUN, Jiangsu (CN)

(21) Appl. No.: 18/696,666

(22) PCT Filed: Nov. 28, 2022
(86) PCT No.: PCT/CN2022/134717

§ 371 Date: Oct. 14, 2024

(30) Foreign Application Priority Data
Sep. 28, 2021 (CN) 202111143956.3
Dec. 22, 2021 (CN) 202111578521.1
Dec. 22, 2021 (CN) 202111578531.5
Dec. 22, 2021 (CN) 202111578597.4
Dec. 22, 2021 (CN) 202111580039.1
Dec. 22, 2021 (CN) 202111580040.4
Dec. 22, 2021 (CN) 202111580105.5
Dec. 22, 2021 (CN) 202111580121.4
Dec. 22, 2021 (CN) 202111580130.3
Dec. 22, 2021 (CN) 202111580142.6
Dec. 22, 2021 (CN) 202111580196.2
Dec. 22, 2021 (CN) 202123242599.4
Dec. 31, 2021 (CN) 202111667246.0

(51) Int. Cl. A61N 1/36; A61N 1/02

(52) U.S. Cl. CPC A61N 1/36002; A61N 1/025; A61N 1/36034

(57) ABSTRACT

A system includes an electric field therapy device and two pairs of insulated electrodes electrically connected to the device. The device includes an MCU control unit, a direction control unit, a DC power control unit, and an AC voltage control unit. The control unit is configured to control the AC voltage control unit to generate an alternating electrical signal with an AC voltage. The control unit is further configured to drive the direction control unit to apply the alternating electrical signals received by the AC voltage control unit to different pairs of insulated electrodes, to achieve the switching of the alternating electrical signal between the pairs of insulated electrodes. The control unit is further configured to, when the alternating electrical signal is being switched, control the DC power control unit to increase or decrease the AC voltage in a manner that the voltage change per unit time is constant.

### CROSS-REFERENCE TO RELATED APPLICATIONS

This application is a US national application of International application No. PCT/CN2022/134717 field on Nov. 28, 2022, which claims the priority of the following patent applications: Chinese Patent Application No. 202111578597.4 filed on Dec. 22, 2021, Chinese Patent Application No. 202111667246.0 filed on Dec. 31, 2021, Chinese Patent Application No. 202111580105.5 filed on Dec. 22, 2021, Chinese Patent Application No. 202111580121.4 filed on Dec. 22, 2021, Chinese Patent Application No. 202111580130.3 filed on Dec. 22, 2021, Chinese Patent Application No. 202111580142.6 filed on Dec. 22, 2021, Chinese Patent Application No. 202111578521.1 filed on Dec. 22, 2021, Chinese Patent Application No. 202111580040.4 filed on Dec. 22, 2021, Chinese Patent Application No. 202111580039.1 filed on Dec. 22, 2021, Chinese Patent Application No. 202123242599.4 filed on Dec. 22, 2021, Chinese Patent Application No. 202111578531.5 filed on Dec. 22, 2021, Chinese Patent Application No. 202111580196.2 filed on Dec. 22, 2021, and a Chinese Patent Application No. 202111143956.3 filed on Sep. 28, 2021, the entire content of the above-mentioned Chinese patent applications are incorporated in this application by reference.

### TECHNICAL FIELD

The application relates to a medical device, more particularly relates to a tumor electric field therapy system or a tumor-treating fields system (called TTFields system for short).

### BACKGROUND

A tumor electric field therapy system and an electric field application method thereof are disclosed in a Chinese Invention Patent issued as CN104771830B. The TTFields system includes an electric field treatment device generating an alternating electrical signal with alternating voltage and two pairs of insulated electrodes electrically connected to the electric field treatment device. Two pairs of insulated electrodes are arranged perpendicular to each other around malignant tumor sites in experimental animals or around proliferating cells in tissue culture and periodically and alternately apply the alternating electrical signal generated by the electric field treatment device to the malignant tumor sites in the experimental animals or the proliferating cells in the tissue culture to treat the malignant tumors in the experimental animals or inhibit the proliferating cells in the tissue culture. The electric field treatment device includes an Alternating Current (AC) signal generator and an AC signal controller electrically connected to the AC signal generator. The AC signal controller generates a periodic control signal with two output states to control the AC signal generator to generate alternately applied alternating signals between two pairs of insulated electrodes. Specifically, when the AC signal controller is in the first output state, the AC signal controller controls the AC signal generator to generate an alternating signal between the first pair of insulated electrodes but no alternating signal is generated between the second pair of insulated electrodes; and when the AC signal controller is in the second output state, the AC signal controller controls the AC signal generator to generate an alternating signal between the second pair of insulated electrodes but no alternating signal is generated between the first pair of insulated electrodes. That is, when the alternating electric signal between the first pair of insulated electrodes is conducted, the alternating electric signal between the second pair of insulated electrodes is interrupted; and when the alternating electric signal between the first pair of insulated electrodes is interrupted, the alternating electric signal between the second pair of insulated electrodes is conducted.

The aforementioned tumor electric field therapy system achieves the switching of the alternating electric signal generated by the AC signal generator and applied between two pairs of insulated electrodes by controlling the switching of the AC signal controller between the first and second output states, thereby alternately applying alternating electric fields in different directions to the malignant tumor sites of experimental animals or proliferating cells in tissue cultures to treat tumors or inhibit the proliferating cells. Although the tumor electric field therapy system uses the AC signal controller to alternately apply the alternating electric fields to the malignant tumor sites of the experimental animals or the proliferating cells in the tissue culture through the two pairs of insulated electrodes to achieve the purpose of treating tumors or inhibiting the proliferating cells, a sharp voltage change occurs between the two pairs of insulated electrodes when the alternating voltage of the alternating electric signal switches from zero to a certain value or vice versa. This large voltage fluctuation within a unit of time causes the AC signal controller to generate a spike signal which can impact and potentially damage the electronic components within the AC signal controller. Furthermore, the sharp voltage change of the AC signal may also cause tingling and uncomfortable in scalp of a patient during using the tumor electric field therapy system on the body of the patient.

Therefore, it is certainly necessary to provide a tumor electric field therapy system that can better inhibit the proliferation of tumor cells.

### SUMMARY

The present disclosure provides a tumor electric field therapy system to avoid impact or damage of an electronic element due to a spike signal generated when an alternating electric signal is switched.

The tumor electric field therapy system of the present disclosure can be realized through the following technical solutions. The tumor electric field therapy system includes an electric field therapy device having a plurality of preset system parameters and at least two pairs of insulated electrodes electrically connected to the electric field therapy device. The electric field therapy device includes a MCU control unit with a reference voltage, a direction control unit connected to the MCU control unit, a DC power control unit electrically connected with the MCU control unit, and an AC voltage control unit both electrically connected with the direction control unit and the MCU control unit. The MCU control unit is configured to control the AC voltage control unit to generate an alternating electrical signal with an AC voltage according to the system parameters of the electric field therapy device. According to the system parameters of the electric field therapy device, the MCU control unit is also configured to drive the direction control unit to cyclically and alternately apply the alternating electrical signal received by the AC voltage control unit to different pairs of insulated electrodes, so as to realize the switching of the alternating electrical signal between different pairs of insulated electrodes. The system parameters of the electric field therapy device include an output AC voltage amplitude. The alternating electric signal applied to each pair of insulated electrodes has respective continuous on-time period during each respective working cycles. Each of the continuous on-time period includes an initial switching-on time period t3, an intermediate on-time period and a final switching-off time period t4. The alternating electric signal has a specific voltage during the intermediate on-time period and the specific voltage is equal to or less than a peak value of the output AC voltage amplitude of the electric field therapy device. During the switching-on time period 13, the MCU control unit controls the DC power control unit to increase the AC voltage value of the alternating electric signal applied from the AC voltage control unit to the insulated electrodes arranged in pairs from 0 to a specific voltage at a constant speed, and a voltage change of the alternating electric signal applied by the AC voltage control unit per millisecond is set to be a constant value. During the switching-off time period t4, the MCU control unit controls the DC power control unit to decrease the AC voltage value of the alternating electric signal applied from the AC voltage control unit to the insulated electrodes arranged in pairs from a specific voltage to 0 at a constant speed, and the voltage change of the alternating electric signal applied by the AC voltage control unit per millisecond is set to be a constant value.

According to another aspect of the embodiment of the present disclosure, the ratio of the voltage change per millisecond of the alternating electric signal applied by the AC voltage control unit to the specific voltage is less than 5%.

According to another aspect of the embodiment of the present disclosure, the electric field therapy device further includes an inverter boost control unit both connected to the MCU control unit and the DC power control unit and a filter control unit connected to the inverter boost control unit, and the system parameters of the electric field therapy device include an electric field frequency and a direction switching period of the alternating electrical signal, and the MCU control unit generates a pulse signal transmitted to the inverter boost control unit according to the reference voltage, the electric field frequency of the alternating electrical signal stored in the electric field therapy device, and the output AC voltage amplitude of the alternating electrical signal stored in the electric field therapy device.

According to another aspect of the embodiment of the present disclosure, the MCU control unit drives the direction control unit to cyclically and alternately switch the alternating electrical signal applied to different pairs of insulated electrodes through the AC voltage control unit according to the direction switching period of the alternating electrical signal of the electric field therapy device.

According to another aspect of the embodiment of the present disclosure, the connection and disconnection between the MCU control unit and the DC power control unit and whether the pulse signal is applied to the inverter boost control unit or not are controlled by the MCU control unit according to the direction switching period of the alternating electrical signal of the electric field therapy device.

According to another aspect of the embodiment of the present disclosure, the direction control unit is configured to be switched after the communication between the MCU control unit and the DC power control unit is disconnected and the pulse signal transmitted from the MCU control unit to the inverter boost control unit is stopped.

According to another aspect of the embodiment of the present disclosure, after the direction control unit is switched, the MCU control unit activates the DC power control unit, controls the DC power control unit to output a DC signal to the inverter boost control unit and also outputs a pulse signal to the inverter boost control unit.

According to another aspect of the embodiment of the present disclosure, the AC voltage of the alternating electrical signal is increased from 0 to the specific voltage at a constant speed with a voltage change of 4V per millisecond during the switching-on time period t3.

According to another aspect of the embodiment of the present disclosure, the switching-on time period t3 is determined by the following method: t3=V/ΔV*t, wherein V is the specific voltage, t is 1 ms, and ΔV is the voltage change of the alternating electrical signal outputted by the AC voltage control unit per millisecond.

According to another aspect of the embodiment of the present disclosure, the MCU control unit includes a digital-to-analog conversion module with a DAC data register, and the MCU control unit calculates a voltage output increment of the digital-to-analog conversion module per millisecond based on the reference voltage, the voltage change per millisecond of the alternating electrical signal outputted by the AC voltage control unit, the specific voltage, and the DAC data register value corresponding to the specific voltage.

According to another aspect of the embodiment of the present disclosure, the MCU control unit calculates a voltage output decrement of the digital-to-analog conversion module per millisecond based on the reference voltage, the voltage change per millisecond of the alternating electrical signal outputted by the AC voltage control unit, the specific voltage, and the DAC data register value corresponding to the specific voltage.

According to another aspect of the embodiment of the present disclosure, the voltage output increment or voltage output decrement of the digital-to-analog conversion module per millisecond is determined by the following method: ΔV_{DAC}=(3.3*1000*ΔV*DAC)/(4096*V), wherein ΔV_{DAC} is the voltage output increment or voltage output decrement of the digital-to-analog conversion module per millisecond and measured in millivolts; the reference voltage of the MCU control unit is 3.3V, and the DAC data register value corresponding to the reference voltage is 4096 and equal to 2¹²; ΔV is the voltage change per millisecond of the alternating electrical signal outputted by the AC voltage control unit and measured in volts; V is the specific voltage and measured in volts; DAC is the DAC data register value of the specific voltage in the DAC data register.

According to another aspect of the embodiment of the present disclosure, the switching-off time period t4 is obtained by the following method: t4=V/ΔV*t, wherein Vis a specific voltage, t is 1 ms, and ΔV is the voltage change of the alternating electrical signal outputted by the AC voltage control unit per millisecond.

According to another aspect of the embodiment of the present disclosure, the AC voltage of the alternating electrical signal is decreased from the specific voltage to 0 at a constant speed with a voltage change of 4V per millisecond during the switching-off time period t4.

According to another aspect of the embodiment of the present disclosure, the electric field therapy device further includes a first direction switch that is electrically connected to the direction control unit and controls the connection and disconnection between the AC voltage control unit and one pair of the insulated electrodes, and a second direction switch that is electrically connected with the direction control unit and controls the connection and disconnection between the AC voltage control unit and another pair of the insulated electrodes.

According to another aspect of the embodiment of the present disclosure, the MCU control unit includes a storage module, an execution module communicating with the storage module, a digital-to-analog conversion (DAC) module communicating with the execution module, and a control module controlling the storage module, the execution module and the DAC module to perform corresponding operations; and the storage module of the MCU control unit, the execution module of the MCU control unit, the digital-to-analog conversion module of the MCU control unit, the control module of the MCU control unit, the DC power control unit, the inverter boost control unit and the AC voltage control unit jointly form an AC signal generator; and the storage module of the MCU control unit, the execution module of the MCU control unit, the control module of the MCU control unit, the direction control unit, the first and second direction switches both electrically connected with the direction control unit jointly form an AC signal controller.

According to another aspect of the embodiment of the present disclosure, at least two pairs of the insulated electrodes include a first pair of insulated electrodes and a second pair of insulated electrodes disposed on the patient's torso surface; the AC signal controller is configured to generate periodic control signals each having a first output state with a duration between 500 ms and 980 ms and a second output state with a duration between 500 ms and 980 ms; the AC signal generator generates a first AC signal applied to the first pair of insulated electrodes when the control signal is in the first output state and a second AC signal applied to the second pair of insulated electrodes when the control signal is in the second output state, and the switch between the first AC signal generated between the first pair of insulated electrodes and the second AC signal generated between the second pair of insulated electrodes is achieved by the switch between the first output state and the second output state.

According to another aspect of the embodiment of the present disclosure, the first output state has a duration with a first time period T1, the second output state has a duration with a second time period T2, and the first time period T1 and the second time period T2 are same with each other.

According to another aspect of the embodiment of the present disclosure, the first time period T1 and the second time period T2 are both 50% of respective operating period.

According to another aspect of the embodiment of the present disclosure, the first AC signal has an increasing AC voltage amplitude during the switching-on time period t3 and a decreasing AC voltage amplitude during the switching-off time period t4 in each of the first time period T1; the second AC signal has a increasing amplitude during the switching-on time period t3 and a decreasing amplitude during the switching-off time period t4 in each of the second time period T2.

According to another aspect of the embodiment of the present disclosure, the durations of the switching-on time period t3 and the switching-off time period t4 are both less than 10% of the duration of the first time period T1 or the second time period T2.

According to another aspect of the embodiment of the present disclosure, the first AC signal is applied to the first pair of insulated electrodes to generate a first electric field between the first pair of insulated electrodes, and the second AC signal is applied to the second pair of insulated electrodes to generate a second electric field between the second pair of insulated electrodes.

According to another aspect of the embodiment of the present disclosure, the direction of the first electric field is perpendicular to the direction of the second electric field.

According to another aspect of the embodiment of the present disclosure, the periodic control signal is a periodic square wave signal.

According to another aspect of the embodiment of the present disclosure, both the first AC signal and the second AC signal have a field strength of at least 1 V/cm.

According to another aspect of the embodiment of the present disclosure, the insulated electrode used for applying an electric field to a tumor of the patient's torso when treating tumor includes a plurality of electrode units arranged in an array, a plurality of connecting portions each connecting with two adjacent electrode units, and a wire connecting with the plurality of electrode units, the number of electrode units is at least 10 and the electrode units are arranged in at least three rows and four columns, and each electrode unit is connected with at least two electrode units adjacent thereto, and at least one adjacent two electrode units among the plurality of electrode units are arranged in spaced rows or spaced columns.

According to another aspect of the embodiment of the present disclosure, at least two adjacent electrode units among the plurality of electrode units are arranged in a disconnected manner and a gap is formed between the two adjacent electrode units arranged in a disconnected manner.

According to another aspect of the embodiment of the present disclosure, the insulated electrode further includes a wiring portion electrically connected to the connection portion or the electrode unit, and the wiring portion passes through the gap and is welded to the wire.

According to another aspect of the embodiment of the present disclosure, two adjacent electrode units arranged in rows are arranged in spaced columns, and among the plurality of electrode units arranged in columns, at least two adjacent electrode units in the same column are arranged in a spaced row.

According to another aspect of the embodiment of the present disclosure, the distances between a plurality of two adjacent electrode units arranged in rows are same, and the distances between a plurality of two adjacent electrode units arranged in columns are different.

According to another aspect of the embodiment of the present disclosure, the connecting portions located between a plurality of two adjacent electrode units arranged in the same row have the same length, and the connecting portions located between a plurality of two adjacent electrode units arranged in the same column have different lengths.

According to another aspect of the embodiment of the present disclosure, the insulated electrode has 13 electrode units distributed in an area defined by five rows and five columns.

According to another aspect of the embodiment of the present disclosure, at least two adjacent electrode units among the plurality of electrode units arranged in rows are arranged in spaced column, and the plurality of electrode units arranged in columns are all arranged in adjacent rows.

According to another aspect of the embodiment of the present disclosure, the distances between a plurality of two adjacent electrode units arranged in rows are different, and the distances between a plurality of two adjacent electrode units arranged in columns are same.

According to another aspect of the embodiment of the present disclosure, the connecting portions located between a plurality of two adjacent electrode units arranged in the same row have different lengths, and the connecting portions located between a plurality of two adjacent electrode units arranged in the same column have the same length.

According to another aspect of the embodiment of the present disclosure, the insulated electrode has 13 electrode units distributed in an area defined by three rows and five columns.

According to another aspect of the embodiment of the present disclosure, the connecting portion includes a first connecting portion connecting with two adjacent electrode units located in the same row and a second connecting portion connecting with two adjacent electrode units located in the same column.

According to another aspect of the embodiment of the present disclosure, the connecting portion further includes a third connecting portion connecting with two adjacent electrode units arranged diagonally in adjacent rows and adjacent columns.

According to another aspect of the embodiment of the present disclosure, the length of the third connecting portion is greater than that of the first connecting portion.

According to another aspect of the embodiment of the present disclosure, the length of the third connecting portion is greater than half of the length of the first connecting portion.

According to another aspect of the embodiment of the present disclosure, the length of the third connecting portion is greater than that of the second connecting portion.

According to another aspect of the embodiment of the present disclosure, the insulated electrode applying an alternating electric field to a tumor site of a patient's torso during treating tumor includes a plurality of electrode units distributed in an array area of at least three rows and four columns, a plurality of connecting portions each located between two adjacent electrode units and a wiring portion electrically connecting with all the electrode units, each electrode unit is connected to at least two adjacent electrode units through the connecting portion, and the electrode units are spaced with each other to form a plurality of open spaces located therebetween for allowing moisture on the patient's body surface to escape, the insulated electrode has at least 10 electrode units, and at least one adjacent two electrode units are arranged in a disconnected manner.

According to another aspect of the embodiment of the present disclosure, the plurality of electrode units are distributed in an interval manner in an area from 109 mm×109 mm to 219 mm×163 mm.

According to another aspect of the embodiment of the present disclosure, one open space has a largest area of 1065 mm², and one open space has a smallest area of 470 mm².

According to another aspect of the embodiment of the present disclosure, a distance of two adjacent electrode units arranged in the same row is about 23 mm to 50 mm.

According to another aspect of the embodiment of the present disclosure, the distance between two adjacent electrode units in the same row is 23 mm.

According to another aspect of the embodiment of the present disclosure, a length of the connecting portion connecting with two electrode units arranged in the same column and in adjacent rows is approximately 1 mm to 28 mm.

According to another aspect of the embodiment of the present disclosure, the length of the connecting portion connecting with two electrode units arranged in the same column and in adjacent rows is approximately 15 mm.

According to another aspect of the embodiment of the present disclosure, a width of the connecting portion is 4.5 mm to 6 mm.

According to another aspect of the embodiment of the present disclosure, the wiring portion is located between two adjacent electrode units arranged in the disconnected manner and has a width of 4 mm to 8 mm.

According to another aspect of the embodiment of the present disclosure, the wiring portion extends from an electrode unit located at a periphery of the array toward a space formed between the two electrode units arranged in the disconnected manner.

According to another aspect of the embodiment of the present disclosure, the insulated electrode further includes a reinforcing portion disposed opposite to the wiring portion, the reinforcing portion and the connecting portion are respectively located at opposite sides of the connecting portion which extends to form the wiring portion.

According to another aspect of the embodiment of the present disclosure, the insulated electrode arranged at a corresponding position of a tumor site in a patient's torso includes an electrical functional assembly for applying the alternating electric signal to the patient's tumor site, and the electrical functional assembly includes a plurality of electrode units arranged in at least three rows and four columns, a plurality of connecting portion each located between adjacent electrode units and electrically connecting with two adjacent electrode units, and a wiring portion extending from one connecting portion, the plurality of the connecting portions each connecting with two adjacent electrode units arranged in rows or in columns have different lengths.

According to another aspect of the embodiment of the present disclosure, the connecting portions include a first connecting portion which connects with two adjacent electrode units and extends laterally to form the wiring portion and a plurality of second connecting portions each only connecting with two adjacent electrode units arranged in the same row or in the same column, and the first connecting portion connects with two adjacent electrode units arranged in the same row but in spaced columns or in same column but in spaced rows.

According to another aspect of the embodiment of the present disclosure, a length of the second connecting portion connecting with two adjacent electrode units located in the same row and adjacent columns is between 1 mm and 3 mm.

According to another aspect of the embodiment of the present disclosure, a length of the first connecting portion is between 22 mm and 27 mm.

According to another aspect of the embodiment of the present disclosure, the insulated electrode has 14 electrode units arranged in three rows and five columns.

According to another aspect of the embodiment of the present disclosure, the distance between the two adjacent electrode units arranged in the same row and adjacent columns is between 1 mm and 3 mm.

According to another aspect of the embodiment of the present disclosure, a distance between two adjacent electrode units arranged in the same column and adjacent rows is between 1 mm and 3 mm.

According to another aspect of the embodiment of the present disclosure, the insulated electrode includes an electrical functional assembly for applying the alternating electric signal to a tumor of the patient's torso, and the electrical functional assembly includes at least 10 electrode units arranged in at least three rows and four columns, a plurality of connecting portion each connecting with two adjacent electrode units and a wiring portion connecting with the connecting portion, each electrode unit is connected with at least two connecting portions, numbers of the electrode units arranged in rows or columns are not exactly same.

According to another aspect of the embodiment of the present disclosure, the insulated electrode has 20 electrode units distributed in an array area surrounded by four rows and six columns.

According to another aspect of the embodiment of the present disclosure, at least two adjacent electrode units arranged in the same row or in the same column are arranged in a disconnected manner.

According to another aspect of the embodiment of the present disclosure, the insulated electrode includes an electrical functional assembly, and the electrical functional assembly has a space formed between the two adjacent electrode units arranged in a disconnected manner, and the wiring portion passes through the space.

According to another aspect of the embodiment of the present disclosure, the wiring portion extends from one connecting portion toward the space.

According to another aspect of the embodiment of the present disclosure, the wiring portion and the connecting portion are perpendicular to each other, and the wiring portion has a shape of “˜”.

According to another aspect of the embodiment of the present disclosure, the wiring portion is bridged between two connecting portions which respectively connects with two adjacent electrode units arranged in the disconnected manner.

According to another aspect of the embodiment of the present disclosure, the wiring portion is arranged substantially in a shape of “T”.

According to another aspect of the embodiment of the present disclosure, distances between a plurality of two adjacent electrode units arranged in rows are same, and the connecting portions each connecting with the two adjacent electrode units arranged in rows have a same length.

According to another aspect of the embodiment of the present disclosure, distances between a plurality of two adjacent electrode units arranged in columns are same, and the plurality of the connecting portions each connecting with the two adjacent electrode units arranged in columns have a same length.

According to another aspect of the embodiment of the present disclosure, at least one adjacent electrode units arranged in rows are arranged in spaced columns, and distances between a plurality of two adjacent electrode units arranged in rows are not exactly same.

According to another aspect of the embodiment of the present disclosure, at least one adjacent electrode units arranged in columns are arranged in spaced rows, and the distances between a plurality of two adjacent electrode units arranged in columns are not exactly same.

According to another aspect of the embodiment of the present disclosure, the adjacent electrode units arranged in rows are arranged in adjacent columns, and the distances between a plurality of two adjacent electrode units arranged in rows are same.

According to another aspect of the embodiment of the present disclosure, two adjacent electrode units arranged in columns are arranged in adjacent rows, and the distances between a plurality of two adjacent electrode units arranged in columns are same.

According to another aspect of the embodiment of the present disclosure, the distances between a plurality of two adjacent electrode units arranged in rows are same, and the distances between a plurality of two adjacent electrode units arranged in columns are same.

According to another aspect of the embodiment of the present disclosure, the plurality of electrode units are distributed in an array defined by four rows and six columns in such a way that first and last columns each has two electrode units and four middle columns each has four electrode units.

According to another aspect of the embodiment of the present disclosure, the plurality of the electrode units are arranged in an axisymmetric manner both along row and column directions.

According to another aspect of the embodiment of the present disclosure, the insulated electrode further includes a wire electrically connected to the electrical functional assembly, and the wire is welded to the wiring portion.

According to another aspect of the embodiment of the present disclosure, the insulated electrode further includes a backing supporting the electrical functional assembly, and the backing has a cable hole for the wire passing through.

According to another aspect of the embodiment of the present disclosure, the insulated electrode includes a plurality of electrode units arranged in at least three rows and four columns, a plurality of connecting portion each connecting with two adjacent electrode units, and a wiring portion connected to the connecting portion, and each electrode unit is connected with at least two connecting portions, the electrode units are arranged at intervals to form a plurality of open spaces located therebetween, and the wiring portion passes through one open space.

According to another aspect of the embodiment of the present disclosure, the distance between two adjacent electrode units arranged in the same row is 8 mm to 22 mm.

According to another aspect of the embodiment of the present disclosure, the distance between two adjacent electrode units arranged in the same column is 6.5 mm to 25 mm.

According to another aspect of the embodiment of the present disclosure, the distance between two adjacent electrode units diagonally arranged in adjacent rows and adjacent columns is 19 mm to 33.3 mm.

According to another aspect of the embodiment of the present disclosure, the plurality of electrode units are distributed at intervals in an area of 166 mm×103.5 mm to 242 mm×166 mm.

According to another aspect of the embodiment of the present disclosure, a width of the connecting portion is 4.5 mm to 6 mm, or preferably, the width of the connecting portion is 4.5 mm.

According to another aspect of the embodiment of the present disclosure, at least two adjacent electrode units arranged in the same row or in the same column are arranged in a disconnected manner, and a space is formed between the two adjacent electrode units arranged in the disconnected manner.

According to another aspect of the embodiment of the present disclosure, the wiring portion passes through the gap and includes a bridging section bridging between two connecting portions and a wiring section extending from the bridging section toward the gap.

According to another aspect of the embodiment of the present disclosure, a width of the bridging section of the wiring portion is 4.5 mm to 6 mm, and a width of the wiring section of the wiring portion is 4 mm to 8 mm.

According to another aspect of the embodiment of the present disclosure, the insulated electrode has 20 electrode units distributed in an array area surrounded by four rows and six columns, one open space has a largest area of 4428 mm², and one open space has a smallest area of 452 mm².

According to another aspect of the embodiment of the present disclosure, the insulated electrodes include an electrical functional assembly for applying an alternating electric signal to the patient's tumor site and a wire electrically connected to the electrical functional assembly, and the electrical functional assembly includes at least 10 electrode units, a plurality of connecting portion each connecting with two adjacent electrode units and a wiring portion electrically connected to a wire, each electrode unit is connected with at least two connecting portions.

According to another aspect of the embodiment of the present disclosure, each electrode unit is connected with at least two adjacent electrode units thereof.

According to another aspect of the embodiment of the present disclosure, the plurality of electrode units are distributed in an array area of at least three rows and four columns, and the number of the electrode units is at least 10 and at most 30.

According to another aspect of the embodiment of the present disclosure, the plurality of electrode units are distributed in an array area of at least three rows and four columns, and the electrode units arranged in each row have the same number thereof and are arranged in a column-wise alignment.

According to another aspect of the embodiment of the present disclosure, the electrode units are arranged with a same row spacing.

According to another aspect of the embodiment of the present disclosure, the electrode units are arranged with a same column spacing.

According to another aspect of the embodiment of the present disclosure, the plurality of the connecting portions each connecting with two adjacent electrode units arranged in rows have a same length.

According to another aspect of the embodiment of the present disclosure, the plurality of connecting portions each connecting with two adjacent electrode units arranged in columns have a same length.

According to another aspect of the embodiment of the present disclosure, at least two adjacent electrode units located in the same row or in the same column are arranged in a disconnected manner, and a gap is formed between the two adjacent electrode units arranged in the disconnected manner for the wiring portion passing therethrough.

According to another aspect of the embodiment of the present disclosure, the wiring portion extends laterally from the connecting portion which is opposite to the gap.

According to another aspect of the embodiment of the present disclosure, the wiring portion is perpendicular to the connecting portion which extends to form the wiring portion.

According to another aspect of the embodiment of the present disclosure, the wiring portion is bridged between two connecting portions respectively connected to the two electrode units arranged in the disconnected manner.

According to another aspect of the embodiment of the present disclosure, the wiring portion is arranged in a substantially “T” shape.

According to another aspect of the embodiment of the present disclosure, the insulated electrode has 20 electrode units arranged in an array of four rows and five columns.

According to another aspect of the embodiment of the present disclosure, the electrode unit includes a main body disposed at one end of the connecting portion, an insulating plate disposed on one side of the main body that is away from the human skin, and a dielectric element disposed on one side of the main body that faces toward the human skin.

According to another aspect of the embodiment of the present disclosure, the electrode unit further includes a temperature sensor selectively arranged on the main body, and the temperature sensor and the dielectric element are located on the same side of the main body.

According to another aspect of the embodiment of the present disclosure, the dielectric element has a through hole corresponding to the temperature sensor.

According to another aspect of the embodiment of the present disclosure, the insulated electrode further includes an adhesive backing supporting the electrical functional assembly, and the backing is provided with a cable hole for the wire passing through.

According to another aspect of the embodiment of the present disclosure, the wire is provided with a heat-shrinkable sleeve covering a joint formed between the wire and the wiring portion.

According to another aspect of the embodiment of the present disclosure, the insulated electrode includes a flexible circuit board, a dielectric element and a plurality of temperature sensors arranged on the same side of the flexible circuit board and a wire electrically connected to the flexible circuit board; the number of the temperature sensors is n, and n is an integer which is greater than 1 but equal to or less than 8; each temperature sensor has a ground terminal and a signal terminal; the flexible circuit board has an insulating substrate and a plurality of conductive traces embedded in the insulating substrate; the number of the conductive trances is n plus 2; one conductive trace is electrically connected to the dielectric element, one conductive trace is electrically connected with the ground terminals of all the temperature sensors, and the remaining conductive traces each is respectively connected to the signal terminal of corresponding temperature sensor; and the wire is electrically connected to all the conductive traces of the flexible circuit board.

According to another aspect of the embodiment of the present disclosure, the flexible circuit board has a plurality of gold fingers each exposed from the insulating substrate and electrically connected to corresponding portion of the wire.

According to another aspect of the embodiment of the present disclosure, the gold fingers each is connected to one corresponding conductive trace of the flexible circuit board.

According to another aspect of the embodiment of the present disclosure, the number of the temperature sensors is 2, the number of the conductive traces is 4, and the number of the gold fingers is 4.

According to another aspect of the embodiment of the present disclosure, the flexible circuit board is provided with a conductive pad corresponding to the dielectric element, and the conductive pad is welded to the dielectric element.

According to another aspect of the embodiment of the present disclosure, the conductive pad exposes from the insulating substrate and is connected to a conductive trace electrically connecting the flexible circuit board to the dielectric element.

According to another aspect of the embodiment of the present disclosure, the conductive pad includes a plurality of conductive cores arranged at intervals, and the plurality of conductive cores are connected in series by one conductive trace electrically connecting the flexible circuit board to the dielectric element.

According to another aspect of the embodiment of the present disclosure, the flexible circuit board is provided with n pairs of pads, and each pair of pads are located between two corresponding conductive cores arranged at intervals.

According to another aspect of the embodiment of the present disclosure, each pair of pads are provided at positions of the flexible circuit board corresponding to the corresponding temperature sensor and expose from the insulating substrate of the flexible circuit board.

According to another aspect of the embodiment of the present disclosure, each pair of pads include a first pad soldered with the ground terminal of the corresponding temperature sensor and a second pad welded to the signal terminal of the corresponding temperature sensor.

According to another aspect of the embodiment of the present disclosure, the first pad is connected to one conductive trace electrically connecting the flexible circuit board with the ground terminal of the temperature sensor, and the second pads each is connected to one corresponding conductive trace which electrically connects the flexible circuit board with the signal terminal of the corresponding temperature sensor.

According to another aspect of the embodiment of the present disclosure, the wire has one end electrically connected to the flexible circuit board and the other end provided with a plug.

According to another aspect of the embodiment of the present disclosure, a heat-shrinkable sleeve is provided at a joint between the wire and the flexible circuit board.

According to another aspect of the embodiment of the present disclosure, the dielectric element has through holes each corresponding to the temperature sensor, and each temperature sensor is accommodated in the corresponding through hole.

According to another aspect of the embodiment of the present disclosure, among the plurality of conductive traces, one conductive trace electrically connected to the dielectric element is a first conductive trace, one conductive trace electrically connected to the ground terminal of the temperature sensor is a second conductive trace, and the remaining n conductive traces respectively and electrically connected to the signal terminal of the corresponding temperature sensors are all third conductive traces, the flexible circuit board has a conductive pad connected to the first conductive trace and n pairs of pads, and each pair of pads has one pad connected to the second conductive trace and the other pad connected to the corresponding third conductive trace.

According to another aspect of the embodiment of the present disclosure, the conductive pad and the pads are disposed on the same side of the flexible circuit board.

According to another aspect of the embodiment of the present disclosure, the conductive pad and the pads both expose from the insulating substrate of the flexible circuit board.

According to another aspect of the embodiment of the present disclosure, the flexible circuit board further has a plurality of gold fingers welded to the wire and exposing from the insulating substrate thereof, and the number of gold fingers is n plus 2, and n is an integer which is greater than 1 but equal to or less than 8.

According to another aspect of the embodiment of the present disclosure, the number of the golden fingers is four, the number of the temperature sensors is two, the number of the pairs of the pads is two, and the number of the third conductive trace is two.

According to another aspect of the embodiment of the present disclosure, the gold finger, the conductive pad and the two pairs of pads are located at the same side of the flexible circuit board.

According to another aspect of the embodiment of the present disclosure, the insulated electrode further includes a backing adhered to a corresponding part of the flexible circuit board.

According to another aspect of the embodiment of the present disclosure, the insulated electrode further includes an insulating plate located at one side of the flexible circuit board that is away from the dielectric element, the insulating plate corresponds to the dielectric element along a thickness direction and is sandwiched between the flexible circuit board and the backing.

According to another aspect of the embodiment of the present disclosure, the insulated electrode includes at least one electrode plate capable of applying an alternating electric signal and an electrical connector detachably connected to the electrode plate, and the electrode plate includes an individual electrode unit and a first wire electrically connected to the electrode unit, and the electrode plate is detachably connected to the electrical connector through the first wire.

According to another aspect of the embodiment of the present disclosure, a plurality of the electrode plates are connected in parallel to the electrical connector through the corresponding first wire.

According to another aspect of the embodiment of the present disclosure, the first wire of the electrode plate has a first plug detachably plugged into the electrical connector, and the first plug and the electrode unit are respectively located at opposite ends of the first wire.

According to another aspect of the embodiment of the present disclosure, the electrical connector has a plurality of sockets into which the first plug of the first wire of the corresponding electrode plate can be detachably plugged.

According to another aspect of the embodiment of the present disclosure, the electrical connector is provided with a second wire, and the second wire and the plurality of sockets are respectively located at opposite ends of the electrical connector.

According to another aspect of the embodiment of the present disclosure, the second wire has a second plug disposed at an end thereof.

According to another aspect of the embodiment of the present disclosure, the electrical connector has a body, and the plurality of sockets and the second wire are respectively arranged at opposite ends of the body.

According to another aspect of the embodiment of the present disclosure, the electrode plate further includes a wiring portion connected to the electrode unit, and the wiring portion is welded to an end of the first wire that is away from the first plug.

According to another aspect of the embodiment of the present disclosure, the electrode unit includes a main body and a dielectric element welded on one side of the main body, and the wiring portion is laterally extended from the main body.

According to another aspect of the embodiment of the present disclosure, the main body portion of the electrode unit and the wiring portion form a flexible circuit board of the electrode plate.

According to another aspect of the embodiment of the present disclosure, the electrode unit further includes at least one temperature sensor, and the temperature sensor is disposed on the main body, and the temperature sensor and the dielectric element are located on the same side.

According to another aspect of the embodiment of the present disclosure, at least one through-hole is provided in the middle of the dielectric element, and the temperature sensors are respectively accommodated in corresponding through-holes of the dielectric element.

According to another aspect of the embodiment of the present disclosure, the electrode unit further includes an insulating plate adhered to a side of the main body that is away from the dielectric element.

According to another aspect of the embodiment of the present disclosure, a heat-shrinkable sleeve is wrapped around a welded joint formed between the first wire and the wiring portion.

According to another aspect of the embodiment of the present disclosure, the first wire is detachably connected to the electrode unit.

According to another aspect of the embodiment of the present disclosure, the electrode plate includes a wiring portion electrically connected to the electrode unit, and a docking socket is provided at an end of the wiring portion that is away from the electrode unit.

According to another aspect of the embodiment of the present disclosure, a docking plug is disposed at an end of the first wire that is away from the first plug, and the docking plug is detachably plugged into the docking socket.

According to another aspect of the embodiment of the present disclosure, the electrode plate further includes a backing adhered to the electrode unit, a support member which is arranged around the electrode unit and adhered to the backing and an adhesive member covering sides of the electrode unit and the support member that are away from the backing.

According to another aspect of the embodiment of the present disclosure, the insulated electrode includes a backing, a flexible circuit board disposed on the backing, a dielectric element and a heat dissipation reinforcing member; the heat dissipation reinforcing member and the dielectric element are respectively located on opposite sides of the flexible circuit board, the heat dissipation reinforcing member is sandwiched between the flexible circuit board and the backing, and the heat dissipation reinforcing member is made of a material with a thermal conductivity greater than 200 W/mK.

According to another aspect of the embodiment of the present disclosure, at least one heat dissipation hole is provided on the heat dissipation reinforcing member.

According to another aspect of the embodiment of the present disclosure, the heat dissipation reinforcing member is a metal plate or a metal alloy plate or a graphene composite plate.

According to another aspect of the embodiment of the present disclosure, the heat dissipation reinforcing member is a metal plate or a metal alloy plate with a thickness of 0.1 mm to 0.7 mm.

According to another aspect of the embodiment of the present disclosure, the heat dissipation reinforcing member is an aluminum plate or an aluminum alloy plate with a thickness of 0.3 mm to 0.6 mm.

According to another aspect of the embodiment of the present disclosure, the heat dissipation reinforcing member is an aluminum plate with a thickness of 0.6 mm.

According to another aspect of the embodiment of the present disclosure, the number of the heat dissipation holes is 30 and the heat dissipation holes are uniformly distributed on the aluminum plate, and a diameter of the heat dissipation hole is 0.5 mm.

According to another aspect of the embodiment of the present disclosure, the heat dissipation reinforcing member is an aluminum alloy plate with a thickness of 0.3 mm and a thermal conductivity of 201 W/mK.

According to another aspect of the embodiment of the present disclosure, the number of the heat dissipation holes is 50 and the heat dissipation holes are uniformly distributed on the aluminum alloy plate, and a diameter of the heat dissipation hole is 0.4 mm.

According to another aspect of the embodiment of the present disclosure, the heat dissipation reinforcing member is a graphene composite plate with a thickness of 0.1 mm and a thermal conductivity greater than 300 W/mK.

According to another aspect of the embodiment of the present disclosure, the insulated electrode includes a flexible backing and an electrical functional assembly supported by the flexible backing; and the electrical functional assembly includes a dielectric element closer to a body surface corresponding to a patient's target area and a heat dissipation reinforcing member corresponding to the dielectric element; and the heat dissipation reinforcing member is sandwiched between the dielectric element and the flexible backing and made of a material with a thermal conductivity greater than 200 W/mK.

According to another aspect of the embodiment of the present disclosure, the heat dissipation reinforcing member is disposed on a side of the dielectric element that is away from the body surface of the patient's target area, and at least one heat dissipation hole is formed through the heat dissipation reinforcing member.

According to another aspect of the embodiment of the present disclosure, the heat dissipation reinforcing member is a metal plate or a metal alloy plate with a thickness of 0.1 mm to 0.7 mm.

According to another aspect of the embodiment of the present disclosure, the heat dissipation reinforcing member is an aluminum plate or an aluminum alloy plate with a thickness of 0.3 mm to 0.6 mm.

According to another aspect of the embodiment of the present disclosure, the heat dissipation reinforcing member is an aluminum plate with a thickness of 0.6 mm, the number of the heat dissipation holes is 30 and the heat dissipation holes are evenly distributed on the aluminum plate, and a diameter of the heat dissipation hole is 0.5 mm.

According to another aspect of the embodiment of the present disclosure, the heat dissipation reinforcing member is an aluminum alloy plate with a thickness of 0.3 mm and a thermal conductivity of 201 W/mK, and the number of the heat dissipation holes is 50 and the heat dissipation holes are uniformly distributed on the aluminum alloy plate, and a diameter of the heat dissipation hole is 0.4 mm.

According to another aspect of the embodiment of the present disclosure, the heat dissipation reinforcing member is a graphene composite plate with a thickness of 0.1 mm and a thermal conductivity greater than 300 W/mK.

According to another aspect of the embodiment of the present disclosure, the insulated electrode further includes a support member that is attached to the flexible backing and placed around the dielectric element and an adhesive member covering the support member and the dielectric element; the electrical function assembly also includes a temperature sensor contacting with the adhesive member and being configured to detect temperature of the adhesive member; and the heat dissipation reinforcing member is electrically insulated with the dielectric element.

According to another aspect of the embodiment of the present disclosure, the insulated electrode includes an electrical functional assembly, and the electrical functional assembly includes a flexible circuit board, a dielectric element and a temperature sensor both arranged on a side of the flexible circuit board that is faces toward the patient's skin, a semiconductor refrigerator arranged on a side of the flexible circuit board that faces away from the dielectric element, and the semiconductor refrigerator is arranged corresponding to the dielectric element along a thickness direction of the flexible circuit board.

According to another aspect of the embodiment of the present disclosure, the semiconductor refrigerator includes a refrigeration end disposed closer to the flexible circuit board and a heat dissipation end disposed opposite to the refrigeration end, and the refrigeration end is electrically connected to the flexible circuit board.

According to another aspect of the embodiment of the present disclosure, the semiconductor refrigerator further includes an N-type semiconductor and a P-type semiconductor sandwiched abreast between the refrigeration end and the heat dissipation end.

According to another aspect of the embodiment of the present disclosure, the semiconductor refrigerator further includes a sealant filled between the refrigeration end and the heat dissipation end for sealing the N-type semiconductor and the P-type semiconductor.

According to another aspect of the embodiment of the present disclosure, the refrigerating end of the semiconductor refrigerator has welding pads welded to the flexible circuit board, and the welding pads includes a positive electrode welding pad and a negative electrode welding pad.

According to another aspect of the embodiment of the present disclosure, the refrigerating end of the semiconductor refrigerator includes a cold-end ceramic plate arranged on a side of the flexible circuit board that is away from the patient's skin, a cold-end thermal conduction element arranged on the cold-end ceramic plate and two cold-end metal conductors arranged on the cold-end thermal conduction member at intervals; and the two cold-end metal conductors are respectively connected to the N-type semiconductor and the P-type semiconductor.

According to another aspect of the embodiment of the present disclosure, the welding pad is provided on a side of the cold-end ceramic plate that faces toward the flexible circuit board, and the flexible circuit board has a welding portion corresponding to the welding pad of the cold-end ceramic plate, and the welding portion is connected to the welding pad to realize the electrical connection between the cold-end ceramic plate and the flexible circuit board.

According to another aspect of the embodiment of the present disclosure, the cold-end ceramic plate is further provided with conductive traces electrically connected to the positive electrode welding pad and the negative electrode welding pad, and the cold-end thermal conduction element is provided with conductive traces corresponding to the conductive traces of the cold-end ceramic plate; and the two cold-end metal conductors are respectively electrically connected to the positive electrode welding pad and the negative electrode welding pad of the cold-end ceramic plate through the corresponding conductive traces of the cold-end thermal conduction element and the cold-end ceramic plate.

According to another aspect of the embodiment of the present disclosure, the heat dissipation end of the semiconductor refrigerator includes a hot-end ceramic plate, a hot-end thermal conduction element disposed on a side of the hot-end ceramic plate which is closer to the refrigeration end, and a heat-end metal conductor disposed on the hot-end thermal conduction element and contacting with the N-type semiconductor and the P-type semiconductor.

According to another aspect of the embodiment of the present disclosure, the N-type semiconductor and the P-type semiconductor are sandwiched between the hot-end metal conductor and the two cold-end metal conductors.

According to another aspect of the embodiment of the present disclosure, the N-type semiconductor, the P-type semiconductor, the cold-end metal conductors and the hot-end metal conductor are all made of the same material.

According to another aspect of the embodiment of the present disclosure, the positive electrode welding pad of the cold-end ceramic plate is electrically connected to the N-type semiconductor, and the negative electrode welding pad of the cold-end ceramic plate is electrically connected to the P-type semiconductor.

According to another aspect of the embodiment of the present disclosure, the temperature sensor and the semiconductor refrigerator are respectively arranged on opposite sides of the flexible circuit board, the dielectric element has a through hole extending therethrough and the temperature sensor is accommodated in the through hole.

According to another aspect of the embodiment of the present disclosure, the insulated electrode further includes a backing, and the electrical functional assembly is attached to the backing through the hot-end ceramic plate of the semiconductor refrigerator, and the semiconductor refrigerator is sandwiched between the backing and the flexible circuit board.

According to another aspect of the embodiment of the present disclosure, the backing has openings each located at a position corresponding to the hot-end ceramic plate of the semiconductor refrigerator to expose the hot-end ceramic plate of the corresponding semiconductor refrigerator to the air.

According to another aspect of the embodiment of the present disclosure, the tumor electrical field therapy system further includes a controller electrically connected to the insulated electrode, and the controller controls turn-on or turn-off of the semiconductor refrigerator of the insulated electrode, or turn-off of the tumor electric field therapy system by monitoring temperature of the insulated electrode.

According to another aspect of the embodiment of the present disclosure, the tumor-treating fields system uses the following temperature control method, the temperature control method includes: S1: monitoring temperature of the insulated electrode in real time; S2: determining whether the monitored temperature exceeds a regulating temperature or not; S3: according to a determining result in the step S2, turning off the semiconductor refrigerator of the insulated electrode or continuing to determine whether the monitored temperature exceeds a safety threshold or not; S4: according to a determining result of whether the monitored temperature exceeding the safety threshold system or not in step S3, turning on the semiconductor refrigerator of the insulated electrode or turning off the tumor electric field therapy system.

According to another aspect of the embodiment of the present disclosure, the temperature of the insulated electrode is monitored in real time by a temperature sensor.

According to another aspect of the embodiment of the present disclosure, the safety threshold is greater than the regulating temperature.

According to another aspect of the embodiment of the present disclosure, the difference between the safety threshold and the regulating temperature is within 4° C.

According to another aspect of the embodiment of the present disclosure, the regulating temperature is 39°, and the safety threshold is 41°.

According to another aspect of the embodiment of the present disclosure, according to a determining result in step S2, turning off the semiconductor refrigerator of the insulated electrode or continuing to determine whether the monitored temperature exceeds a safety threshold or not includes the following steps:
when the monitored temperature is lower than the regulating temperature, controlling the semiconductor refrigerator of the insulated electrode to be in a turn-off sate; and
when the monitored temperature is higher than or equal to the regulating temperature, determining whether the monitored temperature exceeding the safety threshold or not.

According to another aspect of the embodiment of the present disclosure, the determining result in step S2 includes the monitored temperature being lower than the regulating temperature and the monitored temperature being higher than or equal to the regulating temperature.

According to another aspect of the embodiment of the present disclosure, the determining result of the monitored temperature whether exceeding the safety threshold or not in step S3 includes the monitored temperature being lower than the safety threshold and the monitored temperature being higher than or equal to the safety threshold.

According to another aspect of the embodiment of the present disclosure, according to a determining result of whether the monitored temperature exceeding the safety threshold or not in step S3, turning on the semiconductor refrigerator of the insulated electrode or turning off the tumor electric field therapy system includes the following steps: when the monitored temperature is lower than the safety threshold, controlling the semiconductor refrigerator of the insulated electrode to be in turn-on state and repeatedly and periodically performing step S1 to step S3; when the monitored temperature is higher than or equal to the safety threshold, controlling the tumor electric field therapy system to be in a turn-off state.

According to another aspect of the embodiment of the present disclosure, turning on the semiconductor refrigerator of the insulated electrode is realized by supplying direct current to the semiconductor refrigerator.

According to the present disclosure, the electric field therapy device of the tumor electric field therapy system for applying an alternating electric signal to the tumor in a patient's torso is configured to increase the AC voltage value of the alternating electric signal outputted from the AC voltage control unit to the insulated electrodes arranged in pairs from 0 to the specific voltage at a constant speed during each switching-on time period t3 and decrease the AC voltage of the alternating electric signal applied from the AC voltage control unit to the insulated electrodes in pairs from the specific voltage to 0 at a constant speed during each switching-off time period t4 through MCU control unit controlling the DC power unit, so as to avoid damage of electronic components in the electric field therapy device and a tingling sensation in the experimental animals or humans both caused by a sharp change of the AC voltage of the alternating electric signal when the alternating electric signal is switched between different pairs of the insulated electrodes.

It is to be understood that both the foregoing general description and the following detailed description are exemplary and explanatory only and are not restrictive of the application.

### BRIEF DESCRIPTION OF THE DRAWINGS

In order to more clearly illustrate the technical solutions in the embodiment of the present disclosure, the drawings that need to be used in the description of the embodiment will be briefly introduced below. Obviously, the drawings in the following description are only some embodiment of the present disclosure. For those skilled in the art, other drawings can also be obtained based on these drawings without any creative effort.

FIG. 1 is a system block diagram of the tumor electric field therapy system of the present disclosure.

FIG. 2 is a schematic diagram of control signals for turning on or off the first electric field and the second electric field in the tumor electric field therapy system.

FIG. 3 is a schematic diagram for an AC signal applied to an insulated electrode.

FIG. 4 is a graph showing the relationship between the cell growth rate and the duty cycle of the electric field.

FIG. 5 is a three-dimensional assembled view of the first embodiment of the insulated electrodes of the tumor electric field therapy system according to the present disclosure.

FIG. 6 is an exploded perspective view of the insulated electrode shown in FIG. 5.

FIG. 7 is an exploded perspective view of the electrical functional assembly of the insulated electrode shown in FIG. 6.

FIG. 8 is a plan view of a dielectric element of the electrical functional assembly shown in FIG. 7.

FIG. 9 is a plan view of the flexible circuit board of the insulated electrodes shown in FIG. 6.

FIG. 10 is a three-dimensional combined view of an alternative embodiment of the insulated electrodes in the first embodiment of the tumor electric field therapy system of the present disclosure.

FIG. 11 is an exploded perspective view of the electrical functional assembly of the insulated electrode shown in FIG. 10.

FIG. 12 is a three-dimensional assembled view of the second embodiment of the insulated electrodes of the tumor electric field therapy system of the present disclosure.

FIG. 13 is a perspective view of the flexible circuit board and wires of the insulated electrodes shown in FIG. 12.

FIG. 14 is a perspective view of insulating electrodes in the third embodiment of the tumor electric field therapy system of the present disclosure.

FIG. 15 is an exploded perspective view of the insulated electrode shown in FIG. 14.

FIG. 16 is an exploded perspective view of the electrical functional assembly of the insulated electrode shown in FIG. 15.

FIG. 17 is a three-dimensional combined view of the electrical functional assembly in FIG. 15.

FIG. 18 is a three-dimensional assembled view of the fourth embodiment of the insulated electrodes of the tumor electric field therapy system of the present disclosure.

FIG. 19 is a bottom plan view of the insulated electrode shown in FIG. 18.

FIG. 20 is an exploded perspective view of the insulated electrode shown in FIG. 18.

FIG. 21 is an exploded perspective view of electrical functional assembly and wires of the insulated electrode shown in FIG. 20.

FIG. 22 is a plan view of a fifth embodiment of the insulated electrodes of the tumor electric field therapy system of the present disclosure.

FIG. 23 is an exploded perspective view of the insulated electrode shown in FIG. 22.

FIG. 24 is a plan view of the electrical functional assembly shown in FIG. 23.

FIG. 25 is a perspective combined view of the sixth embodiment of the insulated electrodes of the tumor electric field therapy system of the present disclosure.

FIG. 26 is an exploded perspective view of the insulated electrode shown in FIG. 25.

FIG. 27 is an exploded perspective view of electrical functional assembly and wires of the insulated electrode shown in FIG. 26.

FIG. 28 is a schematic plan view of a flexible circuit board with insulated electrodes shown in FIG. 27.

FIG. 29 is a front wiring diagram of the flexible circuit board of the electrical functional assembly shown in FIG. 28.

FIG. 30 is a rear wiring diagram of the flexible circuit board of the electrical functional assembly shown in FIG. 28.

FIG. 31 is similar to FIG. 26 with a slightly different backing for the insulated electrodes.

FIG. 32 is an exploded perspective view of an alternative implementation of the sixth embodiment of the insulated electrode of FIG. 26.

FIG. 33 is an exploded perspective view of electrical functional assembly and wires of the insulated electrode shown in FIG. 32.

FIG. 34 is a schematic plan view of a flexible circuit board with insulated electrodes shown in FIG. 33.

FIG. 35 is a three-dimensional assembled view of the seventh embodiment of the insulated electrodes of the tumor electric field therapy system of the present disclosure.

FIG. 36 is an exploded view of the electrode plate and the electrical connector of the insulated electrode shown in FIG. 35.

FIG. 37 is an exploded perspective view of an electrode piece of the insulated electrode shown in FIG. 36.

FIG. 38 is a three-dimensional exploded view of the electrode unit and the first wire of the electrode plate shown in FIG. 37.

FIG. 39 is a plan view of the flexible circuit board of the electrode plate shown in FIG. 38.

FIG. 40 is an exploded view of a variant embodiment of the insulated electrodes in the seventh embodiment of the tumor electric field therapy system of the present disclosure.

FIG. 41 is an exploded perspective view of the electrode plate shown in FIG. 40.

FIG. 42 is an exploded perspective view of the eighth embodiment of the insulated electrodes of the tumor electric field therapy system of the present disclosure.

FIG. 43 is an exploded perspective view of the electrical functional assembly of the insulated electrode shown in FIG. 42.

FIG. 44 is a schematic perspective view of the heat dissipation reinforcing member of the insulated electrode shown in FIG. 43.

FIG. 45 is an exploded perspective view of the insulated electrode in FIG. 42.

FIG. 46 is a three-dimensional exploded view of a partial structure of a variant embodiment of an insulated electrode in the eighth embodiment of the tumor electric field therapy system of the present disclosure.

FIG. 47 is a perspective view of the flexible circuit board of the electrical functional assembly shown in FIG. 46.

FIG. 48 is a perspective view of the semiconductor refrigerator with insulated electrodes shown in FIG. 47.

FIG. 49 is a cross-sectional view of the semiconductor refrigerator and the flexible circuit board in FIG. 48.

FIG. 50 is a flow chart of steps for temperature control of the tumor electric field therapy system with the insulated electrodes shown in FIG. 46.

FIG. 51 is a schematic flowchart of the temperature control of the tumor electric field therapy system with the insulated electrodes shown in FIG. 46.

### DETAILED DESCRIPTION

The following will clearly and completely describe the technical solutions in the embodiment of the application with reference to the drawings in the embodiment of the application. Apparently, the described embodiment are only some of the embodiment of the application, not all of them. Based on the embodiment in this application, all other embodiment obtained by persons of ordinary skill in the art without making creative efforts belong to the scope of protection of this application.

Reference will now be made in detail to the exemplary embodiment, examples of which are illustrated in the accompanying drawings. When the following description refers to the accompanying drawings, the same numerals in different drawings refer to the same or similar elements unless otherwise indicated. The implementations described in the following exemplary embodiment do not represent all implementations consistent with this application. Rather, they are merely examples of apparatuses and methods consistent with aspects of the present disclosure as recited in the appended claims.

The terminology used in this application is for the purpose of describing particular embodiment only, and is not intended to limit the application. As used in this application and the appended claims, the singular forms “a”, “the” and “the” may refer to the singular form and also include the plural form unless the context clearly indicates otherwise. It should also be understood that the term “and/or” as used herein refers to and encompasses any and all possible combinations of one or more of the associated listed items. Unless otherwise indicated, words “connected” or “connected” and similar terms are not limited to physical or mechanical connections, and may include electrical connections, whether direct or indirect.

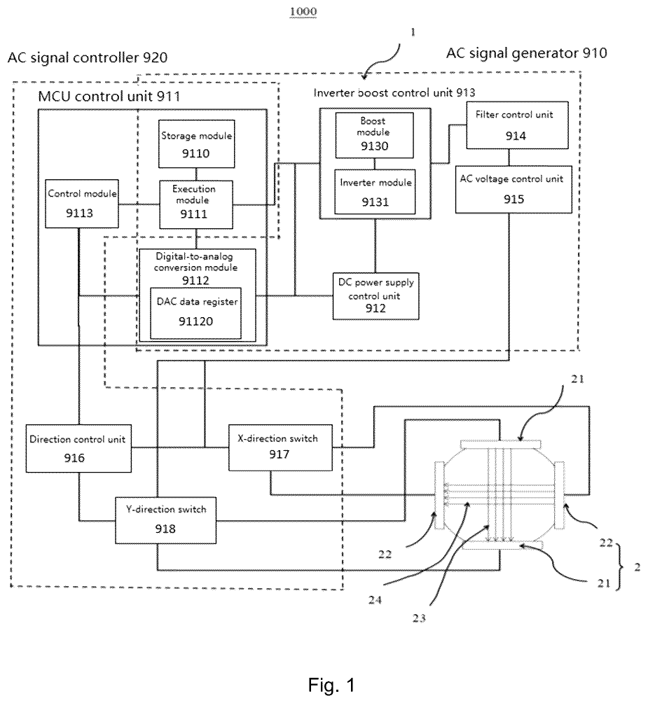

Referring to FIG. 1, a tumor electric field therapy system 1000 of the present invention is used to apply an alternating voltage to the tumor sites or the proliferating cells in tissue culture to treat tumors or inhibit proliferating cells in tissue culture, which includes an electric field therapy device 1 and two pairs of insulated electrodes 2 electrically connected to the electric field therapy device 1. The electric field therapy device 1 generates an alternating voltage signal for tumor treatment or inhibition of proliferating cells in tissue culture, and applies the generated alternating voltage signal cyclically and alternately between two pairs of insulated electrodes 2, thereby alternately generating an alternating electric field with changing directions between the two pairs of insulated electrodes 2. The electric field therapy device 1 includes an electric field generator (not shown) and an adapter (not shown) electrically connected to the electric field generator. The two pairs of insulated electrodes 2 include a pair of Y-direction electrodes 21 and a pair of X-direction electrodes 22 electrically connected to the electric field therapy device 1. The two Y-direction electrodes 21 are provided in parallel, and the two X-direction electrodes 22 are provided in parallel. The two Y-direction electrodes 21 and the two X-direction electrodes 22 are arranged perpendicular to each other. A Y-direction electric field 23 is generated between the two Y-direction electrodes 21. An X-direction electric field 24 is generated between the two X-direction electrodes 22. The X-direction electric field 24 is perpendicular to the Y-direction electric field 23.

The electric field therapy device 1 includes an MCU control unit 911, an inverter boost control unit 913 electrically connected to the MCU control unit 911, a DC power control unit 912 communicatively connected to the MCU control unit 911 and the inverter boost control unit 913, a filter control unit 914 electrically connected to the inverter boost control unit 913, an AC voltage control unit 915 electrically connected to the filter control unit 914, a direction control unit 916 electrically connected to the MCU control unit 911, an X-direction switch 917 electrically connected to the direction control unit 916 and controlling the on and off between the AC voltage control unit 915 and the two X-direction electrodes 22, and a Y-direction switch 918 electrically connected to the direction control unit 916 and controlling the on and off between the AC voltage control unit 915 and the two Y-direction electrodes 22.

The MCU control unit 911 has a reference voltage of 3.3V, which includes a storage module 9110, an execution module 9111 connected in communication with the storage module 9110, a digital-to-analog conversion module (DAC) 9112 connected in communication with the execution module 9111, and a control module 9113 controlling the storage module 9110, the execution module 9111 and the digital-to-analog conversion module 9112 to perform corresponding operations. The storage module 9110 is configured to store the system parameters of the electric field therapy device 1, which includes an electric field frequency, an output AC voltage amplitude, a direction switching period of the alternating electric signal, and the like.

The execution module 9111 is configured to read the electric field frequency, the output AC voltage amplitude, and the direction switching period of the alternating electrical signal stored in the electrical field therapy device 1. The execution module 9111 is also configured to output a periodic direction switching driving signal to the direction control unit 916 according to the direction switching period of the alternating electrical signal read from the electric field therapy device 1. The execution module 9111 is also configured to output a pulse signal to the inverter boost control unit 913 according to the electrical field frequency and the output AC voltage amplitude both read from the electric field therapy device 1 and the reference voltage of the MCU control unit 911. The pulse signal received by the inverter boost control unit 913 has a frequency same with the electrical field frequency read from the electrical field therapy device 1 and an AC voltage amplitude equal to the reference voltage amplitude of the MCU control unit 911. In this embodiment, the pulse signal outputted from the execution module 9111 to the inverter boost control unit 913 is a square wave with a frequency of 150 KHz, a voltage amplitude of 3.3V, and a duty cycle of 50%.

The digital-to-analog conversion module 9112 communicates with the DC power control unit 912, which has a DAC data register 91120 and can output a corresponding DC voltage to the DC power control unit 912 according to the digital value in the DAC data register 91120 to start the DC power control unit 912. The digital value corresponding to the DAC data register 91120 of the digital-to-analog conversion module 9112 and the reference voltage 3.3V of the MCU control unit 911 is 2¹². The control module 9113 controls the execution module 9111 to perform the mentioned corresponding functions, and the control module 9113 also controls on and off of the communication between the digital-to-analog conversion module 9112 and the DC power control unit 912 and whether the control execution module 9111 outputs a pulse signal to the inverter boost control unit 913 according to the direction switching period of the alternating electrical signal of the electric field therapy device 1 read by the execution module 9111.

The DC power control unit 912 receives a DC voltage signal of about 500 mv output from the digital-to-analog conversion module 9112 of the MCU control unit 911, and outputs a DC voltage signal of about 20V to the inverter boost control unit 913. The inverter boost control unit 913 has a boost module 9130 and an inverter module 9131 communicating with the boost module 9130. The boost module 9130 simultaneously receives a square wave with a frequency of 150 KHz, a voltage amplitude of 3.3V, and a duty cycle of 50% output from the execution module 9111 of the MCU control unit 911 and a 20V direct current signal output from the DC power control unit 912. The received square wave and the DC signal are superimposed and then boosted to output a square wave with a frequency of 150 KHz and an AC voltage amplitude of 80V to the inverter module 9131. The inverter module 9131 receives the square wave signal with a frequency of 150 KHz and a voltage amplitude of 80V output from the boost module 9130, and performs inversion processing on the received square wave signal to output the frequency to the filter control unit 914. It is a square wave of #80V. The filter control unit 914 performs filter processing on the received square wave with a frequency of 150 KHz and a voltage amplitude of +80V from the inverter module 9131 to obtain a sine wave with a frequency of 150 KHz and an AC voltage peak value of 160V, and sends the received signal to the AC voltage control unit 915 outputs a sine wave with a frequency of 150 KHz and a peak value of AC voltage of 160V after filtering. The AC voltage control unit 915 is connected to the X-direction switch 917 and the Y-direction switch 918 at the same time, and selectively converts the frequency processed by the filter control unit 914 to a sine wave of 150 KHz and an AC voltage peak value of 160V is applied to the two X-direction electrodes 22 or the two Y-direction electrodes 21 electrically connected to the AC voltage control unit 915, so as to generate an X-direction electric field 24 between the two X-direction electrodes 22 or generate a Y-direction electric field 23 between the two Y-direction electrodes 21 to treat tumors in experimental animals or to inhibit proliferation of cells in tissue culture.

The direction control unit 916 cyclically controls turn-on and turn-off of the X-direction switch 917 and the Y-direction switch 918 according to the periodic direction driving signal output by the execution module 9111 of the MCU control unit 911. Specifically, the control module 9113 of the MCU control unit 911 controls the execution module 9111 to output a periodic direction driving signal to the direction control unit 916 according to the direction switching period of the electric field therapy device 1 read by the execution module 9111, so as to alternately and circularly turn on the X-direction switch 917 and turn off the Y-direction switch 918 or turn off the X-direction switch 917 and turn on the Y-direction switch 918 through the direction control unit 916, therefore the sine wave with a frequency of 150 KHz and a peak AC voltage of 160V received by the AC voltage control unit 915 is periodically and alternately applied to two X-direction electrodes 22 or two Y-direction electrodes 21 both electrically connected to the AC voltage control unit 915 for generating a X-direction electric field 24 and a Y-direction electric field 23 cyclically and alternately applied to treat the malignant tumors in the experimental animals or inhibit the proliferating cells in the tissue culture.

That is, when the MCU control unit 911 controls the direction control unit 916 to turn on the X-direction switch 917 and turn off the Y-direction switch 918, the AC voltage control unit 915 applies a sine wave signal with a frequency of 150 KHz and AC voltage peak value of 160V, and generate X-direction electric field 24 between two X-direction electrodes 22. When the MCU control unit 911 controls the direction control unit 916 to turn off the X direction switch 917 and turn on the Y direction switch 918, the AC voltage control unit 915 applies a sine wave signal with a frequency of 150 KHz and an AC voltage peak value of 160V to the two Y-direction electrodes 21 electrically connected thereto, and generates a Y-direction electric field 23 between the two Y-direction electrodes 21. In this embodiment, the duty cycle of the periodic direction switching driving signal output from the execution module 9111 of the MCU control unit 911 to the direction control unit 916 is 50%, and the period is 2 S. That is, the direction control unit 916 controls the X-direction switch 917 to conduct in the first second, the Y-direction switch 918 to conduct in the second second, the X-direction switch 917 to conduct in the third second, and the Y-direction to conduct in the fourth second, and so on. The tumor electric field therapy system 1000 alternately applies an alternating voltage to the X-direction electrode 22 and the Y-direction electrode 21 through the cyclic conduction of the X-direction switch 917 and the Y-direction switch 918, so as to treat tumor sites in experimental animals or to inhibit proliferating cells in tissue cultures.

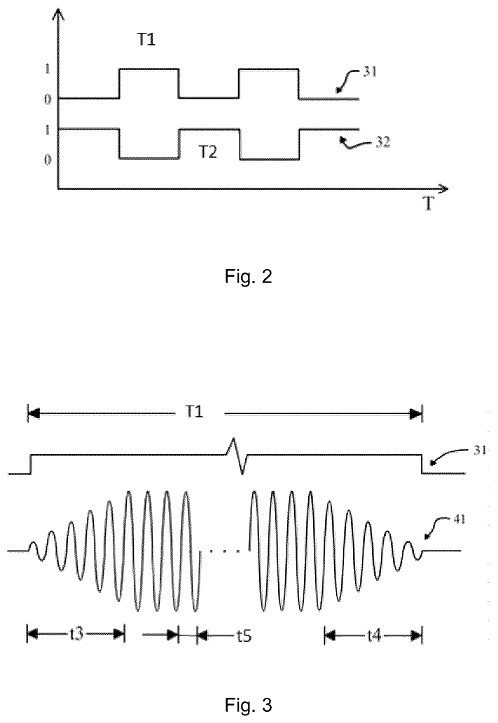

FIG. 2 is a driving signal waveform diagram for controlling the periodic direction switching between the electric field applied between the Y-direction electrode 21 and the X-direction electrode 22, that is the waveform diagram of the driving signal that the direction control unit 916 controls the X-direction switch 917 and the Y-direction switch 918. The driving signals 31 and 32 respectively correspond to the electrodes 22 in the X-direction and the electrodes 21 in the Y-direction. The duty ratios of the driving signals 31 and 32 are both 50%, and the periods are both 2 seconds. They have two output state with high level 1 and low level 0. The X-direction switch 917 and the Y-direction switch 918 are alternately turned on and off, and each switch is turned on and off for 1 second, and at the same time, there is only one of the X-direction switch 917 and the Y-direction switch 918 which is turned on. That is, when the X-direction switch 917 is turned on, the X-direction electrode 22 generates an X-direction AC electric field, and after the X-direction switch 917 is turned on for 1 second, the X-direction switch 917 is turned off, and the Y-direction switch 918 is turned on. The Y-direction electrode 21 generates a Y-direction AC electric field. The Y-direction switch 918 is turned on for one second, the Y-direction switch 918 is turned off, and the X-direction switch 917 is turned on again. The cycle repeats. The direction control unit 916 switches the X-direction switch 917 and the Y-direction switch 918 so that the target area is alternately subjected to the AC electric field in the Y-direction and the X-direction. The duty cycle and duty cycle of the driving signal can be adjusted and set arbitrarily according to the situation.

As shown in FIG. 2, during the T1 period, the X-direction switch 917 is turned on, and the AC voltage control unit 915 applies the AC signal to the two X-direction electrodes 22 (also called the first pair of insulated electrodes). An X-direction electric field 24 (also referred to the first electric field) with an intensity of at least 1 V/cm is generated, and at the same time, the Y-direction switch 918 is turned off, and the Y-direction electric field 23 is turned off. At this time, the driving signal 31 is at high level 1, and the driving signal 32 is at low level 0. During the T2 period, the AC voltage control unit 915 applies the AC signal to the two Y-direction electrodes 21 (also referred to as the second pair of insulated electrodes), and a Y-direction electric field 23 with a strength of at least 1 V/cm is generated between the two Y-direction electrodes 21 (also called the second electric field), and at the same time, the X-direction switch 917 is turned off, and the X-direction electric field 24 is turned off. At this time, the driving signal 31 is at low level 0, and the driving signal 32 is at high level 1. That is to say, the X-direction electric field 24 is switched to the Y-direction electric field 23 after working for a T1 period, and the Y-direction electric field 23 is switched to the X-direction electric field 24 after working for a T2 period. After repeating this, the driving signal 31, 32 realizes cyclic switching between the X-direction electric field 24 generated between the two X-direction electrodes 22 and the Y-direction electric field 23 generated between the two Y-direction electrodes 21. The T1 period is the working time of the electric field 24 in the X-direction in each cycle, and it is also the closing time of the electric field 23 in the Y-direction in each cycle. The T2 period is the working time of the electric field 23 in the Y-direction in each cycle. The off time of the electric field 24 in each cycle, in this embodiment, T1 and T2 are the same, which is half a cycle of the periodic switching driving signal generated by the AC signal controller 920. Both T1 and T2 are 1 second. Optionally, both T1 and T2 may also be between 400 ms and 1000 ms.

The storage module 9110, the execution module 9111, the digital-to-analog conversion module 9112, the control module 9113, the DC power control unit 912, the inverter boost control unit 913, the filter control unit 914 and the AC voltage control unit 915 of the MCU control unit 911 jointly form an AC signal generator 910 of the electric field therapy device 1. The storage module 9110, the execution module 9111, the control module 9113, the direction control unit 916 of the MCU control unit 911, and the X-direction switch 917 and the Y-direction switch 918 electrically connected to the direction control unit 916 jointly form an AC signal controller 920. When the AC voltage signal generated by the AC signal generator 910 is applied to the two X-direction electrodes 22, an X-direction electric field 24 is generated between the two X-direction electrodes 22. When the AC voltage signal generated by the AC signal generator 910 is applied to the two Y-direction electrodes 21, a Y-direction electric field 23 is generated between the two Y-direction electrodes 21.

Before the X-direction electric field 24 between the two X-direction electrodes 22 and the Y-direction electric field 23 between the two Y-direction electrodes 21 need to switch directions, the MCU control unit 911 disconnects the digital-to-analog conversion module 9112 from the DC power supply through the control module 9113, and the execution module 9111 is controlled by the control module 9113 to stop outputting pulse signals to the inverter boost control unit 913, so as to prevent the X-direction electric field 24 generated by the two X-direction electrodes 22 from being generated by the two Y-direction electrodes 21. The Y-direction electric field 23 is turned on at the same time to affect the treatment or inhibition effect. After the execution module 9111 stops outputting pulse signals to the inverter boost control unit 913, and the communication between the digital-to-analog conversion module 9112 and the DC power control unit 912 is disconnected, then the direction control unit 916 is controlled to switch the X-direction switch 917 and the Y-direction switch 918.

After the direction control unit 916 of the MCU control unit 911 finishes switching between the X-direction electric field 24 generated between the two X-direction electrodes 22 and the Y-direction electric field 23 generated between the two Y-direction electrodes 21, the control module 9113 of the MCU control unit 911 needs to control the digital-to-analog conversion module 9112 to output a voltage of 484 mV to the DC power control unit 912 to start the DC power control unit 912 so that the DC power control unit 912 outputs a 20V direct current signal to the inverter boost control unit 913, and simultaneously the control module 9113 controls the execution module 9111 to output a 150 KHz square wave signal to the inverter boost control unit 913, and then it is processed by the filter control unit 914 so that the AC voltage control unit 915 can output a sine wave of 150 KHz and an AC voltage peak value of 160V to two X-direction electrode 22 or two Y-direction electrodes 21. The value in the DAC data register 91120 corresponding to the 484 mV DC signal output by the digital-to-analog conversion module 9112 is 600 (484*4096/3300≈600).

The electric field therapy device 1 of the present invention controls the DC power control unit 912 to output the direct current signal in a way that the voltage change per unit time rises or falls constantly through the control module 9113 of the MCU control unit 911 according to the direction switching period of the alternating electrical signal acquired by the execution module 9111, and then the AC voltage applied between the two pairs of insulated electrodes 2 slowly rises or falls slowly in their respective work cycles, so as to avoid sudden changes in the AC voltage output by the AC voltage control unit 915 when the control direction control unit 916 completes the switching between the X-direction electric field 24 and the Y-direction electric field 23, causing spike signal impact and damaging the X-direction switch 917, the Y-direction switch 918 in the AC signal controller 920 or the AC voltage mutation generates spike pulses and transmits them to the X-direction electrodes 22 and Y-direction electrodes 21 arranged around the tumor site to cause a tingling sensation in experimental animals or humans. Please refer to the following description for details.

FIG. 3 is a schematic diagram of the periodic direction switching driving signal output by the MCU control unit 911 to the direction control unit 916 to generate an X-direction electric field 24 between two X-direction electrodes 22 for tumor electric field therapy, wherein the driving signal 31 is a partial waveform diagram of a periodic direction switching driving signal. The signal 41 is a sine wave applied to the two X-direction electrodes 22. The working time T1 of the electric field 24 in the X-direction is the continuous conduction time of the electric field in each cycle in this direction. The stage corresponding to the switching-on time period t3 of the alternating electrical signal is that the AC voltage applied to the two X-direction electrodes 22 is boosted from 0 to a specific value V (the value of the AC voltage applied to the insulating electrode 2 reaches 90% of the peak-to-peak value of the target voltage) process, the phase corresponding to the switching-off time period t4 of the alternating electrical signal is the process in which the AC voltage applied to the two X-direction electrodes 22 drops from a specific value V to 0. The specific value V is 90% of the peak value of the output AC voltage amplitude set by the electric field therapy device 1. Between the switching-on time period t3 and the switching-off time period t4, the alternating electrical signal also has several conduction periods t5 in which the stable output AC voltage is between a specific value V and the set output AC voltage peak value of the electric field therapy device 1.

As shown in FIG. 3, when T1 ends and switches to T2, that is, when the X-direction electric field 24 is turned off and the Y-direction electric field 23 is turned on, the AC voltage of the alternating electrical signal applied to the two X-direction electrodes 22 has dropped to 0V, which can effectively avoid the problem that the AC signal generator 910 applies voltage to the X-direction electrode 22 and the Y-direction electrode 21 at the same time because the AC voltage of the alternating electrical signal is switched before it drops to 0V when the AC signal on the X-direction electrode 22 is cut off, that is to avoid the situation that the X-direction electric field 24 and the Y-direction electric field 23 exist simultaneously and overlap. t3 and t4 are usually no more than 10% of T1, so that the time for the alternating electrical signal applied to the X-direction electrode 22 or the Y-direction electrode 21 to generate the electric field intensity that can achieve the therapeutic effect is as long as possible, and avoid reducing the electric field strength per unit time. The sum of the switching-on time period t3, the switching-off time period t4 and several conduction periods 15 is equal to T1. During the T2 period, the X-direction electric field 24 generated between the X-direction electrodes 22 is turned off, and the Y-direction electric field 23 generated between the Y-direction electrodes 21 is turned on, thereby completing a cycle switching. During the T2 period, the sine wave applied to the two Y-direction electrodes 21 is the same as the signal 41.

In order to eliminate the peak pulse, the MCU control unit controls the change of the value in the DAC data register to make the DC power control unit output a DC signal in a way that the voltage change within a unit time t is constant, thereby causing the AC voltage output to the AC voltage control unit is slowly boosted during the boost process or slowly decreased during the voltage reduction process. Divide the overall voltage boost V into n equal parts, and the AC voltage boost range within a unit time t is ΔV=V/n, which is defined that the AC voltage needs to be boosted from 0 to a specific voltage V within n unit time t, then the voltage change per unit time t is ΔV=V/n. The switching-on time period t3 of the alternating electrical signal is n*t, that is, n=t3/t, the voltage change ΔV within the unit time t is calculated by the following formula ΔV=V*t/t3 is obtained. Similarly, the voltage reduction process also adopts a constant voltage change ΔV voltage reduction to eliminate spikes, divide the overall voltage reduction amount into each unit time t, and define that the AC voltage needs to change from a specific voltage within n unit times t V is stepped down to 0, then the voltage change per unit time t is ΔV=V/n, and the alternating electrical signal switching-off time period t4 is n*t, that is, n=t4/t, the voltage change ΔV per unit time t is obtained by the following calculation formula ΔV=V*t/t4. That is to say, the AC voltage is boosted to a specific voltage V or dropped to 0 with a constant voltage change ΔV within a unit time t, and the boosting time and the voltage dropping time vary depending on the value of the specific voltage.

With reference to FIG. 3, the boost control and voltage reduction of different specific voltages V will be described in detail below.

In the first boosting implementation, the specific voltage is 100V, the unit time t is set to 1 ms, and the voltage change ΔV is 4V, then n=100/4=25, 13 is 25 ms. That is, the AC voltage applied by the AC voltage control unit 15 to the insulated electrode 2 is increased from 0 to 100V at a boost rate of 4V per millisecond for 25 ms. The value of the DAC data register corresponding to the AC voltage of 100V is 375, the value variable of the DAC data register per unit time t ΔDAC=375/25=15, and the MCU control unit increases the value of the DAC data register by 15 every millisecond. The output corresponding to the DAC data register value 4096 is 3.3V, and the output corresponding to the calculated DAC data register value 15 is (15*3.3/4096)*1000≈12 mV. That is to say, the MCU control unit increases the DAC output by about 12 mV per millisecond, so that the AC voltage control unit 15 boosts the voltage by 4V per millisecond, and lasts for 25 ms. After 25 uniform changes, the AC voltage applied to the insulating electrode 2 by the AC voltage control unit 15 changes from 0V t to 100V. When the voltage is boosted to 100V, even if the sudden change of the AC voltage applied by the AC voltage control unit 15 to the insulating electrode 2 has a 100% spike pulse, there is only a sudden change voltage of 4V, and the proportion of 4V to 100V is only 4%, because the sudden AC change voltage is still in the 5% error range of the voltage output, which can be ignored.

In the second boosting implementation, the specific voltage is 120V, the unit time t is set to 1 ms, and the voltage change ΔV is 4V, then n=120/4=30, 13 is 30 ms. That is, the MCU control unit controls the DAC output to increase by approximately 12 mV per millisecond so that the AC voltage control unit 15 boosts 4V per millisecond for 25 ms. After 25 uniform changes, the AC voltage applied to the insulated electrode 2 by the AC voltage control unit 15 is boosted from 0V to 100V. The value of the DAC data register corresponding to the AC voltage of 120V is 450, the value variable of the DAC data register per unit time t ΔDAC=450/30=15, and the MCU control unit increases the value of the DAC data register to 15 every millisecond. The output corresponding to the DAC data register value 4096 is 3.3V, and the output corresponding to the calculated DAC data register value 15 is (15*3.3/4096)*1000≈12 mV. That is to say, the MCU control unit controls the DAC data register to increase the output by about 12 mV every millisecond so that the AC voltage control unit 15 boosts the voltage by 4V every millisecond for 30 ms. After 30 uniform changes, the AC voltage of the AC voltage control unit 15 applied to the insulated electrode 2 is boosted from 0V to 120V. When boosting to 120V, even if there are 100% spikes in the voltage mutation, there is only a 4V mutation voltage, and the 4V ratio in 120V is only 3.3%, which still does not reach the 5% error range of the voltage output, which can be ignored.

In the third boosting implementation, the specific voltage is 160V, the unit time tis set to 1 ms, and the voltage change ΔV is 4V, then n=160/4=40, 13 is 40 ms, that is, boosting 4V per millisecond for 40 ms, the AC voltage applied by the AC voltage control unit 15 to the insulated electrode 2 increases from 0 to 160V. The value of the DAC data register corresponding to the AC voltage of 160V is 600, the value variable of the DAC data register per unit time t is ΔDAC=600/40=15, and the MCU control unit increases the value of the DAC data register to 15 every millisecond. The output corresponding to the DAC data register value 4096 is 3.3V, and the output corresponding to the calculated DAC data register value 15 is (15*3.3/4096)*1000≈12 mV. That is to say, the MCU control unit increases the output of the DAC data register by about 12 mV per millisecond, so that the AC voltage control unit 15 boosts the voltage by 4V per millisecond, lasts for 40 ms, and after uniformly changing 40 times, the AC voltage control unit 15 applies the AC voltage on the insulating electrode 2 Boost from 0V to 160V. When boosting to 160V, even if the voltage mutation has 100% spikes, there is only a 3.2V mutation voltage, and 4V accounts for only 2.5% at 160V, which has not yet reached the 5% error range of the voltage output, and the mutation voltage can be ignored.

In the above-mentioned boosting implementation, because the average voltage amplitude during t3 is too low to reach the target voltage value, the generated AC electric field strength is relatively low, which has little effect on the mitosis of cancer cells, and the working time T1 of the electric field in each cycle is 1 second.

The boosting process is a constant voltage boosting. Regardless of the specific value of the AC voltage, the amount of boosting per unit time t is the same. In the above embodiment, 4V is taken as an example, and the time of t3 will vary with the specific value of the AC voltage. The value becomes larger and longer, the longest time is 40 ms corresponding to 160V, the AC electric field duty cycle is 1 S, and the boost process accounts for 4% of the duty cycle, which has little effect on the overall treatment time.

In the first voltage reduction implementation method, the specific voltage is 100V, the unit time t is set to 1 ms, and the voltage change ΔV is 4V, then n=100/4=25, 14 is 25 ms, that is, the voltage reduction is 4V per millisecond for 25 ms, the AC voltage applied by the AC voltage control unit 15 to the insulating electrode 2 is reduced from 100V to 0. The DAC data register value corresponding to AC voltage 100V is 375, the DAC data register value variable ΔDAC=375/25=15 per unit time t, and the MCU control unit reduces the DAC data register value by 15 every millisecond. The output corresponding to the DAC data register value 4096 is 3.3V, and the output corresponding to the calculated DAC data register value 15 is (15*3.3/4096)*1000≈12 mV. That is to say, the MCU control unit lowers the DAC output by about 12 mV every millisecond to make the AC voltage control unit 15 reduce 4V every millisecond for 25 ms. After 25 uniform changes, the AC voltage applied to the insulating electrode 2 by the AC voltage control unit 15 is reduced from 100V to 0V. When reduced to 0V, even if there are 100% spikes in the voltage mutation, there is only a 4V mutation voltage, and 4V accounts for only 4% of 100V, which has not yet reached the 5% error range of the voltage output, and the mutation voltage can be ignored.

In the second voltage reduction implementation method, the specific voltage is 120V, the unit time t is set to 1 ms, and the voltage change ΔV is 4V, then n=120/4=30, t4 is 30 ms, that is, the voltage reduction is 4V per millisecond for 30 ms, the AC voltage applied by the AC voltage control unit 15 to the insulated electrode 2 is reduced from 120V to 0. The DAC data register value corresponding to the AC voltage 120V is 450, the DAC data register value variable ΔDAC=450/30=15 per unit time t, and the MCU control unit reduces the DAC data register value by 15 every millisecond. The output corresponding to the DAC data register value 4096 is 3.3V, and the output corresponding to the calculated DAC data register value 15 is (15*3.3/4096)*1000≈12 mV. That is to say, the MCU control unit lowers the DAC output by about 12 mV every millisecond to control the AC voltage control unit 15 to step down 4V every millisecond for 30 ms. The voltage is stepped down from 120V to 0V. When stepping down to 0V, even if the voltage mutation has 100% spikes, there is only a 4V mutation voltage, and 4V accounts for only 3.3% at 120V, which has not yet reached the 5% error range of the voltage output, and the mutation voltage can be ignored.

In the third voltage reduction implementation method, the specific voltage is 160V, the unit time t is set to 1 ms, and the voltage change ΔVis 4V, then n=160/4=40, 14 is 40 ms, that is, the voltage reduction is 4V per millisecond for 40 ms, and the AC voltage applied by the AC voltage control unit 15 to the insulating electrode 2 is dropped from 160V to 0. The DAC data register value corresponding to the AC voltage 160V is 600, the DAC data register value variable ΔDAC=600/40=15 per unit time t, and the MCU control unit reduces the DAC data register value by 15 every millisecond. The output corresponding to the DAC data register value 4096 is 3.3V, and the output corresponding to the calculated DAC data register value 15 is (15*3.3/4096)*1000≈12 mV. That is to say, the MCU control unit lowers the DAC output by about 12 mV per millisecond to control the AC voltage control unit 15 to step down 4V per millisecond for 40 ms. The voltage is stepped down from 160V to 0V. When stepping down to 0V, even if the voltage mutation has 100% spikes, there is only a 4V mutation voltage, and 4V accounts for only 2.5% at 160V, which has not yet reached the 5% error range of the voltage output, and the mutation voltage can be ignored.

In the above voltage reduction implementation method, because the average voltage amplitude during t4 is too low to reach the target voltage value, the generated AC electric field strength is relatively low, which has little effect on the mitosis of cancer cells, and the working time T1 of the electric field in each cycle is 1 second.

The voltage reduction process is a constant voltage reduction, regardless of the specific value of the AC voltage, the amount of voltage change ΔV per unit time tis constant. In the above embodiment, 4V is taken as an example, and the time of t4 will vary with the AC voltage. The specific voltage value becomes larger and longer, the longest time is 40 ms corresponding to 160V, the AC electric field duty cycle is 1 S, and the voltage reduction process accounts for 4% of the duty cycle, which has little effect on the overall treatment time. In this embodiment, the voltage change ΔV per unit time t in the step-up process and the voltage reduction process are the same, which is 4V.

The control module 9113 of the MCU control unit of the electric field therapy device of the tumor electric field therapy system 1000 of the present disclosure controls the constant voltage of the direct current signal output by the DC power control unit during the step-up process by controlling the numerical value change of the DAC data register per unit time. Boosting and stepping down the voltage at a constant voltage and slowly stepping down, so that the alternating voltage applied to the first pair of insulated electrodes 1 or the first pair of insulated electrodes 2 by the AC voltage control unit will automatically and slowly increase the voltage during the connection process and slowly reduce the voltage during the disconnection process, and control the voltage value changing per unit time within 5% of its working AC voltage, which can prevent the AC voltage applied to the first pair of insulated electrodes 1 and the first pair of insulated electrodes 2 from causing sudden changes during direction switching and damaging the X-direction switch 917 or Y-direction switch 918 of the AC signal controller 920, and also avoid the first pair of insulated electrodes 1 and the first pair of insulated electrodes 2 arranged on the body surface of the tumor site cause a tingling sensation in experimental animals or human bodies due to AC voltage mutations.

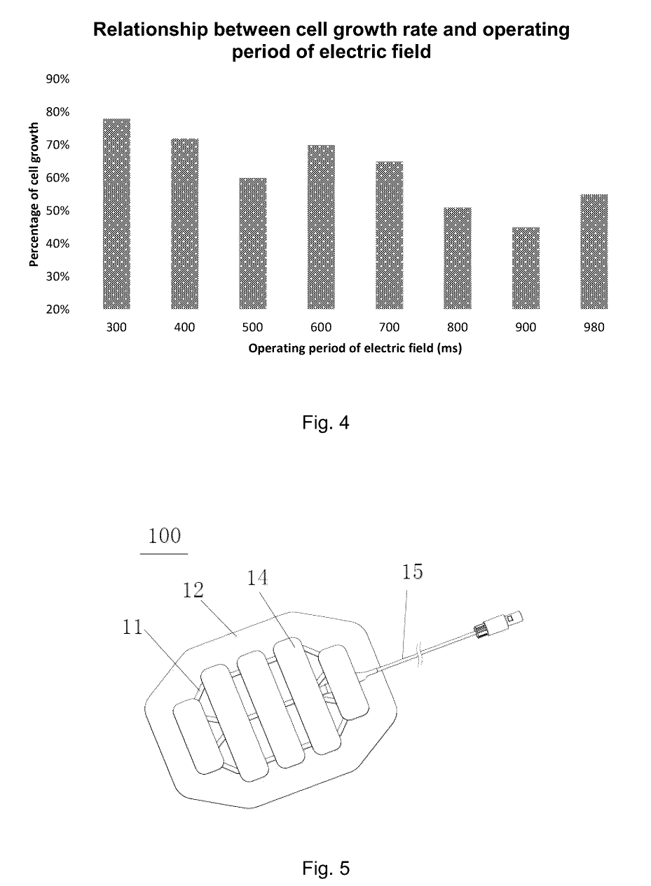

FIG. 4 shows the effect of electric fields with different working cycles on cell proliferation during the culture of glioma cells. The switching rates of the applied electric fields in different directions are different. The effect of the tumor treatment electric field on the proliferating cells in tissue culture and the inhibitory effects of malignant cells in experimental animals are different.

During experiments, it was found that when glioma cells were cultured in a culture dish and two pairs of mutually perpendicular 150 KHz AC signals were applied around them, changing the switching rate of the electric field 24 in the X-direction and the electric field 23 in the Y-direction would affect the proliferation of the cells. As shown in FIG. 2, the X-direction electric field 24 switches to the Y-direction electric field 23 after working for T1, and the Y-direction electric field 23 switches to the X-direction electric field 24 after working for T2, and so on, T1 and T2 are the same. The experimental results show that T1 and T2 have better inhibitory effect on cell proliferation at 400 ms to 980 ms than other rates. Preferably, when T1 and T2 are around 500 ms and between 700 ms and 980 ms, the inhibitory effect on cell proliferation is better. In this example, U87MG glioma was used as cell tissue culture, but the effect of its turnover rate on inhibiting cell proliferation is not limited to this cell, and other rapidly proliferating cells can also be applied.

The two Y-direction electrodes 21 and the two X-direction electrodes 22 in the tumor electric field therapy system 1000 have the same structure. The insulated electrodes of the present disclosure can have different embodiments. The insulated electrode of the present disclosure provides the following multiple embodiments:

### First Embodiment of the Insulated Electrodes

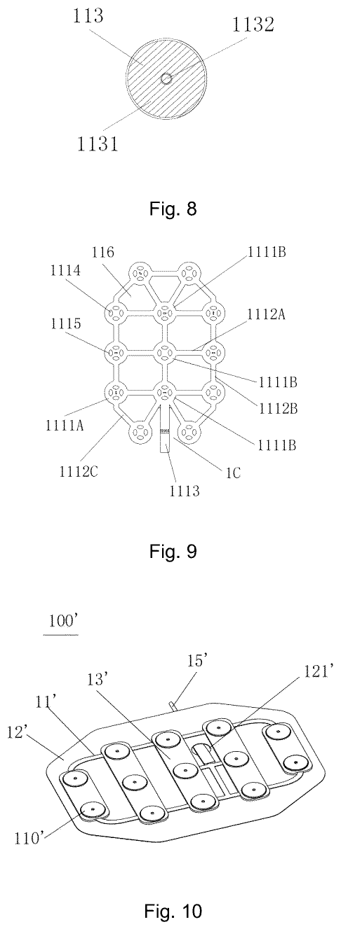

FIG. 5 to FIG. 9 show the insulated electrode 100 of the first embodiment of the present disclosure. The insulated electrode 100 can be applied to the corresponding body surface of the patient's torso tumor to perform electric field therapy on the tumor. The insulated electrode 100 includes a flexible backing member 12, an electrical functional assembly 11 adhered to the flexible backing member 12, a support member 13 adhered to the backing member 12, an adhesive member 14 adhered to the support member 13, and the wire 15 electrically connected to the electrical functional assembly 11. The insulated electrode 100 of the present disclosure is attached to the corresponding body surface of the patient's tumor site through the backing member 12, and an alternating electric field is applied to the patient's tumor site through the electrical functional assembly 11 to interfere with or prevent the mitosis of the patient's tumor cells, thereby achieving the goal of treating tumors.

Referring to FIG. 7, the electrical functional assembly 11 includes a flexible circuit board 111, a plurality of insulating plates 112 and a plurality of dielectric elements 113 respectively arranged on opposite sides of the flexible circuit board 111, and a plurality of electrical components fixed on the flexible circuit board 111. temperature sensor 114. The temperature sensor 114 is located on the same side of the flexible circuit board 111 as the dielectric element 113. A plurality of dielectric elements 113 are arranged on the side of the flexible circuit board 111 close to the patient's body surface, and a plurality of insulating plates 112 are arranged on the side of the flexible circuit board 111 away from the patient's body surface. The electrical functional assembly 11 are closely attached to the backing member 12 by pasting corresponding parts of the insulating board 112 and the flexible circuit board 111 to the backing member 12. The insulated electrode 100 applies an alternating electrical signal generated by an electric field generator (not shown) to the patient's tumor site through a plurality of dielectric elements 113 arranged on the flexible circuit board 111, so as to perform electric field therapy on the patient's tumor site.

The flexible circuit board 111 includes a plurality of main body portions 1111 arranged in an array, a plurality of connecting portion 1112 between adjacent main body portions 1111, and a wiring portion 1113 electrically connected to the wire 15. The wiring portion 1113 may be laterally extended from a connecting portion 1112, or may be laterally extended from a main body portion 1111 with one free end. The plurality of dielectric elements 113 are arranged in one-to-one correspondence with the plurality of main body portions 1111. The dielectric element 113 is welded on the corresponding main body portion 1111. The main body portion 1111 is disposed at the end of the connecting portion 1112. The main body portion 1111 is extended from the end of the connecting portion 1112. Each main body portion 1111 is at least connected to two adjacent main body portions 1111 through the connecting portion 1112. The main body portion 1111 is generally arranged in a circular sheet shape. Optionally, the main body portion 1111 may also be configured in a strip shape or a belt shape, and integrally formed with the connecting portion 1112. The side of the main body portion 1111 facing the dielectric element 113 is provided with a conductive plate 1114 for soldering (not shown) with the dielectric element 113 to assemble the dielectric element 113 on the main body portion 1111 of the flexible circuit board 111. The center of the conductive plate 1114 coincides with the center of the main body portion 1111. Each conductive plate 1114 has four conductive cores 1115 protruding or exposed from the main body portion 1111. The conductive cores 1115 are arranged in a center-symmetrical shape, which can effectively prevent the position displacement of the dielectric element 113 caused by the stacking of solder (not shown) during the welding process. The four conductive cores 1115 are arranged at intervals, which can reduce the amount of copper foil used to manufacture the conductive cores 1115 and reduce material costs. At the same time, it can also save the amount of solder (not shown) used for welding the conductive cores 1115 and the dielectric element 113, to further reduce material costs.

The four conductive cores 1115 of the same conductive plate 1114 are all petal-shaped. Each conductive core 1115 includes an inner are (not numbered) and an outer arc (not numbered) connected end to end. The inner are (not numbered) and the outer arc (not numbered) of the conductive core 1115 are symmetrically arranged. The inner arcs (not labeled) of the four conductive cores 1115 of the same conductive plate 1114 are all recessed toward the center of the conductive plate 1114. The outer arcs (not labeled) of the four conductive cores 1115 of the same conductive disk 1114 all protrude away from the center of the conductive disk 1114. The four conductive cores 1115 constituting the conductive plate 1114 are arranged symmetrically to the center and axisymmetrically, and each conductive core 1115 is also arranged axisymmetrically, so that the four conductive plates 1114 of the main body portion 1111 are electrically conductive. When the conductive core 1115 is welded with the dielectric element 113, ensure the stress balance of each welding point, ensure the overall welding balance of the dielectric element 113, improve the welding quality, and avoid the unbalanced welding stress causing the dielectric element 113 to tilt, causing the welding joint on the side with a larger distance between the dielectric element 113 and the main body portion 1111 to be weak and prone to breakage. At the same time, it can avoid affecting the bonding degree of the insulated electrode 100. The outer arcs (not numbered) of the four conductive cores 1115 of the same conductive plate 1114 are generally located on the same circumference.

The number of main body portions 1111 is at least 10, and the number of dielectric elements 113 is also at least 10. The arrangement is consistent with the arrangement of the main body portions 1111, and they are distributed in an array area arranged in at least four rows and three columns. The coverage area of the insulated electrode 100 can be increased, the electric field intensity applied to the tumor site for tumor electric field therapy can be enhanced, the range of the alternating electric field covering the tumor site can be increased, and the treatment effect can be improved. The main body portions 1111 are arranged at intervals, and an open space 116 between a plurality of adjacent main body portions 1111 is formed to allow the skin corresponding to the body surface of the patient's tumor part covered by the insulated electrode 100 to breathe freely. The wiring portion 1113 is partly located between the plurality of main parts 1111 and partly disposed in the open space 116 surrounded by the plurality of main parts 1111, which can avoid the overall size of the flexible circuit board 111 from being too large, resulting in increased manufacturing costs.

The main body portion 1111 is in the shape of a circular plate with a diameter of 21-22 mm. The distance between the main body portions 1111 in adjacent rows is at least 1 mm, and the distance between the main body portions 1111 in adjacent columns is at least 1 mm. That is, the distance between the main body portions 1111 located in adjacent columns in the same row is at least 1 mm, and the distance between two adjacent main body portions 1111 in the same row is also at least 1 mm. The distance between the main body portions 1111 located in adjacent rows is at least 1 mm, and the distance between two adjacent main body portions 1111 located in the same row is also at least 1 mm. The distance between the electrode units and the main body portions 1111 in the same row is at least 23 mm, and the distance between the main parts 1111 in the same row is also at least 23 mm.

Preferably, the number of the main body portions 1111 and the number of the dielectric elements 113 is 13, and they may be distributed in a matrix area of five rows and three columns, or may be distributed in a matrix area of five rows and five columns. From the perspective of row arrangement, each of the first row and the last row is provided with two main parts 1111, and each of the middle three rows is provided with three main parts 1111. In this embodiment, the main body portion 1111 is distributed in an array area arranged in five rows and five columns. From the perspective of column arrangement, there are three columns in each of the first column, the third column, and the fifth column. The main body portion 1111, two main body portions 1111 are provided in each of the second row and the fourth row. Specifically, the two main body portions 1111 in the first row are respectively located in the second column and the fourth column, the three main body portions 1111 in each of the middle three rows are respectively located in the first column, the third column, and the fifth column. The two main body portions 1111 are respectively located in the second row and the fourth row. Two adjacent main body portions 1111 in each row are arranged in an alternate column. The main body portions 1111 located in the second column and the fourth column are arranged in alternate rows. Two adjacent main body portions 1111 located in the first column, the third column, and the fifth column are arranged in adjacent rows. The distance between two adjacent main body portions 1111 in a row is equal. The distance between two adjacent main body portions 1111 in the same column is equal. The two main body portions 1111 in the last row are arranged in a disconnected shape, forming a gap 1C between the two main body portions 1111. The wiring portion 1113 is laterally extended from the main body portion 1111 located in the fourth row and third column. The wiring portion 1113 passes through the gap 1C formed between the two main body portions 1111 in the last row.

All the main body portions 1111 of the flexible circuit board 111 can be arranged at intervals in an area with a minimum length of 109 mm and a minimum width of 109 mm. That is, the minimum size of the covering of the main body portion 1111 is about 109 mm×109 mm. The diameter of the main body portion 1111 is 21 mm. The distance between two adjacent main body portions 1111 in each row of the main body portions 111 is the same, at least 23 mm. The distance between two adjacent main body portions 1111 in the second row and the fourth row is the same, at least 67 mm. The distance between two adjacent main body portions 1111 in each of the first row, the third row and the fifth row is the same, at least 1 mm. The aforesaid insulated electrode 100 with the main body portion 1111 having the smallest size is suitable for use by small children.

In order to prevent the electrical functional assembly 11 from overlapping and affecting the treatment effect after the insulated electrode 100 is pasted on the corresponding body surface of the patient's tumor site, the length of the main body portion 1111 is at most 219 mm, and the width at most is 163 mm. That is, all the main parts 1111 of the flexible circuit board 111 are distributed at intervals within a maximum area of 219 mm×163 mm. The diameter of the main body portion 1111 is 22 mm. The maximum distance between the main body portions 1111 of the flexible circuit board 111 in adjacent rows is about 28 mm, and the maximum distance between the main body portions 1111 of the flexible circuit board 111 in adjacent columns is about 50 mm. That is, the maximum distance between two main body portions 1111 in adjacent rows in the same column is about 28 mm, and the maximum distance between two main body portions 1111 in adjacent rows in the same row is 50 mm. The maximum size of the aforementioned flexible circuit board 111 can be suitable for most adult patients. If the patient's waist is too large, two pairs of insulated electrodes 100 can be applied horizontally on the patient's waist for a week. If the waist circumference of the patient is relatively small, two pairs of insulated electrodes 100 can be applied longitudinally on the waist of the patient for one week. If the patient's waist is appropriate, one pair of insulated electrodes 100 can be applied to the patient's waist horizontally, another pair of insulated electrodes 100 can be applied to the patient's waist longitudinally, and two pairs of insulated electrodes 100 can be applied to the patient's waist for one week.

The connecting portion 1112 connects with two adjacent main body portions 1111, and the conductive plate 1114 is disposed on the main body portion 1111 at the end of the connecting portion 1112. The connecting portion 1112 includes a first connecting portion 1112A connecting two adjacent main body portions 1111 located in an alternate row of the same row, a second connecting portion 1112B connecting two main body portions 1111 located in the same column and adjacent rows, and a third connecting portion 1112C connecting the two main body portions 1111 located in adjacent rows and adjacent columns and distributed diagonally. The first connecting portion 1112A is located between two adjacent main body portions 1111 in every alternate row and has the same length. The length of the first connecting portion 1112A is approximately 23 mm-50 mm. The second connecting portion 1112B is located between two adjacent main body portions 1111 in the first row, the third row and the fifth row, and has the same length. The length of the second connecting portion 1112B is approximately 1 mm-28 mm. The length of the third connecting portion 1112C is greater than half of the length of the first connecting portion 1112A. The length of the third connecting portion 1112C is greater than the length of the second connecting portion 1112B. Both the first connecting portion 1112A and the second connecting portion 1112B are substantially arranged in a “˜” shape. The third connecting portion 1112C is generally arranged in an “L” shape or an inclined “˜” shape.

There are 8 third connecting portions 1112C, which are respectively located between the two main body portions 1111 in the second column of the first row and the second row of the first column, and between the two main body portions 1111 in the second column of the first row and the third column of the second row, between the two main body portions 1111 located in the third column of the second row and the fourth column of the first row, between two main body portions 1111 located in the fourth column of the first row and the fifth column of the second row, between the second column and the fifth column of the last row, between the two main body portions 1111 in the first column and the fourth row, between the two main body portions 1111 in the second column in the last row and the third column in the fourth row, between the two main body portions 1111 in the third column and the fourth row and the fourth column in the last row, between the two main body portions 1111 located in the fourth column of the last row and the fifth column of the fourth row. Preferably, the length of the first connecting portion 1112A is greater than the diameter of the main body portion 1111. The length of the second connecting portion 1112B is smaller than the diameter of the main body portion 1111. The first connecting portion 1112A and the second connecting portion 1112B are vertically arranged, the third connecting portion 1112C and its adjacent first connecting portion 1112A are both arranged at an acute angle, and the second connecting portion 1112B and its adjacent third Both connecting portions 1112C are also provided in an acute angle.

The main body portion 1111 can be divided into a peripheral main body portion 1111A located on the periphery of the array and a central main part 1111B surrounded by the peripheral main body portion 1111A and located in the inner layer of the array according to its distribution position in the array. Specifically, there are 10 peripheral main body portions 1111A, and three central main body portions 1111B are located in the same row. The peripheral main body portion 1111A and the central main body portion 1111B are connected two by two through the connecting portion 1112. Two adjacent peripheral body parts 1111A are electrically connected through the first connecting portion 1112A, or through the second connecting portion 1112B, or through the third connection portion 11120. Specifically, the two peripheral main body portions 1111A adjacent to each other in the same column are connected by the second connecting portion 1112B, and the two peripheral body portions 1111A adjacent to each other in the same row are connected by the first connecting portion 1112A, and are located in adjacent rows and adjacent columns and are at opposite corners. Two adjacent peripheral main body portions 1111A arranged in a shape are connected by a third connecting portion 1112C. The peripheral body portion 1111A and the first connection portion 1112A, the second connection portion 1112B, and the third connection portion 1112C located between two adjacent peripheral body portions 1111A are roughly arranged in an octagonal shape with one end open. The peripheral body part 1111A is arranged in an axisymmetric shape, and its axis of symmetry coincides with the straight line where the three central body portions 1111B are located.

The central main body portion 11118 is three body parts 1111 located in the third column. Each central main body portion 1111B and its adjacent peripheral main body portions 1111A are connected either through the first connecting portion 1112A or through the third connecting portion 1112C. The two adjacent central body parts 1111B are electrically connected through the second connecting portion 1112B. Specifically, the central main body portion 1111B is electrically connected to its adjacent peripheral main body portion 1111A in the same line through the first connecting portion 1112A, and the central main body portion 1111B is located in adjacent rows and adjacent columns and arranged diagonally. The peripheral main body portion 1111A is electrically connected through the third connecting portion 1112C, so that the central main part 1111B and its adjacent peripheral main body portion 1111A are connected through at least two connecting portions 1112, ensuring that the peripheral main part The position between 1111A and the central main body portion 1111B is relatively fixed, and the connection is stable, which is convenient for soldering the dielectric element 113 on the flexible circuit board 111. That is to say, the central body part 1111B in the third row is only connected to the peripheral body part 1111A in the same line through the first connecting portion 1112A, which is arranged in a diagonal shape with the adjacent peripheral body parts in adjacent rows and adjacent columns. The portions 1111A are provided in a disconnected shape. Each of the remaining two central body parts 1111B is not only connected to the peripheral body parts 1111A located in adjacent rows and adjacent columns and arranged diagonally through the third connecting portion 1112C, but also connected to the peripheral body parts 1111A located in the adjacent row and column through the first connecting portion 1112A.

Preferably, as shown in FIG. 9, the main body portion 1111 of the flexible circuit board 111 is distributed at intervals in an area of 165 mm×109 mm. The diameter of the main body portion 1111 is 21 mm. The distance between the main body portions 1111 in adjacent rows is 15 mm, and the distance between the main body portions 1111 in the same row and alternate columns of the flexible circuit board 111 is 23 mm. That is, the distance between two main body portions 1111 in adjacent rows in the same column is 15 mm, and the distance between two main body portions 1111 in adjacent rows in the same row is 23 mm. That is, the length of the second connecting portion 1112B is about 15 mm, and the length of the first connecting portion 1112A is about 23 mm. The width of the connection portion 113 is 8 mm. The widths of the first connecting portion 1112A, the second connecting portion 1112B, and the third connecting portion 1112C are all 4.5 mm. The open space 116 formed between the plurality of main body portions 1111 of the flexible circuit board 111 has different areas. In this embodiment, in all the open spaces 116, the nine main body portions 1111 located in the middle three rows are jointly surrounded by four main body portions 1111 in the same column and adjacent rows. The open space 116 has the largest area with an area of about 1065 mm². The area of the open space 116 surrounded by every three main body portions 1111 located in the first row and its adjacent row, the last row and its adjacent row of the flexible circuit board 111 is the smallest. Specifically, the area of the open space 116 formed by the two main body portions 1111 in the same row and the two main body portions 1111 in the same row and the two main body portions 1111 in the spaced row and arranged in a diagonal shape is the smallest. The area is about 470 mm².

The wiring portion 1113 is partly located between the plurality of main parts 1111 and partly disposed in the open space 116 surrounded by the plurality of main parts 1111, so as to prevent the overall size of the main part 111 from being too large and increase the manufacturing cost. The wiring portion 1113 is laterally extended from one of the two main body portions 1111 at the end of the three main body portions 1111 in the third column. Specifically, the wiring portion 1113 is laterally extended from the main body portion 1111 in the fourth row and third column. The wiring portion 1113 is extended from the central body portion 1111B located at the end to the area away from the array of the body portions 1111. The wiring portion 1113 is located between the two third connecting portions 11120, and simultaneously connects with the two third connecting portions 1112C to the central main body portion 1111B at the end. The wiring portion 1113 and the two third connecting portions 1112C connected to the same central body portion 1111B are generally arranged in an arrow shape. The wiring portion 1113 extends out and is located between the two peripheral main portions 1111A arranged in a row and in a disconnected shape. The wiring portion 1113 is substantially perpendicular to the first connecting portion 1112A. The wiring portion 1113 is substantially parallel to the second connecting portion 1112B. The wiring portion 1113 is arranged roughly in the shape of a “one”. The width of the wiring portion 1113 is at least 4 mm. Preferably, the width of the wiring portion 1113 is 4 mm-8 mm. The included angle between the wiring portion 1113 and the third connecting portion 11120, which are simultaneously connected to the same main body portion 1111, is an acute angle. In other embodiment, the wiring portion 1113 can also be extended laterally from the main body portion 1111 or the central main part 1111B located in the second row and third column; and the two main body portions 1111 located in the first row are disconnected. The wiring portion 1113 passes through the gap between the two main body portions 1111. In other embodiment, the wiring portion 1113 can also be arranged laterally by a second connecting portion 1112B located between two adjacent central main body portions 1111B, and the wiring portion 1113 and the second connecting portion 1112B are vertically arranged. The portion 1113 and the second connecting portion 1112B extending from the wiring portion 1113 are arranged in a substantially “T” shape.

The insulating plate 112 is generally arranged in a circular sheet shape. The insulating plate 112 is made of insulating material, and it is adhered to the side of the main body portion 1111 of the flexible circuit board 111 away from the patient's body surface through a sealant (not shown). A flat welding plane is provided for the welding operation between the conductive plate 1114 and the dielectric element 113, thereby improving product yield. The insulating plate 112 can isolate the water vapor in the air from the side of the electrical functional assembly 11 away from the patient's body surface from entering the electrical functional assembly 11, thereby avoiding the contact between the water vapor and the solder (not shown) between the dielectric element 113 and the main body portion 1111, thereby affecting the electrical connection between the main body portion 1111 and the dielectric element 113. The insulation board 112 is provided in one-to-one correspondence with the main body portion 1111, and its arrangement is consistent with that of the main body portion 1111.

The dielectric element 113 is arranged in a circular sheet shape. The dielectric element 113 is made of high dielectric constant material, which can ensure the safety of human body because of its characteristic of blocking direct current and alternating current. The dielectric element 113 has a dielectric constant greater than at least 1000. An annular metal layer 1131 is attached on the side of the dielectric element 113 facing the main body portion 1111, which can be welded to the conductive plate 1114 on the main body portion 1111 by soldering (not shown). The gap (not shown) formed by welding between the dielectric element 113 and the main body portion 1111 is filled with sealant (not shown) to protect the solder (not shown) between the dielectric element 113 and the main body portion 1111 to prevent the dielectric element 113 from being affected by an external force and cause the weld to break, thereby causing the alternating electric field to be unable to be applied to the patient's tumor site through the dielectric element 113. At the same time, it can also prevent water vapor in the air from entering the gap (not shown) to corrode the dielectric solder (not shown) between the element 113 and the main body portion 1111 that affects the electrical connection between the dielectric element 113 and the main body portion 1111.

The space between the outer ring of the metal layer 1131 and the outer edge of the dielectric element 113 can prevent the solder (not shown) disposed between the metal layer 1131 of the dielectric element 113 and the main body portion 1111 from forming a gap when it is heated and melted. The main body portion 1111 overflows, so as to prevent the direct current not hindered by the dielectric element 113 from directly acting on the patient's body surface when the insulated electrode 100 is attached to the corresponding body surface of the patient's tumor site. The dielectric element 113 has a through hole 1132 disposed therethrough for accommodating the temperature sensor 114. The edge of the through hole 1132 of the dielectric element 113 and the inner ring of the metal layer 1131 of the dielectric element 113 are arranged at intervals, which can avoid soldering (not shown) between the metal layer 1131 of the dielectric element 113 and the main body portion 1111. When heated and melted, it diffuses toward the through hole 1132 of the dielectric element 113 to cause a short circuit in the temperature sensor 114. The main body portion 1111, the insulating plate 112 and the dielectric element 113 are provided in one-to-one correspondence, and the centers of the three are located on the same straight line. The arrangement of the insulating plates 112 and the dielectric elements 113 is consistent with that of the main body portion 1111, and they are all distributed in an array area arranged in five rows and five columns.

The main body portion 1111 of the flexible circuit board 111, the insulating plate 112 arranged on the side away from the patient's body surface of the main body portion 1111 of the flexible circuit board 111, and the dielectric element 113 arranged on the side of the main body portion 1111 of the flexible circuit board 111 facing the patient's skin Together they form the electrode unit 110 of the electrical functional assembly 11. The arrangement of the electrode units 110 of the electrical functional assembly 11 is consistent with the arrangement of the main body portion 1111 of the flexible circuit board 111. The connecting portion 1112 is located between two adjacent electrode units 110.

The temperature sensor 114 is fixed on the main body portion 1111 and is used to monitor the temperature of the sticker 14, so as to monitor the temperature of the human skin attached to the sticker 14. When the temperature detected by the temperature sensor 114 exceeds the upper limit of the safe temperature of the human body, the electric field generator (not shown) can reduce or shut down the alternating current transmitted to the insulating electrode 100 in time to avoid low-temperature burns on the human body. The temperature sensor 114 is welded to the main body portion 1111 and then sealed with a sealant (not shown), so as to prevent water vapor from corroding the temperature sensor 114 and causing the temperature sensor 114 to fail. The temperature sensor 114 is disposed on a peripheral main body portion 1111 among the plurality of main body portions 1111 arranged in an array. That is, the temperature sensor 114 is disposed on the peripheral body portion 1111A.

One end of the wire 15 is welded to the wiring portion 1113 of the electrical functional assembly 11, and the other end is provided with a plug (not labeled) electrically connected to the electric field generator (not shown). The plug (not labeled) of the wire 15 can be directly plugged into the electric field generator (not shown), or can be plugged into the adapter (not shown) of the tumor electric field therapy system, and then passed through the adapter (not shown) which is electrically connected with the electric field generator (not shown) to realize the electrical connection between the wire 15 and the electric field generator (not shown). The connection between the wire 15 and the wiring portion 1113 on the flexible circuit board 111 is covered with a heat-shrinkable sleeve 151, which is used to seal and insulate the connection between the wire 15 and the wiring portion 1113 on the flexible circuit board 111, and improve the strength support, to avoid breakage at the connection between the wire 15 and the electrical functional assembly 11, and at the same time prevent dust and water.

The supporting member 13 is arranged in a sheet shape. A plurality of support members 13 are provided. The supporting member 13 is adhered on the backing member 12 in a manner of surrounding the electrode units 110 arranged in a row. A plurality of support members 13 are arranged at intervals. The supporting member 13 has a plurality of through holes 131 corresponding to the corresponding electrode units 110. A plurality of through holes 131 are arranged at intervals. The thickness of the support member 13 is basically the same as the thickness of the electrode unit 110, and the plane where the top of the support member 13 is located is at the same vertical height as the surface of the electrode unit 110 facing the patient's body surface, that is, the support member 13 is close to the patient's body surface. The side surface is flush with the surface of the dielectric element 113 close to the patient's body surface, so that the adhesive part 14 can be evenly covered on the support part 13 and the electrode unit 110, and the comfort of the insulated electrode 100 is improved. The support member 13 can be made of polyethylene (PE) material or PET material or heat-conducting silica gel sheet or composited by polyurethane, polyethylene, dispersant, flame retardant, carbon fiber, etc. It is soft, stable in chemical properties, light in weight and not easy to deform. Preferably, the support member 13 is flexible foam.

The sticker 14 is arranged in a sheet shape, one side of which is attached to the support member 13 and the dielectric element 113, and the other side is attached to the body surface of the patient. The paste 14 is a conductive hydrogel, which can be used as a conductive medium to conduct the alternating current passing through the dielectric element 113 to the patient's tumor site. The number of sticking pieces 14 is the same as the number of supporting pieces 13. The size of the sticking part 14 is substantially the same as that of the supporting part 13.

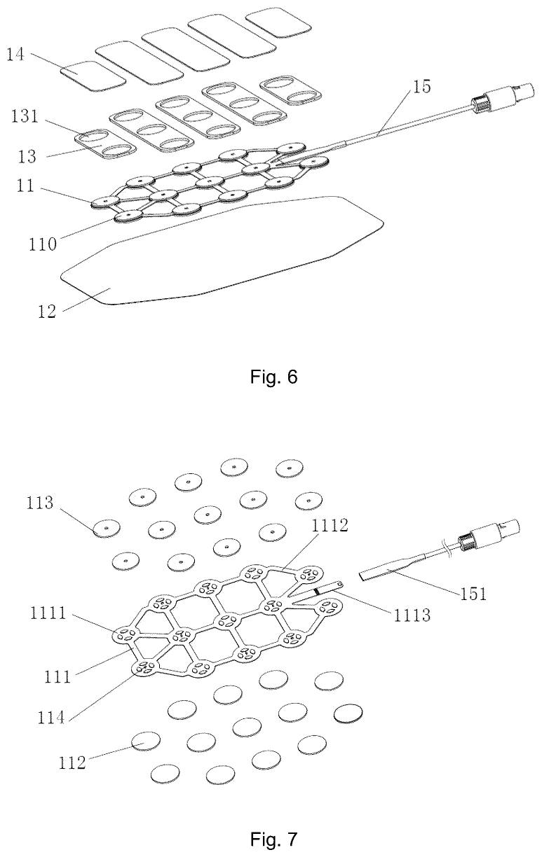

As shown in FIG. 6, the backing member 12 is arranged in sheet form, which is mainly made of compatible flexible, breathable, insulating and sterilizable materials. The backing member 12 has a plurality of ventilating holes (not shown) that run through the setting, which can make the hair follicles and sweat glands of the skin covered by the backing member 12 on the patient's body surface can breathe freely when the backing member 12 is applied on the patient's body surface. The sweat glands and hair follicles on the patient's body surface covered by the backing member 12 damage the superficial layer of the patient's skin due to blockage and cause skin inflammation. The backing member 12 is a mesh fabric. Specifically, the backing member 12 is a mesh non-woven fabric. The side of the backing member 12 facing the patient's body surface is also coated with a material-compatible adhesive (not shown), which is used to closely fit the backing member 12 to the body surface of the patient's target area.

The main body portions 1111 are arranged at intervals, and an open space 116 between a plurality of adjacent main body portions 1111 is formed to allow the patient's tumor part covered by the insulated electrode 100 to the skin corresponding to the body surface breathes freely.

### Variation of the First Embodiment of the Insulated Electrode

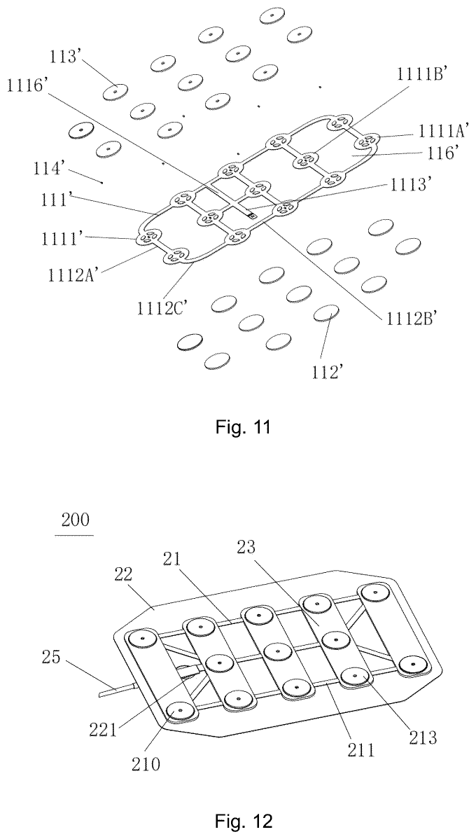

FIGS. 10 to 11 show the insulated electrode 100′ in this embodiment, which is also applied on the body surface of the patient's torso, and is used to perform tumor electric field therapy on the tumor site located in the torso, and it also includes a flexible backing member 12′, an adhesive, an electrical functional assembly 11′ disposed on the backing member 12′, a support member 13′ adhered to the backing member 12′, a sticking part (not shown) adhered to the support member 13′ and an electrical functional assembly 11, an electrically connected wire 15′. The electrical functional assembly 11′ also includes a flexible circuit board 111′, a plurality of insulating plates 112′ and a plurality of dielectric elements 113′ respectively arranged on opposite sides of the flexible circuit board, and a plurality of temperature sensors 114′ fixed on the flexible circuit board 111′. The main body portion 1111′ of the flexible circuit board 111′, the dielectric element 113′ and the insulating plate 112′ are arranged in a one-to-one correspondence, and constitute the electrode unit 110′ of the electrical functional assembly 11′. The main body portions 1111′ are also arranged in five rows and five columns, and their positions in the array area of five rows and five columns are consistent with the arrangement of the main body portions 1111 in the first embodiment.

The difference between the insulated electrode 100′ in this embodiment and the insulated electrode 100 in the first embodiment is that the peripheral body portion 1111A′ of the flexible circuit board 111′ of the electrical function component 11′ of the insulated electrode 100′ passes through the connecting portion 1112′ connected two by two. The central main body portion 1111B′ is only connected with its adjacent peripheral main body portion 1111A′ in the same row. Specifically, two adjacent peripheral main body portions 1111A′ are connected in pairs through the first connecting portion 1112A′, or connected in pairs through the second connecting portion 1112B′, or connected in pairs through the third connecting portion 1112C′. The peripheral body part 1111A′ and the first connecting portion 1112A′, the second connecting portion 1112B′ and the third connection portion 1112C′ between two adjacent peripheral body parts 1111A′ are roughly racetrack-shaped. The central body part 1111B′ is connected to the peripheral body parts 1111A′ located in the same line through the first connecting portion 1112A′. The central body part 1111B′ is arranged in a disconnected shape from the peripheral body parts 1111A′ located in adjacent rows and adjacent columns and arranged diagonally. Two adjacent central main body portions 1111B′ of the three central main body portions 1111B′ are arranged in a disconnected shape. There is no second connecting portion 1112B′ between two adjacent central main body portions 1111B′ arranged in a disconnected shape. The third connecting portion 1112C′ is arranged in an arc shape. There are four third connecting portions 1112C′, which are respectively located between the two peripheral main parts 1111A′ in the second column of the first row and the second row of the first column, and between the two peripheral main parts in the fourth column of the first row and the fifth column of the second row between the two peripheral main parts 1111A′, between the second column of the last row and the fourth row of the first column, and between the two peripheral main parts 1111A′ of the fourth column of the last row and the fifth column of the fourth row. Both the third connecting portion 1112C′ and the first connecting portion 1112A′ adjacent thereto are substantially obtuse or acute angled. Both the third connecting portion 1112C′ and the adjacent second connecting portion 1112B′ are arranged in an obtuse angle. The peripheral body part 1111A′ has the same diameter as the central body part 1111B′, and the length of the second connecting portion 1112B′ is slightly longer than the diameter of the peripheral body part 1111A′.

The wiring portion 1113′ is laterally extended from a second connecting portion 1112B′. Specifically, the wiring portion 1113′ is laterally extended from the second connecting portion 1112B′ located between two adjacent central main body portions 1111B′. The wiring portion 1113′ and the second connecting portion 1112B′ extending from the wiring portion 1113′ are generally arranged in a “T” shape. The wiring portion 1113′ is arranged perpendicular to the second connecting portion 1112B′. The wiring portion 1113′ is substantially parallel to the first connecting portion 1112A′.

The flexible circuit board 111′ also has a reinforcing part 1116′ opposite to the wiring portion 1113′, which can provide traction for the wiring portion 1113′ and avoid uneven force when the insulated electrode 100′ is attached to the body surface of the patient's tumor. However, the sticking of the insulated electrode 100′ is affected. Specifically, the reinforcing portion 1116′ is extended from the second connecting portion 1112B′ extending laterally from the wiring portion 1113′. The reinforcing part 1116′ and the wiring portion 1113′ are respectively located on opposite sides of the second connecting portion 1112B′ connected to the wiring portion 1113′. One end of the reinforcing portion 1116′ is connected to the second connecting portion 1112B′ connected to the wiring portion 1113′, and the other end is connected to the second connecting portion 1112B′ adjacent to the second connecting portion 1112B′ and located between two adjacent peripheral main parts 1111A′. The reinforcing part 1116′ is bridged between two adjacent second connecting portions 1112B′ arranged in parallel. The reinforcing part 1116′, the wiring portion 1113′ and the second connecting portion 1112B′ connected to the wiring portion 1113′ are arranged in a substantially “cross” shape.

The backing member 12′ is provided with a wire hole 121′ corresponding to the wiring portion 1113′ of the flexible circuit board 111′. One end of the wire 15′ is electrically connected to the wiring portion 1113′ through the wire hole 121′. The wire 15′ extends into the flexible circuit board 111′ from the side of the backing member 12′ to connect with the wiring portion 1113′, avoiding a large number of wires 15′ being directly pressed on the patient's epidermis, resulting in comfortable application of the insulated electrode 100′.

The main body portions 1111′ are arranged at intervals, and an open space 116′ between a plurality of adjacent main body portions 1111′ is formed to allow the insulated electrode 100′ to the covered patient's tumor site corresponds to the free breathing of the skin on the body surface. The dimensions of the main body portion 1111′, the connection portion 1112′, the wiring portion 1113′ and the size of the open space 116′ of the electrical functional assembly 11′ will be described in detail below.

All the main parts 1111′ of the flexible circuit board 111′ are spaced apart in an area with a minimum length of 193 mm and a minimum width of 109 mm. That is, the minimum size of the flexible circuit board 111′ is 193 mm×109 mm. The distance between the main body portions 1111′ in adjacent rows in the flexible circuit board 111′ is greater than the diameter of the main body portions 1111′, at least 22 mm. That is, the length of the second connecting portion 1112B′ is greater than the diameter of the main body portion 1111′, and is at least 22 mm. The third connecting portion 1112C′ is arranged in an arc shape. The length of the third connecting portion 1112C′ is at least 26.7 mm. The width of the reinforced part 1116′ is 4.5 mm-6 mm.

Among all the open spaces 116′, the open space 116′ formed by the five main body portions 1111′ between the first row and the second row has the largest area. The area of the open space 116′ formed by the two main body portions 1111′ and receiving the reinforcing part 1116′ is the smallest.

The insulated electrodes 100, 100″ of this embodiment apply an alternating electric field to the patient's tumor site through at least 10 electrode units 110, 110″ distributed in the array area of at least four rows and three columns to treat the tumor, which increases the size of the insulated electrode 100, 100″ covering the area of the tumor, which enhances the electric field intensity for tumor electric field therapy, thereby ensuring the effect of tumor electric field therapy. It is suitable for use on the patient's trunk. For example, when it is applied on the abdomen, the front One insulated electrode 100, 100″ is pasted on the side and the back side, and one insulated electrode 100, 100″ is pasted on each side. Depending on the size of the individual, it can also be used with other insulated electrodes. The insulated electrode 100 of this embodiment is more suitable for sticking on the narrower side waist; and the insulated electrodes 100, 100′ have a plurality of open spaces 116, 116′ formed between the main body portions 1111, 1111′ arranged at intervals, which can be insulated. The electrodes 100, 100′ discharge the heat accumulated on the surface of the patient's skin and the water vapor generated by sweating to the air during tumor treatment, so as to avoid skin inflammation caused by sweating and clogged pores of the epidermis of the patient on which the insulated electrodes 100, 100′ are applied, can ensure the long-term application of the insulating electrodes 100, 100′, and ensure the therapeutic effect of the electric field.

### Second Embodiment of Insulated Electrodes

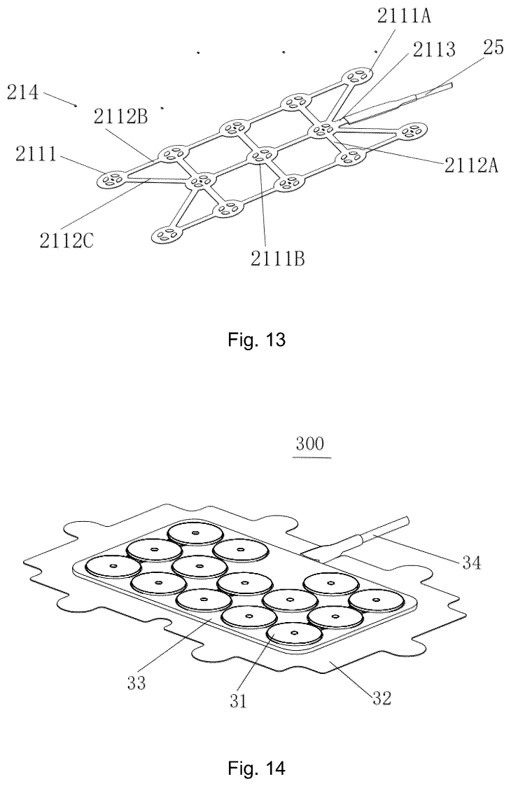

FIG. 12 to FIG. 13 show the insulated electrode 200 of the second embodiment, which is also pasted on the body surface of the patient's torso, and is used to perform tumor electric field therapy on the tumor site located in the torso, and it also includes a flexible backing 22 which is adhered to the electrical functional assembly 21 on the backing 22, a support 23 adhered on the backing 22, a sticker (not shown) adhered to the support 23, and wires 25 electrically connected to the electrical functional assembly 21.

The difference between the insulated electrode 200 in this embodiment and the insulated electrode 100 in the first embodiment is that the electrical function component 21 of the insulated electrode 200 includes a flexible circuit board 211, a plurality of insulating plates (not shown), a plurality of dielectric component 213 and a plurality of temperature sensors 214 disposed on the flexible circuit board 211. Insulation plates (not shown) and dielectric elements 213 are disposed on opposite sides of the main body portion 2111 on the flexible circuit board 211 in one-to-one correspondence, forming a plurality of electrode units 210. The main body portion 2111 is distributed in an array area of five rows and three columns. From the perspective of column arrangement, the first column and the third column each have five main body portions 2111, and the second column has three main body portions 2111. Specifically, the two main body portions 2111 located in the first row are respectively located in the first column and the third column. The two main body portions 2111 located in the bottom row are also located in the first column and the third column, respectively. The three main bodies 2111 in each of the middle three rows are respectively located in the first column, the second column and the third column. The main body portions 2111 located in the first row and the last row are arranged at intervals, and the main parts 2111 located in the first row and the last row are arranged in a disconnected shape. The distance between two adjacent main body portions 2111 in a row is not equal. The distance between two adjacent main body portions 2111 in the same column is equal. The thirteen main body portions 2111 are arranged in an axisymmetric shape, one axis of symmetry coincides with the straight line of the three main body portions 2111 in the third row, and the other axis of symmetry coincides with the straight line of the three main body portions 2111 in the second row. The thirteen main body portions 2111 are also arranged symmetrically to the center, and the center of symmetry coincides with the center of the main body portions 2111 located in the third row and third column. The arrangement of the electrode units 210 is consistent with that of the main body 2111, and is located in an array area of five rows and three columns.

The main body portion 2111 can be divided into 12 peripheral main body portions 2111A located on the periphery of the array and one central main body portion 2111B surrounded by the peripheral main part 2111A and located in the inner layer of the array according to its distribution position in the array. Specifically, one central body part 2111B is the body part 2111 located at the position of the third row and the second column. The 12 peripheral body parts 2111A are all the other body parts 2111 except the body part 2111 located at the third row and the second column. The peripheral body part 2111A is connected either through the second connecting portion 2112B, or through the third connection portion 2112C. The two peripheral main body portions 2111A adjacent in the same column are connected by the second connecting portion 2112B. The two peripheral body parts 2111A located in adjacent rows and adjacent columns and arranged diagonally are connected by a third connecting portion 2112C. The space between two adjacent peripheral main body portions 2111A arranged at intervals in a row is arranged in a disconnected shape. The peripheral body part 2111A is connected to the central body part 2111B either through the first connecting portion 2112A, or through the second connecting portion 2112B. Specifically, the peripheral main body portion 2111A and the central main body portion 2111B adjacent to each other are connected through the first connecting portion 2112A. The peripheral body part 2111A and the central body part 2111B adjacent in the same row are connected through the second connecting portion 2112B. The first connecting portion 2112A is located between the two main body portions 2111 in an adjacent row, and has the same length. The second connecting portion 2112B is located between the two main body portions 2111 in adjacent rows in the same column and has the same length. The length of the third connecting portion 2112C is greater than the length of the first connecting portion 2112A. The number of third connecting portions 2112C is 4, which are respectively located between the two peripheral main parts 2111A in the first column of the first row and the second column of the second row, and between the two peripheral parts of the second column of the second row and the third column of the first row. Between the main body portions 2111A, between the two peripheral main body portions 2111A located in the first column of the fifth row and the second column of the fourth row, and between the two peripheral main body portions 2111A located in the second column of the fourth row and the third column of the fifth row. The peripheral body parts 2111A are arranged in an axisymmetric shape, one axis of symmetry coincides with the extending direction of the row where the central body part 2111B is located, and the other axis of symmetry coincides with the extending direction of the column where the central body part 2111B is located. The connection portion 2113 is extended from the peripheral body portion 2111A located in the fourth row and the second column. The connecting portion 2113 is located between two adjacent third connecting portions 2112C that are connected to the same peripheral body portion 2111A.

The backing 22 is provided with a wiring hole 221 corresponding to the wiring portion 2113 of the flexible circuit board 211. One end of the wire 25 is electrically connected to the wiring portion 2113 through the wire hole 221. The wires 25 protrude into the flexible circuit board 211 from the side of the backing 22 to connect with the wiring portion 2113, avoiding the problem that a large number of wires 25 are directly pressed on the patient's epidermis, resulting in reduced comfort when the insulated electrode 200 is applied.

### Third Embodiment of Insulated Electrodes

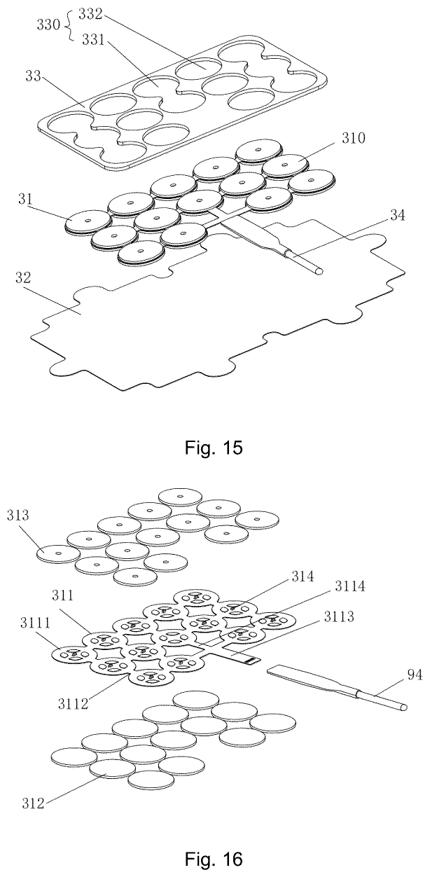

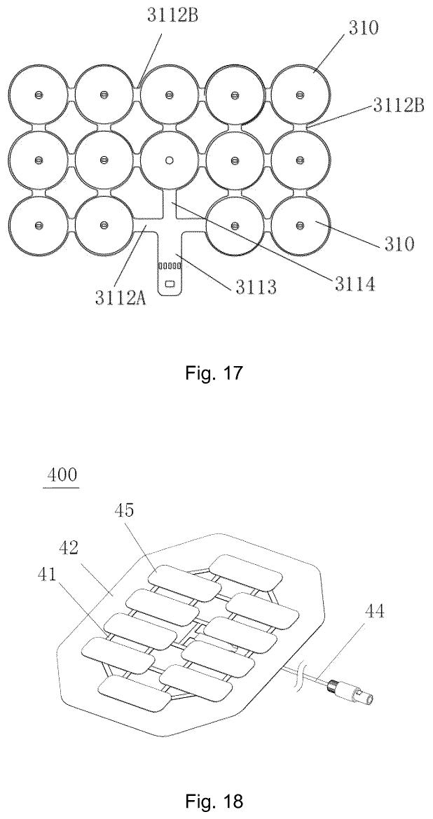

Refer to FIGS. 14 to 17, the insulated electrode 300 of this embodiment includes a backing 32, an electrical functional assembly 31 glued on the backing 32, a support 33 glued on the backing 32, and a backing 32 glued on the backing. The backing 32 is on and covers the supporting member 33 and the corresponding part of the electrical functional assembly 31, and the wire 34 is electrically connected to the electrical functional assembly 31.

The electrical functional assembly 31 includes a plurality of electrode units 310 arranged in a substantially rectangular array, a plurality of connecting portions 3112 located between adjacent electrode units 310 and electrically connecting two adjacent electrode units 310, and extending from a connecting portion 3112. Two adjacent electrode units 310 are connected to each other through the connecting portion 3112, so that the electrical functional assembly 31 form a network structure. The connecting portion 3112 includes a first connecting portion 3112A connecting two adjacent electrode units 310 and the wiring portion 3113 and a plurality of second connecting portions 3112B connecting only two adjacent electrode units 310 in the same row or column. The connection portion 3113 is laterally extended from the first connection portion 3112A in a direction away from the electrode unit 310, and is electrically connected to the wire 34.

The electrode unit 310 includes a main body 3111, an insulating plate 312 disposed on the side of the main body 3111 away from the human skin, a dielectric element 313 disposed on the side of the main body 3111 facing the human skin, and a dielectric element 313 selectively disposed on the main body 3111 and connected to the dielectric. The electrical element 13 is located on the same side as the temperature sensor 314. Wherein, the main body portion 3111, the connection portion 3112 and the connection portion 3113 jointly constitute the flexible circuit board 311 of the electrical functional assembly 31. In this embodiment, the electrode units 310 of the electrical functional assembly 31 are arranged in a denser manner to increase the electric field intensity. The specific structure of the electrode unit 310 is the same as that of the first embodiment, the content of which can be referred to.

The electrical functional assembly 31 includes electrode units 310 arranged in three rows and five columns, and a connecting portion 3112 connecting two adjacent electrode units 310 in the same row or column. There are 14 electrode units 310 in total. From the perspective of row arrangement, the electrode units 310 include five electrode units 310 in the first row, five electrode units 310 in the middle row, and four electrode units 310 in the last row. The connection portion 3112 between two adjacent electrode units 310 in the first row or the middle row has the same length, and is between 1 mm-3 mm, preferably 2.1 mm. The connecting portion 3112 between two adjacent electrode units 310 in the last row has different lengths, wherein the length of the connecting portion 3112 between two adjacent electrode units 310 in adjacent columns in the last row is equal to that the length of the connection portion 3112 between two adjacent electrode units 310 in the middle row. The length of the connection portion 3112 between two adjacent electrode units 310 in the adjacent column in the last row is less than the length of the two adjacent electrode units 310 in the middle row of the last row. The length of the connecting portion 3112 between two adjacent electrode units 310 in the adjacent column in the last row is between 1 mm-3 mm, preferably 2.1 mm. The length of the connecting portion 3112 between two adjacent electrode units 310 in the last row and the spaced column is between 22 mm and 27 mm.

From the perspective of the arrangement of the electrode units 310, there are only two electrode units 310 in the middle column, and three electrode units 310 in the other four columns. The connecting portion 3112 connecting two adjacent electrode units 310 in each column has the same length, which is equal to the length of the connecting portion 3112 connecting two adjacent electrode units 310 in the first or middle row. The length of the connecting portion 3112 connecting two adjacent electrode units 310 in each row is between 1 mm-3 mm, preferably 2.1 mm. The lengths of the connecting portions 3112 between two adjacent electrode units 310 arranged in a row are all the same, between 1 mm-3 mm, preferably 2.1 mm. The lengths of the connection portions 3112 between two adjacent electrode units 310 arranged in a row are different. The length of the connection portion 3112 connecting two electrode units 310 located in adjacent columns in a row is shorter than the length of the connection portion 3112 connecting two electrode units 310 arranged in alternate columns in a row. The connecting portions 3112 between two adjacent electrode units 310 in adjacent rows in the same column are all second connecting portions 3112B. The connecting portion 3112 between two adjacent electrode units 310 in adjacent columns in a row is also the second connecting portion 3112B. The length of the second connecting portion is between 1 mm-3 mm, preferably 2.1 mm. The connecting portion 3112 between two adjacent electrode units 310 located in an alternate column in a row is a first connecting portion 3112A. Both the first connecting portion 3112A and the second connecting portion 3112B are arranged in a “˜” shape. The length of the first connecting portion 3112A is different from the length of the second connecting portion 3112B. The length of the first connecting portion 3112A is greater than the length of the second connecting portion 3112B.

The flexible circuit board 311 of the electrical functional assembly 31 may further include a reinforcing part 3114, one end of the reinforcing part 3114 is connected to the first connecting portion 3112A, and the other end is connected to the electrode unit 310 corresponding to the first connecting portion 3112A. The reinforcing part 3114 is connected to the wiring portion 3113 on the same straight line. The reinforcing portion 3114 is vertically disposed to the first connecting portion 3112A. The reinforcing part 3114 and the first connecting portion 3112A are arranged in an “F” shape or a “T” shape. The reinforcing part 3114 and the connecting portion 3113 are respectively located on opposite sides of the first connecting portion 3112A. The reinforcing part 3114 can strengthen the strength of the connecting portion 3113 arranged opposite to it, and can provide traction for the connecting portion 3113, so as to avoid affecting the application of the insulated electrode 300 due to uneven force when the insulated electrode 300 is applied on the body surface of the patient's tumor site. The length of the reinforcing portion 3114 is not less than the length of the second connecting portion 3112B. That is, the length of the reinforcing part 3114 is greater than or equal to the length of the second connecting portion 3112B connecting two adjacent electrode units 310 in adjacent rows in the same row, or greater than or equal to the length of the second connecting portion 3112B connecting two adjacent electrode units 310 in adjacent rows in the same column.

In this embodiment, the electrode unit 310 is roughly in the shape of a circular sheet, and the diameter of the electrode unit 310 is about 21 mm. The length of the second connecting portion 3112B is 1 mm-3 mm, which can increase the number of electrode units 310 in the unit area of the insulated electrode 300, and can increase the coverage of the electrode unit 310 of the insulated electrode 300 without increasing the overall area of the insulated electrode 300, increase the area of the electric field applied to the tumor site for TTF treatment, increase the range of the alternating electric field covering the tumor site, and improve the therapeutic effect.

In this embodiment, the corresponding part of the connecting portion 3113 close to the connecting portion 3112 is located between the two electrode units 310 in the middle of the last row, so as to use the space between the electrode units 310 to shorten the distance of the connecting portion 3113 beyond the edge of the electrode unit 310, thereby avoiding the overall size of the electrical functional assembly 31, resulting in increased manufacturing costs. The connecting portion 3113 and its adjacent electrode unit 310 are arranged at intervals, which can provide a larger operation space for welding the connecting portion 3113 and the wire 34. To facilitate the laying of conductive traces (not shown), the connection portion 3113 is wider than the connection portion 3112. Preferably, the width of the connecting portion 3112 is 4-6 mm, and the width of the connecting portion 3113 is 7-9 mm. In this embodiment, the width of the connection portion 3112 is 4.5 mm, and the width of the connection portion 3113 is 8 mm. It can be understood that part of the connecting portion 3112 may not be used for laying conductive traces (not shown), and is only used to increase the strength of the flexible circuit board 11.

In this embodiment, the support member 33 is a whole piece of foam. The support member 33 is provided with a plurality of through holes 330 corresponding to the electrode units 310 of the electrical functional assembly 31 for accommodating the corresponding electrode units 310. The supporting member 33 surrounds each electrode unit 310 of the electrical functional assembly 31, which can improve the overall strength of the insulating electrode 300. The through holes 330 include a plurality of first through holes 331 and a plurality of second through holes 332. A plurality of first through holes 331 are arranged in a connected shape, and surround a plurality of electrode units 310 arranged in a row, which can accommodate the connection portion 3112 between two adjacent electrode units 310 in the same row, and reduce the contact between the support member 33 and the electrical connection. The contact of the connecting portion 3112 of the functional component 31 enables the support member 33 to be more smoothly attached to the backing 32. A plurality of second through holes 332 are disposed on the support member 33 at intervals, and respectively surround one electrode unit 310 arranged in a row. In this embodiment, the plurality of first through holes 331 respectively surround the three electrode units 310 in the first column, the two electrode units 310 in the third column, and the three electrode units 310 in the fifth column. A plurality of second through holes 332 surround the respective electrode units 310 in the second row and the fourth row respectively. A plurality of second through holes 332 are arranged in a row, and the plurality of second through holes 332 arranged in a row are arranged at intervals to ensure the strength of the support member 33 itself and avoid being broken by external force. The first through hole 331 is arranged roughly in the shape of a racetrack.

The adhesive part (not shown) is a whole piece, and its size is slightly larger than that of the supporting part 33. The sticker (not shown) is preferably conductive gel. The sticky part (not shown) has double-sided adhesiveness, which can keep the skin surface moist and relieve local pressure when in contact with the skin.

The insulated electrode 300 of this embodiment can also cover the release paper (not shown) on the outside of the sticker (not shown) and the backing 32 to protect the sticker (not shown) and the backing 32 and prevent the sticker from (not shown) and the backing 32 are soiled. The insulated electrode 300 can be covered on the sticker (not shown) and the backing 32 by only one piece of release paper (not shown), or more than two pieces of release paper (not shown) are jointly covered on the sticker (not shown). shown) and backing 32. When in use, the release paper (not shown) is torn off, and the insulated electrode 300 is pasted on the body surface corresponding to the tumor site of the human body.

The insulated electrode 300 of this embodiment applies an alternating electric field to the tumor site of the patient through the 14 electrode units 310 provided thereon to perform tumor treatment, which can avoid insufficient electric field treatment affecting the treatment effect due to differences in tumor size, location, and position, and increase the covering area of the electrode unit 310 of the large insulated electrode 300, increase the intensity of the electric field applied to the tumor site for TTF treatment, increase the range of the alternating electric field covering the tumor site, and improve the treatment effect.

### Fourth Embodiment of Insulated Electrodes

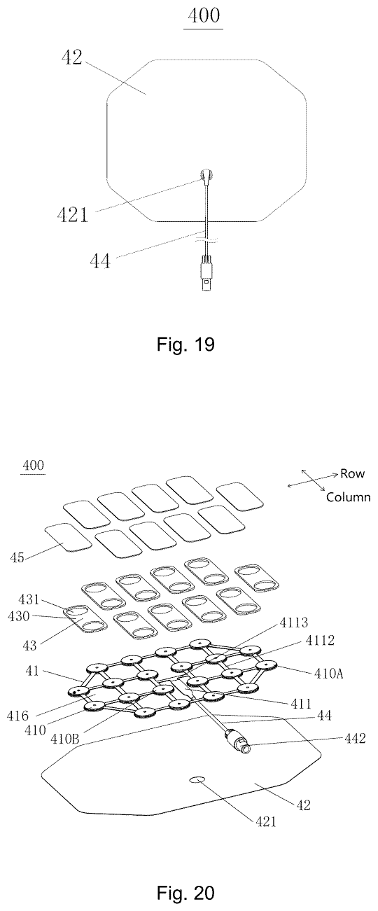

Refer to FIGS. 18 to 21, the insulated electrode 400 of this embodiment includes a backing 42, an electrical functional assembly 41 adhered to the backing 42, a support 43 adhered to the backing 42, an adhesive part 45 covering the support 43 and the corresponding parts of the electrical functional assembly 41, and wires 44 electrically connected with the electrical functional assembly 41. The insulated electrode 400 is attached to the corresponding body surface of the patient's tumor through the backing 42, and an alternating electric field is applied to the patient's tumor through the electrical functional assembly 41 to interfere or prevent the mitosis of the patient's tumor cells, thereby achieving the purpose of treating the tumor.

The electrical functional assembly 41 is arranged in a grid shape, including a plurality of electrode units 410 arranged in an array, a plurality of connecting portions 4112 connecting two adjacent electrode units 410, and a wiring portion 4113 welded to the wire 44. A plurality of electrode units 410 are distributed on the grid points of the electrical functional assembly 41 at intervals. Each electrode unit 410 is connected to at least two adjacent electrode units 410 through a connecting portion 4112. Each electrode unit 410 is connected to at least two connecting portion 4112. There are at least ten electrode units 410, which are distributed in an array area of at least three rows and four columns, which can increase the coverage area of the electrode units 410 of the insulated electrode 400, and enhance the electric field intensity applied to the tumor site for tumor electric field therapy, increase the range of the alternating electric field covering the tumor site, and improve the therapeutic effect.

Preferably, each electrode unit 410 is connected to at least three adjacent electrode units 410 through a connecting portion 4112. Each electrode unit 410 is connected to at least three connecting portion 4112. There are twenty electrode units 410 distributed in an array area of four rows and six columns. The number of electrode units 410 in each column is not exactly the same. The number of electrode units 410 in each row may or may not be completely the same. Among the plurality of electrode units 410, at least one of the adjacent two electrode units 410 is arranged in a disconnected shape, and a gap is formed between the two adjacent electrode units 410 arranged in a disconnected shape and through which the wiring portion 4113 passes. The connecting portion 4113 is laterally extended from the connecting portion 4112 opposite to the space 4C. The connecting portion 4112 extending from the connecting portion 4113 is arranged vertically to the connecting portion 4113, and both of them are arranged in a substantially “T” shape. The connecting portion 4113 is roughly arranged in a “˜” shape. Optionally, the connecting portion 4113 is arranged in a “T” shape, and bridged between two connecting portions 4112 respectively connected to two adjacent electrode units 410 arranged in a disconnected shape. The connecting portion 4113 is located between the plurality of electrode units 410 and is disposed in the space surrounded by the plurality of electrode units 410, which can prevent the overall size of the electrical functional assembly 41 from being too large, resulting in increased manufacturing costs.

Twenty electrode units 410 are arranged in an array area of four rows and six columns in such a manner that two electrode units 410 are arranged in each column, and four electrode units are arranged in each of the other four columns. Specifically, the twenty electrode units 410 are distributed in the array area of four rows and six columns in a manner that four columns of four electrode units 410 are adjacent to each other. The distance between two adjacent electrode units 410 arranged in a row is the same. A plurality of connecting portions 4112 connecting two adjacent electrode units 410 arranged in a row have the same length. Specifically, the electrode units 410 in each of the two columns with only two electrode units 410 are arranged in adjacent rows, and the distance between two adjacent electrode units 410 arranged in a row is the same, and a plurality of electrode units 410 are connected into the connecting portions 4112. Two adjacent electrode units 410 arranged in a row have the same length. The four electrode units 410 in the two columns may be arranged in a row-aligned manner, or may be arranged in a row-wise staggered manner, or one of them may be arranged in a row-directed manner and the other may be arranged in a row-wise staggered manner. Optionally, the two electrode units 410 in at least one of the two columns with only two electrode units 410 are arranged in rows at intervals, the spacing between the electrode units 410 arranged in columns is different, and multiple connections are adjacent to each other in columns. The connecting portions 4112 of the two electrode units 410 have different lengths.

Optionally, at least two of the twenty electrode units 410 among the four columns with four electrode units 410 are distributed in an array area of four rows and six columns at intervals. The distances between two adjacent electrode units 410 arranged in a row are different, and the connecting portions 4112 between the two adjacent electrode units 410 arranged in a row have different lengths. Specifically, only the two electrode units 410 in at least one of the two columns of the two electrode units 410 are arranged in rows at intervals, the distances between adjacent two electrode units 410 arranged in columns are different, and a plurality of electrode units 410 are connected in rows. The connecting portion 4112 between two adjacent electrode units 410 of the cloth has different lengths. Optionally, only when the two electrode units 410 in each of the two columns of the two electrode units 410 are arranged in adjacent rows, the distance between the adjacent two electrode units 410 arranged in a row is the same, and a plurality of connecting portion 4112 connecting two adjacent electrode units 410 arranged in a row has the same length.

Optionally, twenty electrode units 410 are distributed in an array area of four rows and six columns in such a way that one electrode unit 410 is arranged in one column, three electrode units 410 are arranged in one column, and four electrode units 410 are arranged in each of the remaining four columns. Specifically, twenty electrode units 410 are distributed in four rows in such a way that one electrode unit 410 is provided in the first column, four electrode units 410 are provided in each of the middle four columns, and three adjacent electrode units 410 are provided in the last column. In the six-column array area, the distances between two adjacent electrode units 410 arranged in rows are the same, and the distances between two adjacent electrode units 410 arranged in columns are the same. That is to say, the connecting portions 4112 between two adjacent electrode units 410 connected in a row have the same length. A plurality of connecting portions 4112 connecting two adjacent electrode units 410 in the same column have the same length.

Optionally, twenty electrode units 410 are provided with one electrode unit 410 in the first row, four electrode units 410 in each of the middle four rows, and two adjacent electrode units 410 in the last row of three electrode units 410 are spaced apart. The arrangement in rows is distributed in the array area of four rows and six columns, the distance between two adjacent electrode units 410 arranged in rows is the same, and the distance between two adjacent electrode units 410 arranged in columns is different. That is to say, the connecting portions 4112 between two adjacent electrode units 410 connected in a row have the same length. A plurality of connecting portions 4112 connecting two adjacent electrode units 410 in the same row have different lengths.

Optionally, the twenty electrode units 410 are distributed in an arrangement in which four electrode units 410 are arranged in each of the first to fourth columns, three electrode units 410 are arranged in the fifth column, and only one electrode unit 410 is arranged in the last column and in the array area of four rows and six columns. The electrode unit 410 in the last column and one of the three electrode units 410 in the fifth column are aligned in the row direction, and the three electrode units 410 in the fifth column are all arranged adjacent to each other in the row direction. The distance between two adjacent electrode units 410 is the same, the distance between two adjacent electrode units 410 arranged in a row is the same, and the connecting portions 4112 connecting two adjacent electrode units 410 arranged in rows have the same length. A plurality of connection portions 4112 connecting two adjacent electrode units 410 arranged in a row have the same length. Optionally, the electrode units 410 in the last column and the three electrode units 410 in the fifth column are arranged in a row-wise staggered manner, and the three electrode units 410 in the fifth column are all arranged in a row-wise adjacent manner. The spacing between two adjacent electrode units 410 is different, the spacing between adjacent two electrode units 410 arranged in a row is the same, and the connecting portions 4112 connecting the adjacent two electrode units 410 arranged in rows have different lengths. In other words, a plurality of connection portions connecting two adjacent electrode units 410 arranged in a row have the same length. Optionally, the electrode units 10 in the last column and the three electrode units 410 in the fifth column are staggered in the row direction, and among the three electrode units 410 in the fifth column, two adjacent electrode units 410 are arranged in rows at intervals. The spacing between two adjacent electrode units 410 arranged in a row is different, the spacing between two adjacent electrode units 410 arranged in a row is different, and the connecting portions 4112 of a plurality of adjacent two electrode units 410 arranged in a row have different lengths. A plurality of connecting portions 4112 connecting two adjacent electrode units 410 arranged in a row have different lengths.

Optionally, twenty electrode units 410 are arranged in four columns with four electrode units 410 in each column, and at least two of the four columns are distributed in an array area of four rows and six columns at intervals, and two adjacent electrodes arranged in rows. The spacing between the units 410 is different, and the connecting portions 4112 connecting two adjacent electrode units 410 arranged in a row have different lengths. The distance between two adjacent electrode units 410 arranged in a row may be the same or different. A plurality of connection portions 4112 between two adjacent electrode units 410 arranged in a row may have the same length, or may have different lengths.

The twenty electrode units 410 in this embodiment are distributed in an array area of four rows and six columns in such a manner that four electrode units 410 are arranged in each row in the first row and the last row, and six electrode units 410 are arranged in each row in the middle two rows. Inside. From the perspective of column arrangement, each of the first column and the sixth column is provided with two electrode units 410, and each of the middle four columns is provided with four electrode units 410. The electrode units 410 in the same column and in the first column and the sixth column are arranged adjacent to each other in the row direction, and the electrode units 410 in the two columns are arranged in an aligned row direction. Specifically, the four electrode units 410 in the first row are respectively located in the columns from the second column to the fifth column, and the six electrode units 410 in each row of the middle two rows are respectively located in the columns from the first column to the sixth column, the four electrode units 410 in the last row are respectively located in each of the second to fifth columns. The plurality of electrode units 410 of the electrical functional assembly 41 are arranged in an axisymmetric shape. The plurality of electrode units 410 of the electrical functional assembly 41 are arranged axially symmetrically in the row direction and axially symmetrically in the column direction. Twenty electrode units 410 are arranged in an octagonal shape.

The connecting portion 4112 connects all two adjacent electrode units 410 located on the periphery of the array, and at least one of the two adjacent electrode units 410 located on the inner layer of the array is arranged in a disconnected shape. Specifically, the connecting portion 4112 is provided except for the two electrode units 410 located in the third column of the second row and the fourth column of the second row and the two electrode units 410 located in the third column of the third row and the fourth column of the third row. Between all adjacent two electrode units 410 except between. The lengths of the connection portions 4112 connecting two adjacent electrode units 410 arranged in a row are equal. The lengths of the connecting portions 4112 connecting two adjacent electrode units 410 arranged in a row are equal. The connecting portion 4112 is located between two adjacent electrode units 410 arranged in rows, between two electrode units 410 arranged in columns, on the periphery of the array, and located in two adjacent electrode units arranged diagonally in adjacent rows and adjacent columns.

From the distribution position of the electrode units 410 in the array, the plurality of electrode units 410 can be divided into a plurality of peripheral electrode units 410A located at the periphery and a plurality of central electrode units 410B surrounded by the peripheral electrode units 410A. In this embodiment, there are 12 peripheral electrode units 410A, and 8 central electrode units 410B. All the peripheral electrode units 410A are connected in pairs by the connecting portion 4112. That is, the connecting portion 4112 is disposed between all adjacent two peripheral electrode units 410A. At least two of the plurality of center electrode units 410B are arranged in a disconnected state between the center electrode units 410B adjacent in the same row or in the same column, and a space 4C between them is formed for the wiring portion 4113 through.

The gap 4C is set between two adjacent electrode units 410 located in the third column of the second row and the fourth column of the second row and between two adjacent electrode units 410 located in the third column of the third row and the fourth column of the third row between. The connection portion 4113 is located between the electrode units 410 in the third row and the fourth row. The connecting portion 4113 is arranged roughly in a “T” shape, which passes through the gap 4C, and bridges the connecting portion 4112 between the two adjacent electrode units 410 in the middle of the third row and the connection between the two adjacent electrode units 410 in the middle of the fourth row. The connecting portion 4113 and two adjacent connecting portions 4112 connected thereto are arranged in an axisymmetric shape. Optionally, the connecting portion 4113 is arranged in a “˜” shape, and is laterally extended from the connecting portion 4112 corresponding to the gap 4C toward the gap 4C.

The wiring portion 4113 of the electrical function component 41 is electrically connected to the wire 44. In this embodiment, a row of gold fingers 41130 soldered to the wire 44 are provided on both sides of the connecting portion 4113 away from the connecting portion 4112 connected thereto in a staggered shape. One end of the wire 44 is electrically connected to the golden finger 41130 of the wiring portion 4113; the other end is electrically connected to the electric field generator (not shown) through the provided plug 42, so as to provide the insulated electrode 400 with tumor treatment during the tumor electric field treatment. AC signal. A heat-shrinkable sleeve 41 is wrapped around the welding place between the wire 44 and the gold finger 41130 of the connection portion 4113. The heat-shrinkable sleeve 41 insulates and protects the connection between the wire 44 and the wiring portion 4113 of the electrical function component 41, and provides support to prevent the connection between the wire 44 and the connection portion 4113 of the electrical function component 41 from breaking, and at the same time prevent Dust and water resistant.

The electrode unit 410 includes a main body 4111 disposed at opposite ends of the connecting portion 4112, an insulating plate 412 disposed on the side of the main body 4111 away from the human skin, a dielectric element 413 disposed on the side of the main body 4111 facing the human skin, and an optional The temperature sensor 414 is disposed on the main body 4111 and located on the same side as the dielectric element 413. The main body 4111, the insulating plate 412, and the dielectric element 413 are all circular sheet structures. The insulating plate 412, the main body portion 4111 and the dielectric element 413 are disposed in one-to-one correspondence along the thickness direction, and the centers of the three are located on the same straight line. In other embodiments, the main body portion 4111 can also be a cross-point structure extending from the end of the corresponding wiring portion 4112

A conductive disc 4114 is disposed on a side of the main body 4111 facing the dielectric element 413. The conductive plate 4114 of the main body 4111 can be completely covered by the dielectric element 413, so that the conductive plate 4114 and the dielectric element 413 can be welded with solder (not shown). The conductive plate 4114 of the main body 4111 includes a plurality of conductive cores 41140 arranged symmetrically in the center, which can effectively prevent the positional displacement of the dielectric element 413 caused by the accumulation of solder (not shown) during the welding process. The center of the conductive plate 4114 of the main body 4111 is located on the center line of the main body 4111. The top surfaces of the plurality of conductive cores 41140 of the conductive plate 4114 are located on the same plane, which can avoid solder joints between the conductive cores 41140 and the dielectric element 413. The center of the conductive disk 4114 is also located on the center line of the dielectric element 413.

In this embodiment, the conductive plate 4114 of the same main body portion 4111 includes four conductive cores 41140 arranged at intervals and arranged symmetrically in the center. The conductive core 41140 adopts the multi-point interval setting method to reduce the amount of copper foil used to manufacture the conductive core 41140 and reduce material costs. At the same time, it can also save the amount of solder (not shown) used for welding the conductive core 41140 and the dielectric element 413, to further reduce material costs. The four conductive cores 41140 of the same conductive plate 4114 are all petal-shaped. Each conductive core 41140 includes an inner arc (not numbered) and an outer arc (not numbered) connected end to end. The inner arc (not labeled) and the outer arc (not labeled) of the conductive core 41140 are arranged in an axisymmetric shape. The inner arcs (not numbered) of the four conductive cores 41140 of the same conductive plate 4114 are all recessed toward the center of the conductive plate 4114. The outer arcs (not numbered) of the four conductive cores 41140 of the same conductive plate 4114 protrude away from the center of the conductive plate 4114. The four conductive cores 41140 constituting the conductive plate 4114 are arranged in a centrally symmetrical shape and axisymmetrically arranged, and each conductive core 41140 is also arranged in an axially symmetrical shape, so that the four conductive cores 4114 of the main body 4111 conduct electricity. When the core 41140 is welded with the dielectric element 413, the stress balance of each welding point between the conductive plate 4114 and the dielectric element 413 is ensured, the overall welding balance of the dielectric element 413 is ensured, the welding quality is improved, and the welding stress imbalance is avoided to prevent causing the inclination of the electrical element 413 which results in a weak weld at the side where the distance between the dielectric element 413 and the main body 4111 is greater and is easily broken. At the same time, it can avoid affecting the adhesion of the insulated electrode 400.

The insulating plate 412 is made of insulating material. Preferably, the insulating plate 412 is an epoxy glass cloth laminated board. The insulating plate 412 is adhered to the side of the main body 4111 away from the skin of the human body through a sealant (not shown), which can enhance the strength of the main body 4111 and provide a flat welding plane for the welding operation between the main body 4111 and the dielectric element 413, to improve product yield. At the same time, the insulating plate 412 can also isolate the water vapor in the air on the side of the insulating electrode 400 away from the skin from contact with the solder (not shown) between the main body 4111 and the dielectric component 413, to prevent water vapor from corroding the main body 4111 and the dielectric component. The solder (not shown) between 413 affects the electrical connection between the main body 4111 and the dielectric element 413.

The size of the insulating plate 412 is the same as the size of the main body 4111 to prevent the sealant (not shown) from crawling to the side of the main body 4111 away from the human skin through the capillary effect when the insulating plate 412 is pasted on the side of the main body 4111 through the sealant (not shown). The side of the main body 4111 facing the human skin affects the filling of the sealant (not shown) in the gap (not shown) formed by the welding of the dielectric element 413 and the main body 4111, resulting in the presence of the cavities in the sealant (not shown), thereby preventing the sealant (not shown) from curing at high temperatures due to the large difference in thermal expansion coefficient between the water vapor in the cavity and the sealant (not shown), causing the water vapor to expand rapidly, resulting in bursting, popcorning, and damage to the product.

The dielectric element 413 is a material with a high dielectric constant, which has the conductive property of blocking the conduction of direct current and allowing the passage of alternating current, so as to ensure the safety of the human body. Preferably, the dielectric element 413 is a dielectric ceramic sheet. The dielectric element 413 has a ring-shaped structure, and a through hole 4132 is formed in the middle thereof for accommodating the temperature sensor 414. A ring-shaped metal layer (not shown) is attached to the side of the dielectric element 413 facing the main body 4111. A point-to-face welding is formed between the metal layer (not shown) of the dielectric element 413 and the conductive core 41140 of the conductive plate 4114 of the main body 4111, which does not require high welding alignment accuracy, and the welding is more convenient. The gap (not shown) formed by welding the dielectric element 413 and the main body 4111 is filled with a sealant (not shown) to protect the solder (not shown) between the dielectric element 413 and the main body 4111 to protect the solder (not shown) between the dielectric element 413 and the main body 4111 to prevent the dielectric element 413 from being affected by external forces and causing the welding joint to break, thereby causing the alternating electric field to be unable to be applied to the patient's tumor site through the dielectric element 413. At the same time, it can also ensure that the dielectric component 413 is fixed to the main body portion 4111 through a sealant (not shown). The inner ring of the metal layer (not shown) of the dielectric element 413 and the edge of the through hole 4132 of the dielectric element 413 are arranged at intervals, which can avoid the gap between the metal layer (not shown) of the dielectric element 413 and the main body 4111. When the solder (not shown) between them diffuses toward the through hole 4132 of the dielectric element 413 when heated and melted, the temperature sensor 414 is short-circuited. The outer ring of the metal layer (not shown) of the dielectric element 413 and the outer edge of the dielectric element 413 are also arranged at intervals, which can avoid the gap between the metal layer (not shown) and the main body of the dielectric element 413. The solder (not shown) between 4111 overflows to the outside of the main body 4111 when it is heated and melted, so as to prevent the direct current that is not hindered by the dielectric element 413 from passing through and acting on the patient's body when the insulated electrode 400 is attached to the body surface of the tumor site of the patient. surface.

The outer diameter of the dielectric element 413 is slightly smaller than the diameter of the main body 4111. The sealant (not shown) is filled into the gap (not shown) along the edge of the main body 4111 outside the dielectric element 413 through capillarity to facilitate the insertion. The gap (not shown) formed by the welding of the electrical component 413 and the main body 4111 is filled with sealant (not shown). Combined with the through holes 4132 of the dielectric element 413, when the gap (not shown) formed by the welding of the dielectric element 413 and the main body 4111 is filled with sealant (not shown), the air in the gap (not shown) can flow from the gap (not shown). The perforations 4132 of the electrical components 413 are discharged to prevent the sealant (not shown) filled in the gaps (not shown) from causing voids, thereby improving product quality.

There are multiple temperature sensors 414, which are respectively received in the corresponding through holes 4132 of the dielectric element 413. In this embodiment, there are eight temperature sensors 414, which are located in the eight electrode units 410, respectively in the third column of the first row, the fourth column of the first row, the third column of the last row, the fourth column of the last row, the second column of the second row, in the second row and five columns, the third row and second column, and the third row and fifth column. The eight temperature sensors 414 are respectively disposed at the center of the main body portion 4111 of the corresponding electrode unit 410. The temperature sensor 414 is used to monitor the temperature of the adhesive piece 45 covering the side of the dielectric element 413 of the electrical functional component 41 facing the human skin, and further detect the temperature of the human skin attached to the adhesive piece 45. When the temperature monitored by the temperature sensor 414 exceeds the upper limit of the human body's safe temperature, the electric field generator (not shown) promptly reduces or turns off the alternating current to avoid low-temperature burns to the human body. The temperature sensor 414 is welded to the main body 4111 and then sealed with sealant (not shown) to reliably fix the temperature sensor 414 and prevent water vapor from eroding the temperature sensor 414 and causing the temperature sensor 414 to fail. The temperature sensor 414 has a signal terminal (not shown) and a ground terminal (not shown). In other implementations, the specific number of temperature sensors 14 can be set as needed. Temperature sensor 414 is preferably a thermistor.

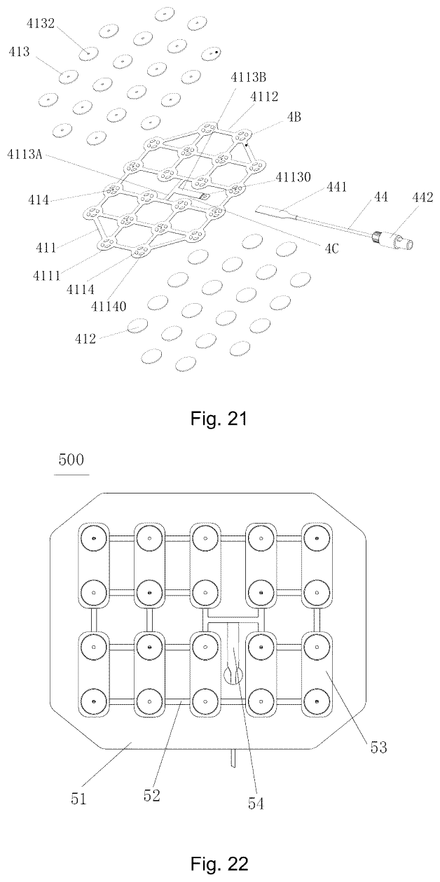

Referring to FIG. 21, the main body portion 4111 of the electrode unit 410 arranged in four rows and six columns, the connecting portion 4112 connecting two adjacent electrode units, and the wiring portion 4113 erected between two adjacent connecting portion 4112 together constitute the flexible circuit board 411 of the electrical function assembly 41. The flexible circuit board 411 is arranged in a grid shape. The dielectric element 413 is disposed on grid points of the flexible circuit board 411. It can be understood that the main body portion 4111 is a grid point of the flexible circuit board 411. From the perspective of the formation of the electrode unit 410, the insulating plate 412 is arranged on the side of the main body 4111 of the flexible circuit board 411 away from the human skin, the dielectric element 413 is arranged on the side of the main body 4111 of the flexible circuit 411 facing the human skin, and the temperature sensor 414 is optionally disposed on the side of the main body 4111 of the flexible circuit board 411 facing the skin of the human body. The arrangement of the main body 4111 of the flexible circuit board 411 is consistent with the arrangement of the electrode units 410.

The flexible circuit board 411 is composed of an insulating substrate 4B and multiple conductive traces (not shown) embedded in the insulating substrate 4B. Conductive traces (not shown) embedded in the insulating substrate 4B of the main body portion 4111, conductive traces (not shown) embedded in the insulating substrate 4B of the connecting portion 4112, and conductive traces (not shown) embedded in the insulating substrate 4B of the connecting portion 4113, the above-mentioned conductive traces (not shown) are electrically connected. The insulating substrate 4B of part of the connecting portion 4112 is embedded with conductive traces (not shown), and the rest of the connecting portion 4112 only includes the insulating substrate 4B to enhance the strength of the flexible circuit board 411. The conductive core 41140 is exposed or protrudes from the insulating substrate 4B of the main body 4111. The insulating substrate 4B of the flexible circuit board 411 can isolate the moisture in the air around the insulating electrode 400 from the solder (not shown) between the conductive core 41140 of the conductive plate 4114 of the main body portion 4111 of the flexible circuit board 411 and the dielectric element 413, to prevent the water vapor in the air on the side away from the skin from eroding the solder (not shown) between the main body 4111 and the dielectric element 413 of the flexible circuit board 411. The insulating substrate 4B of the flexible circuit board 411 and the insulating plate 412 play a double isolation role, which can prolong the service life of the insulating electrode 400. The gold finger 41130 of the connection portion 4113 is exposed on the insulating substrate 4B.

The conductive traces (not shown) of the flexible circuit board 411 include a conductive trace (not shown) that connects all the conductive cores 41140 of the conductive plate 4114 on each main body 4111 in series, and a conductive trace (not shown) that connects all the conductive cores 41140 on the corresponding main body 4111. The ground terminals (not shown) of each temperature sensor 414 are connected in series with conductive traces (not shown) and multiple conductive traces are respectively electrically connected to the signal terminals (not shown) of each temperature sensor 414 on the corresponding main body 4111. The conductive traces (not shown) are electrically connected to the plurality of golden fingers 41130 of the wiring portion 4113 in one-to-one correspondence.

The electrical functional assembly 41 is centrally adhered to the backing 42 through a biocompatible adhesive (not shown), and the backing 42 is provided with a cable hole 421 at the position corresponding to the end of the wiring portion 4113. The cable hole 421 allows one end of the wire 44 to pass through and be electrically connected to the wiring portion 4113, so as to prevent the wire 44 from sticking between the backing 42 and the skin and affect the close contact between the insulating electrode 400 and the skin, and further prevent air from entering between the electrical functional component 41 and human skin, which increases the impedance between the electrical functional component 41 and the skin, thereby causing increased heat generation by the electrical functional component 41 and causing low-temperature burns.

The support member 43 has a plurality of through holes 431 disposed therethrough, and the through holes 431 correspond to the electrode units 410. The supporting member 43 can be a whole sheet structure, which can improve the overall strength of the insulated electrode 400. A plurality of through holes 431 are arranged at intervals and are respectively arranged on the supporting member 43 around the corresponding electrode units 410. In this embodiment, the supporting member 43 is composed of a plurality of independent supporting units 430 with the same structure. A plurality of supporting units 430 are arranged at intervals. Each supporting unit 430 surrounds the periphery of the corresponding plurality of electrode units 410. Each supporting unit 430 has two through holes 431 disposed therethrough, respectively used to accommodate two adjacent electrode units 410 in the same row. The support 43 is composed of ten support units 430. The thickness of the support member 43 is basically the same as that of the electrode unit 410. After the support member 43 and the electrical functional assembly 41 are pasted on the backing 42, the upper surfaces of the support member 43 and the electrode unit 410 are substantially flush. In other embodiment, each supporting unit 430 may be provided with a single through hole 431 with a larger size, surrounding the periphery of the plurality of electrode units 410 in a row.

The sticker 45 is pasted on a side of the support 43 and the electrode unit 410 away from the backing 42. The sticker 45 has double-sided adhesiveness, and can keep the skin surface moist and relieve local pressure when in contact with the skin. The adhesive member 45 is preferably conductive gel. The shape of the sticker 45 is substantially the same as that of the support 43. Because the upper surfaces of the support member 43 and the electrode unit 410 are flush, the adhesive member 45 covers the support member 43 and the electrode unit 410 evenly.

The insulated electrode 400 applies an alternating electric field to the tumor site of the patient through at least 10 electrode units 410 arranged on it for tumor treatment, which can avoid insufficient electric field treatment and affect the treatment effect due to differences in tumor size, location, and position, and increase the size of the insulated electrode. The coverage area of the electrode unit 410 of the insulated electrode 400 is increased, the intensity of the electric field applied to the tumor site for tumor electric field treatment is increased, the range of the alternating electric field covering the tumor site is increased, and the treatment effect is improved.

A plurality of adjacent electrode units 410 are arranged at intervals and form an open space 416 to allow the skin of the corresponding body surface of the patient's tumor site covered by the insulated electrode 400 to be free breathe after the insulated electrode 400 is arranged on the corresponding body surface of the patient's tumor site. The dimensions of the electrode unit 410, the connection portion 412, the connection portion 413 and the size of the open space 416 of the electrical functional assembly 11 will be described in detail below.

The electrode unit 410 is generally in the shape of a circular sheet with a diameter of approximately 21 mm-22 mm. The distance between two adjacent electrode units 410 arranged in a row is the same. The distance between two adjacent electrode units 410 in a row is at least 8 mm. The distance between two adjacent electrode units 410 arranged in a row is the same. The distance between two adjacent electrode units 410 in the same column is at least 6.5 mm. The distance between two adjacent electrode units 410 located in adjacent rows and adjacent columns and arranged diagonally is the same. The distance between two adjacent electrode units 410 arranged diagonally in adjacent rows and adjacent columns is about at least 19 mm.

The electrical functional assembly 41 is generally arranged in an octagonal shape, with a minimum length of 166 mm and a minimum width of 103.5 mm. That is, all the electrode units 410 of the electrical functional assembly 41 are distributed at intervals within a minimum area of 166 mm×10 3.5 mm. This size electrode patch can be used by children with small waist circumference.

In order to make the insulated electrode 400 stick on the corresponding body surface of the patient's tumor site and avoid the overlapping of the electrical functional assembly 41 and affect the treatment effect, the distance between two adjacent electrode units 410 in the same row is at most 22 mm, the maximum distance between two adjacent electrode units 410 located in the same column is 25 mm, and the distance between two adjacent electrode units 410 arranged diagonally in adjacent rows and columns is at least about 33.3 mm. The diameter of the combined electrode unit 410 is at most 22 mm, the length of the electrical functional assembly 41 is at most 242 mm, and the width is at most 166 mm. That is to say, all the electrode units 410 of the electrical functional assembly 41 are distributed at intervals within a maximum area of 242 mm×166 mm. The maximum size of the electrical functional assembly 41 can be suitable for most adult patients. If the patient's waist is too large, two pairs of insulated electrodes 400 can be applied horizontally on the patient's waist for a week. If the waist circumference of the patient is relatively small, two pairs of insulated electrodes 400 can be applied longitudinally on the waist of the patient for one week. If the waist circumference of the patient is appropriate, one pair of insulated electrodes 400 can be applied horizontally to the patient's waist, another pair of insulated electrodes 400 can be applied longitudinally to the patient's waist, and two pairs of insulated electrodes 400 can be applied to the patient's waist for one week.

The length of multiple connecting portions 4112 connecting two adjacent electrode units 410 in the same row is about 8 mm-22 mm, and the length of multiple connecting portions 4112 connecting two adjacent electrode units 410 in the same row is about 6.5 mm-25 mm. The length of the plurality of adjacent two electrode units 410 connected in adjacent rows and adjacent columns and arranged diagonally is about 19 mm-33.3 mm. The width of all connecting portions 4112 between two adjacent electrode units 410 is 4.5 mm-6 mm. Preferably, the width of the connecting portion 4112 is 4.5 mm.

The connection portion 4113 is located between the plurality of electrode units 410 and is disposed in the open space 416 surrounded by the plurality of electrode units 410, so as to prevent the overall size of the electrical functional assembly 41 from being too large, resulting in increased manufacturing costs. The wiring portion 4113 includes a bridging section 4113A bridged between two opposite connecting sections 4112 and a wiring section 4113B connected to the wire 4. The connection section 4113B and the bridging section 4113A are vertically arranged. The bridge section 4113A of the connecting portion 4113 and the two connecting portion 4112 connected thereto are arranged vertically, and the connecting portion 4113B of the connecting portion 4113 is arranged parallel to the two connecting portion 4112 connected to the connecting portion 4113. The bridging section 4113A of the connecting portion 4113 is bridged between two adjacent connecting portions 4112 in the middle, and the connecting section 4113B of the connecting portion 4113 extends laterally from the middle of the bridging section 4113A. The width of the connection segment 4113B of the connecting portion 4113 is at least 4 mm, and the distance between the connection segment 4113B of the connecting portion 4113 and its adjacent electrode unit 410 is at least 2 mm. Preferably, the width of the connection section 4113B of the connecting portion 4113 is 4 mm-8 mm. The width of the bridging section 4113A of the wiring portion 4113 is 4.5 mm-6 mm, and the distance between the bridging section 4113A of the wiring portion 4113 and its adjacent electrode unit 410 is at least 1 mm. Preferably, the width of the bridging section 4113A of the connecting portion 4113 is the same as that of the connecting portion 4112.

Preferably, in this embodiment, referring to FIG. 20 and FIG. 21, the diameter of the electrode unit 410 is 21 mm. The distance between two adjacent electrode units 410 arranged in a row is 15 mm. The distance between two adjacent electrode units 410 arranged in a row is 23 mm. The length of multiple connecting portions 4112 connecting two adjacent electrode units 410 in the same row is 15 mm, the length of multiple connecting portions 4112 connecting adjacent two electrode units 410 in the same row is 23 mm, and a plurality of adjacent electrode units 410 located in adjacent rows and adjacent columns and arranged diagonally have a length of approximately 27.5 mm. The width of the connecting portion 4112 is 4.5 mm. The width of the bridge section 4113A of the connecting portion 4113 is 4.5 mm, the width of the connection section 4113B of the connecting portion 4113 is 8 mm, and the length of the connection section 41138 of the connecting portion 4113 is 42 mm. The electrical functional assembly 41 has a length of 201 mm and a width of 153 mm. The area of the open space 416 formed between the plurality of electrode units 410 where the electrical functional assembly 41 is formed is not completely the same. In this embodiment, among all the open spaces 416, the open space 416 located between the eight electrode units 410 in the middle two rows and through which the wiring portion 4113 passes has the largest area, which is about 4428 mm². The open space 416 located at the four corners of the electrical functional component 41 has the smallest area. The open space 416 located at the corner of the electrical functional component 41 is formed by three electrode units 410 that are adjacent in the same row, adjacent in the same column, and arranged diagonally in adjacent rows and columns, its area is approximately 452 mm². The other open spaces 416 formed by the four electrode units 410 in adjacent rows and adjacent columns have an area of about 1066 mm².

The insulated electrode 400 of this embodiment has an open space 416 located between a plurality of electrode units 410. During the tumor electric field treatment process, the heat accumulated on the skin surface corresponding to the patient's application of the insulated electrode 400 and the water vapor generated by sweating can pass through. The open space 416 is discharged to the outside air, so as to avoid skin discomfort symptoms such as skin erythema, itching, inflammation of hair follicles, pain, and pimples.

### Fifth Embodiment of Insulated Electrodes

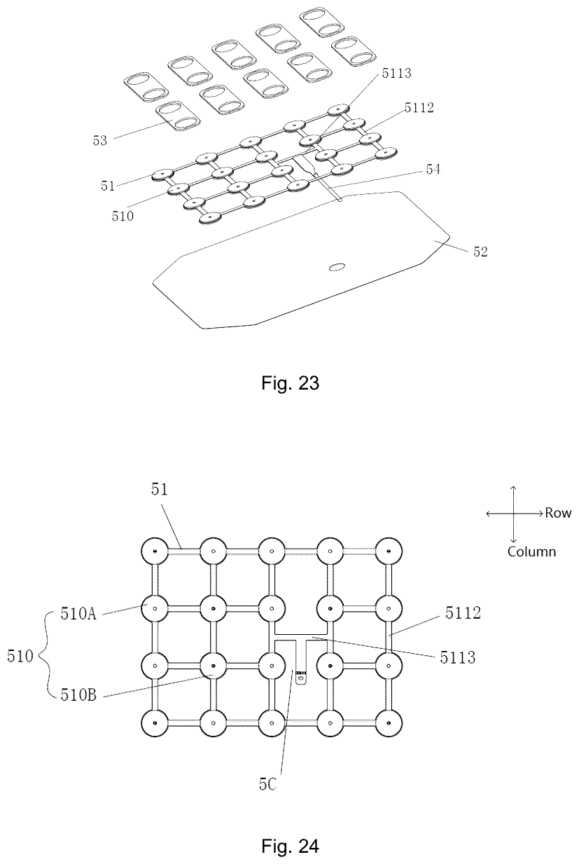

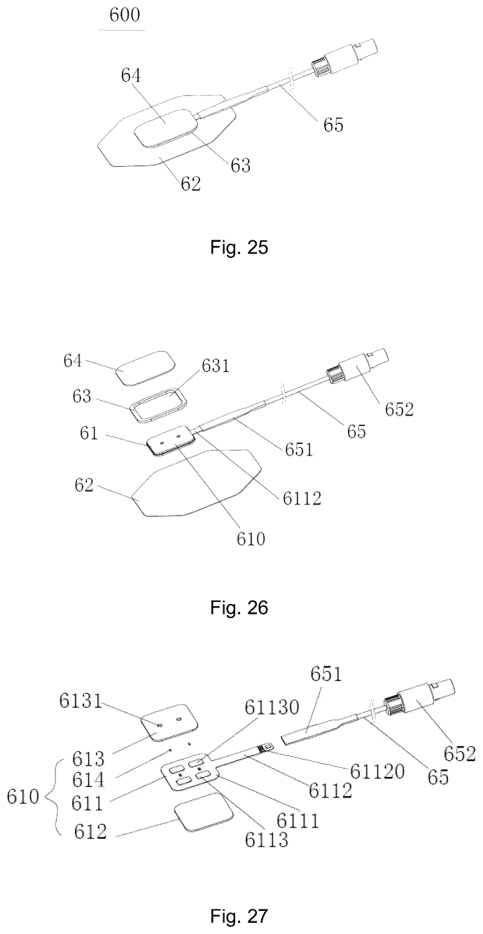

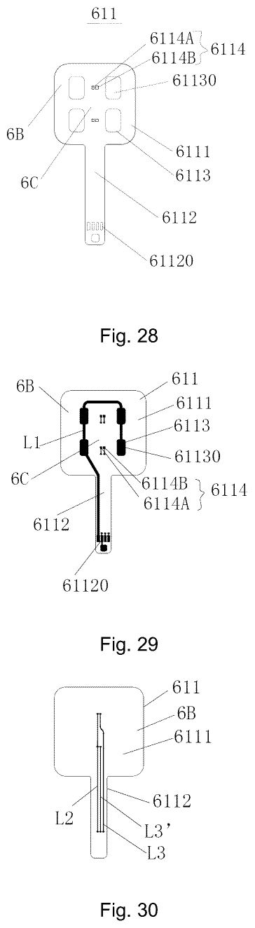

Referring to FIGS. 22 to 24, the insulated electrode 500 of this embodiment includes a backing 52, an electrical functional component 51 adhered to the backing 52, a support 53 adhered to the backing 52, a paste (not shown) covering the support 53 and the corresponding parts of the electrical functional component 51, and a wire 54 electrically connected to the electrical functional component 51.

The insulated electrode 500 in this embodiment is basically the same as the insulated electrode 400 in the fourth embodiment, and the only difference lies in the specific arrangement of the electrode units 510 on the electrical functional component 51. Only the differences will be described below, and for other contents, please refer to the fourth embodiment.

The electrical functional assembly 51 is arranged in a grid shape, and includes a plurality of electrode units 510 arranged in a rectangular array, a plurality of connecting portion 5112 connecting two adjacent electrode units 510, and a wiring portion 5113 electrically connected to the wire 54. Each electrode unit 510 is connected to at least two adjacent electrode units 510 through a connecting portion 5112. Each electrode unit 510 is connected to at least two connecting portion 5112. A plurality of electrode units 510 are distributed on the grid points of the electrical functional assembly 51 at intervals. A plurality of electrode units 510 are distributed in an area surrounded by an array of at least three rows and four columns, and there are at least 12 and at most 30, which can increase the coverage area of the electrode units 510 of the insulated electrode 500 and enhance the application to the tumor. The electric field strength of the tumor electric field treatment can be increased, the range of the alternating electric field covering the tumor site can be increased, and the treatment effect can be improved. A plurality of electrode units 510 are distributed in an array area of three rows and four columns, and the number is 12, or the electrode units 510 are distributed in an array area of three rows and five columns, and the number is at least 12 and at most 15, or the electrode units 510 are distributed in an array area of four rows and four columns, the number is at least 12 and the maximum is 16; or the electrode units 510 are distributed in the array area of four rows and five columns, the number is at least 12 and the maximum is 20, or the electrode units 510 are distributed in the array area of four rows and six columns, the number is at least 12 and the maximum is 24; or the electrode units 510 are distributed in the array area of five rows and five columns, the number is at least 12 and the maximum is 25; or the electrode units 510 are distributed in the array area of five rows and six columns, the number is at least 12 and a maximum of 30.

The number of electrode units 510 in each row is the same and they are aligned in the column direction. The number of electrode units 510 in each column is the same and arranged in a row-aligned manner. The distance between two adjacent electrode units 510 arranged in a row is equal, and the distance between two adjacent electrode units 510 arranged in a column is also equal. Two adjacent electrode units 510 in the same row are arranged in adjacent columns, and two adjacent electrode units 510 in the same column are arranged in adjacent rows. The connecting portion 5112 is located between two adjacent electrode units 510 in the same row or column. A plurality of connection portions 5112 between two adjacent electrode units 510 arranged in a row have the same length. A plurality of connection portions 5112 between two adjacent electrode units 510 arranged in a row have the same length. The distance between two adjacent electrode units 510 arranged in a row is different from the distance between two adjacent electrode units 510 arranged in a column. That is, the length of the connection portion 5112 between two adjacent electrode units 510 arranged in a row is different from the length of the connection portion 5112 between two adjacent electrode units 510 arranged in a row. Optionally, the distance between two adjacent electrode units 510 arranged in a row is the same as the distance between two adjacent electrode units 510 arranged in a column. That is, the length of the connection portion 5112 between two adjacent electrode units 510 arranged in a row is the same as the length of the connection portion 5112 between two adjacent electrode units 510 arranged in a row.

At least one adjacent electrode unit 510 among the plurality of electrode units 510 are arranged in a disconnected shape. A gap 5C is formed between two adjacent electrode units 510 arranged in a disconnected shape, through which the connecting portion 5113 passes. The wiring portion 5113 is arranged in a “˜” shape and extends laterally from a connecting portion 5112 opposite to the interval 5C. The wiring portion 5113 is arranged in a “T” shape and bridges between two connecting portions 5112 respectively connected to the two electrode units 510 arranged in a disconnected manner. The electrode unit 510 arranged in a disconnected shape is located in the inner layer of the array area where the electrode unit 510 is located. The electrode units 510 on the periphery of the electrical functional assembly 51 are all connected in pairs through the connecting portion 5112. That is to say, all two adjacent electrode units 510 located on the periphery of the electrical functional assembly 51 are connected two by two through the connecting portion 5112. At least one of the plurality of electrode units 510 is arranged in a disconnected shape between two adjacent electrode units 510 that are located in adjacent rows and adjacent columns and arranged diagonally. The wiring portion 5113 is located between the plurality of electrode units 510, which can avoid the overall size of the electrical functional assembly 51 from being too large and increase the manufacturing cost.

From the distribution position of the electrode units 510 in the array, the plurality of electrode units 510 can be divided into a plurality of peripheral electrode units 510A located on the periphery and a plurality of central electrode units 510B surrounded by the peripheral electrode units 510A. There are at least 10 peripheral electrode units 510A, and at least two central electrode units 510B. All the peripheral electrode units 510A are connected in pairs by the connecting portion 5112. That is, the connecting portion 5112 is disposed between all adjacent two peripheral electrode units 510A. At least one of the plurality of central electrode units 510B is disconnected from a peripheral electrode unit 510A or a central electrode unit 510B adjacent to the row or column, and a gap 5C is formed between the two, so as to let the wiring portion 5113 pass through.

The wiring portion 5113 may be formed by a connecting portion 5112 opposite to the gap 5C, extending laterally in the direction of the gap 5C, and generally has a “˜” shaped structure. The connecting portion 5112 of the laterally extending connecting portion 5113 is vertically arranged with the connecting portion 5113, and both are arranged in a substantially “T” shape. The connecting portion 5112 of the laterally extending wiring portion 5113 is located between two adjacent peripheral electrode units 510A. It can also be located between one peripheral electrode unit 510A and an adjacent center electrode unit 510B. It can also be located between two adjacent peripheral electrode units 510A. That is, the connecting portion 5112 of the laterally extending wiring portion 5113 connects two adjacent peripheral electrode units 510A, or connects two adjacent central electrode units 510B, or connects a peripheral electrode unit 510A and its adjacent central electrode unit 510B. The wiring portion 5113 can also be arranged in a “T” shape, erected between the two connecting portions 5112 respectively connected to the two central electrode units 510B arranged in a disconnected shape, or erected with a central electrode arranged in a disconnected shape. The unit 510B and a peripheral electrode unit 510A adjacent to the center electrode unit 510B are respectively connected between two connecting portions 5112.

In other embodiment, among the plurality of peripheral electrode units 510A, at least one adjacent two peripheral electrode units 510A are arranged in a disconnected manner, and at least one of the peripheral electrode units 510A arranged in the disconnected manner is connected with one central electrode units 510B diagonally arranged in adjacent row and in adjacent column thereto through the connecting portion 5112. That is, some of the adjacent two peripheral electrode units 510A are connected with each other through the connecting portion 5112; some of the adjacent two peripheral electrode units 510A are arranged in a disconnected manner and no connecting portion 5112 is located therebetween; the connecting portion 5112 is located between a peripheral electrode unit 510A and a central electrode unit 510B adjacent to the peripheral electrode unit 510A and arranged diagonally in adjacent row and adjacent column, or between two adjacent peripheral electrode units 510A, or between two adjacent central electrode units 510B, or between a peripheral electrode unit 510A and a center electrode units 510B which is adjacent to the peripheral electrode unit 510A and also located in the same row or in the same column with the peripheral electrode unit 510A.

In this embodiment, the plurality of electrode units 510 of the electrical functional assembly 51 are arranged in four rows and five columns. The number of electrode units 510 of the electrical functional assembly 51 is twenty. The number of electrode units 510 in each row is the same and the number of electrode units 510 in each column is also the same. The number of electrode units 510 in each row is five. The number of electrode units 510 in each row is four. The electrode unit 510 located in the second row, third column and the electrode unit 510 located in the second row, fourth column are arranged in a disconnected shape, and a gap 5C is formed therebetween. The electrode unit 510 located in the third row and the third column and the electrode unit 510 located in the third row and the fourth column are arranged in a disconnected shape, and a gap 5C is also formed between them. The connecting portion 5113 is arranged in a “T” shape, and bridged between the connecting portion 5112 located in the middle of the third column and the connecting portion 5112 located in the middle of the fourth column. The connecting portion 5112 in the middle of the third column is disposed between the two electrode units 510 located in the second row of the third column and the fourth row of the third column. The connecting portion 5112 in the middle of the fourth column is disposed between the two electrode units 510 located in the second row of the fourth column and the third row of the fourth column. the connecting portion 5112 is located in all the two-electrode units 510 in the same row or in the same column except the two-electrode units 510 in the second row, third column and second row, fourth column and the two-electrode units 510 in the third row, third column, and third row, fourth column. between two adjacent electrode units 510.

The insulated electrode 500 of this embodiment applies an alternating electric field to the tumor site of the patient through the plurality of electrode units 510 provided thereon for tumor treatment, which can avoid the occurrence of electric field therapy applied to the tumor site due to differences in tumor size, location, and position. Insufficient alternating electric field intensity affects the therapeutic effect, increase the coverage area of the electrode unit 510 of the insulated electrode 500, increase the electric field intensity applied to the tumor site for tumor electric field therapy, increase the range of the alternating electric field covering the tumor site, and improve the therapeutic effect.

### Sixth Embodiment of Insulated Electrodes

The electrical functional assembly of the insulated electrodes in the previous embodiments are provided with a plurality of electrode units, which are arranged in series. If one of the electrode units is damaged, the entire insulated electrode will be scrapped, and the scrapping cost is relatively high. Therefore, this embodiment provides another form of insulated electrodes. 25 to 30 show the insulating electrode 600 of the sixth embodiment. Multiple insulated electrodes 600 in this embodiment can be used in combination, and multiple insulated electrodes 600 are connected to a hub (not shown) to jointly perform tumor electric field therapy on tumor sites. The insulated electrode 600 includes a backing 62, an electrical functional assembly 61 adhered to the backing 62, a support 63 adhered to the backing 62, covering the corresponding parts of the support 63 and the electrical functional assembly 61, and is connected to the tumor site of the patient. Corresponding stickers 64 attached to the body surface and wires 65 electrically connected to the electrical functional assembly 61. The insulated electrode 600 is attached to the body surface corresponding to the patient's tumor site through the backing 62, and an alternating electric field is applied to the patient's tumor site through the electrical functional assembly 61 to interfere or prevent the mitosis of the patient's cancer cells, thereby achieving the purpose of treating the tumor.

The electrical functional assembly 61 includes a single electrode unit 610 arranged in the shape of a square sheet and a wiring portion 6112 connected to the electrode unit 610. The wiring portion 6112 is welded to the wire 65 to realize the electrical connection between the electrical functional assembly 61 and the wire 65. A plurality of golden fingers 61120 are provided on one side surface of the connection portion 6112. In this embodiment, there are four gold fingers 61120, and the four gold fingers 61120 are arranged on the surface of the connecting portion 6112 facing the skin. A heat-shrinkable sleeve 651 is wrapped around the welding place between the wire 65 and the gold finger 61120 of the connection portion 6112. The heat-shrinkable sleeve 651 insulates and protects the connection between the wire 65 and the wiring portion 6112 of the electrical function component 61, and provides support to prevent the connection between the wire 65 and the connecting portion 6112 of the electrical function component 61 from breaking, and at the same time prevent Dust and water resistant. A plug 652 electrically connected to an electric field generator (not shown) or a hub (not shown) is provided at the end of the wire 65 away from the wiring portion 6112. One end of the wire 65 is electrically connected to the gold finger 61120 of the wiring portion 6112; the other end is electrically connected to the electric field generator (not shown) or the hub (not shown) through the plug 652, so as to serve as an insulated electrode 600 during the electric field treatment of the tumor. Provides alternating current signals for tumor therapy.

The electrode unit 610 includes a main body 6111, an insulating plate 612 disposed on the side of the main body 6111 away from human skin, a dielectric element 613 disposed on the side of the main body 6111 facing the human skin, and a dielectric element 613 disposed on the main body 6111 and connected to the dielectric element. 613 are two temperature sensors 614 located on the same side. The main body 6111, the insulating plate 612, and the dielectric element 613 are roughly the same in shape, and are all square sheet structures. The main body 6111, the insulating plate 612, and the dielectric element 613 are arranged correspondingly along the thickness direction of the main body 6111, and the centers of the three are located on the same straight line. In this embodiment, the main body 6111, the insulating plate 612 and the dielectric element 613 are all square sheet-shaped structures with arc-shaped corners. Preferably, the main body portion 6111 is in the shape of a square plate with a size of about 32 mm×32 mm. The wiring portion 6112 of the electrical functional assembly 61 is laterally extended from the main body portion 6111 of the electrode unit 610.

The main body 6111 is composed of an insulating substrate 6B and four conductive traces L embedded in the insulating substrate 6B. The four conductive traces are respectively a first conductive trace L1 arranged on the side of the insulating substrate 6B close to the dielectric element 613, a second conductive trace L2 arranged on the side of the insulating substrate 6B close to the insulating plate 612, and two A third conductive trace L3, L3′ on the same side as the second conductive trace L2. A conductive pad 6113 is centrally disposed on the main body portion 6111 to expose the insulating substrate 6B and to be electrically connected to the first conductive trace L1. The conductive plate 6113 can be welded with the dielectric element 613 to assemble the dielectric element 613 on the main body 6111. The conductive plate 6113 can be completely covered by the dielectric element 613, so that the conductive plate 6113 and the dielectric element 613 can be welded with solder (not shown). The center of the conductive plate 6113 is located on the centerline of the main body 6111. The conductive plate 6113 includes a plurality of conductive cores 61130 symmetrically arranged in the center, which can effectively prevent the positional displacement of the dielectric element 613 due to the accumulation of solder (not shown) during the welding process. The top surfaces of the plurality of conductive cores 61130 are located on the same plane, which can avoid false welding with the dielectric element 613 during welding. The plurality of conductive cores 61130 are all connected to the first conductive trace L1. A plurality of conductive cores 61130 are connected in series by the first conductive trace L1.

In this embodiment, the conductive plate 6113 of the main body portion 6111 is roughly square in shape, and its symmetry axis coincides with the symmetry axis of the main body cloth 111. The conductive plate 6113 includes four conductive cores 61130 located at four corners and arranged at intervals. Conductive core 61130 adopts multi-point interval setting method to reduce the amount of copper foil used to manufacture conductive core 61130; at the same time, it can also save the amount of solder (not shown) used to weld conductive core 61130 and dielectric element 613, reducing manufacturing costs. Each conductive core 61130 has a rectangular configuration with dimensions of approximately 9 mm×6 mm. Preferably, each conductive core 61130 is in the shape of a rectangle with rounded corners. The longitudinal axis of each conductive core 61130 is parallel to the extending direction of the connecting portion 6112. In other implementation manners, each conductive core 61130 of the conductive plate 6113 can also be circular, square, etc.

In this embodiment, the four conductive cores 61130 constituting the conductive plate 6113 are arranged in a matrix, and the four conductive cores 61130 are arranged in two rows and two columns. The gap between two columns of conductive cores 61130 is about 8.5 mm, and the gap between two rows of conductive cores 61130 is about 4 mm. The four conductive cores 61130 constituting the conductive disc 6113 are arranged in a centrally symmetrical shape and axisymmetrically arranged, and each conductive core 61130 is also arranged in an axisymmetrically shaped shape, so that the four conductive cores 61130 of the main body 6111 and the intermediate When the electric element 613 is welded, the stress of each welding point is balanced to ensure the overall welding balance of the dielectric element 613, improve the welding quality, and avoid the gap between the dielectric element 613 and the main body 6111 caused by the inclination of the dielectric element 613 due to unbalanced welding stress. The strength of the weld on the large side is weak and easy to break; at the same time, it can avoid affecting the fit of the insulated electrode 600. The four conductive cores 61130 of the conductive plate 6113 are arranged in two-to-two intervals, and a gap 6C is formed between two adjacent conductive cores 61130. The four compartments 6C are provided approximately in the shape of a “ten” connected to each other. Adjacent intervals 6C are provided in a continuous state. Two of the four spaces 6C located between the two conductive cores 61130 in the same row extend in the same direction as the connecting portion 6112.

The main body 6111 is also provided with two pairs of pads 6114 exposing its insulating substrate 6B, which can be soldered to the corresponding parts of the corresponding temperature sensor 614 to realize the electrical connection between the temperature sensor 614 and the main body 6111. Each pair of pads 6114 is located between two conductive cores 61130 that are spaced apart in a corresponding row. The two pairs of pads 6114 are located in the extending direction of the connection portion 6112, and each pair of pads 6114 has a center of symmetry, and the line connecting the two centers of symmetry of the two pairs of pads 6114 is parallel to the extending direction of the connection portion 6112. Each pair of pads 6114 includes a first pad 6114A and a second pad 6114B. The first pad 6114A of each pair of pads 6114 is electrically connected to the second conductive trace L2, one of the two second pads 6114B is electrically connected to the third conductive trace L3, and the other is electrically connected to the third conductive trace L3. The trace L3′ is electrically connected. Each temperature sensor 614 has a signal terminal (not shown) and a ground terminal (not shown). The first pad 6114A is soldered to the ground terminal (not shown) of the temperature sensor 614, and the second pad 6114B is soldered to the corresponding signal terminal (not shown) of the temperature sensor 614.

The insulating plate 612 is made of insulating material. Preferably, the insulating board 612 is an epoxy glass cloth laminated board. The insulating plate 612 is adhered to the side of the main body 6111 away from human skin by a sealant (not shown), which can enhance the strength of the main body 6111 and provide a flat welding plane for the welding operation between the main body 6111 and the dielectric element 613, Improve product yield. At the same time, the insulating plate 612 can also isolate the water vapor in the air on the side away from the skin of the insulating electrode 600 from contacting the solder (not shown) between the main body 6111 and the dielectric element 613 to prevent water vapor from corroding the main body 6111 and the dielectric element The solder (not shown) between 613 affects the electrical connection between the main body 6111 and the dielectric element 613.

The size of the insulating plate 612 is the same as that of the main body portion 6111, so as to avoid that when the insulating plate 612 is pasted on the side of the main body portion 6111 away from the skin of the human body through a sealant (not shown), the sealant (not shown) will crawl to The main body 6111 faces the side of the human skin, which affects the filling of the sealant (not shown) in the gap (not shown) formed by welding the dielectric element 613 and the main body 6111, resulting in the presence of sealant (not shown) Cavities, thereby preventing the sealant (not shown) from exploding rapidly due to the large difference in thermal expansion coefficient between the water vapor in the cavity and the sealant (not shown) when the sealant (not shown) is cured at high temperature, resulting in bursting, popcorn phenomenon, and damage to the product.

The dielectric element 613 is made of a material with a high dielectric constant, which has a conductive property of blocking the conduction of direct current and allowing the passage of alternating current, which can ensure the safety of the human body. Preferably, the dielectric element 613 is a dielectric ceramic sheet. The dielectric element 613 is penetrated with two through holes 6131 whose number is the same as that of the temperature sensors 614, respectively for accommodating corresponding temperature sensors 614. A metal layer (not shown) is attached to the side of the dielectric element 613 facing the main body 6111. A point-to-face welding is formed between the metal layer (not shown) of the dielectric element 613 and the conductive core 61130 of the conductive plate 6113 of the main body 6111, which does not require high welding alignment accuracy, and the welding is more convenient. The inner edge of the metal layer (not shown) of the dielectric element 613 and the edge of the through hole 6131 of the dielectric element 613 are arranged at intervals, which can avoid the gap between the metal layer (not shown) of the dielectric element 613 and the main body 6111. When the solder (not shown) between them diffuses toward the through hole 6131 of the dielectric element 613 when heated and melted, the temperature sensor 614 is short-circuited. The outer edge of the metal layer (not shown) of the dielectric element 613 and the outer edge of the dielectric element 613 are also arranged at intervals, which can avoid the gap between the metal layer (not shown) and the main body of the dielectric element 613. When the solder (not shown) between 6111 is heated and melted, it overflows to the outside of the main body 6111, so that when the insulated electrode 600 is attached to the body surface of the patient's tumor site, the direct current that is not hindered by the dielectric element 613 passes through and acts on the patient's body. surface.

The gap (not shown) formed by welding the dielectric element 613 and the main body 6111 is filled with sealant (not shown) to protect the solder (not shown) between the dielectric element 613 and the main body 6111 to avoid dielectric The component 613 is affected by the external force and causes the welding place to break, and then the alternating electric field cannot be applied to the tumor site of the patient through the dielectric component 613; at the same time, it can also prevent the water vapor in the air from entering the gap (not shown) to corrode the dielectric component 613 in the solder (not shown) between the main parts 6111 further affects the electrical connection between the dielectric element 613 and the main part 111. The size of the dielectric element 613 is slightly smaller than the size of the main body 6111, so that the sealant (not shown) can pass through the capillary phenomenon along the edge of the main body 6111 outside the dielectric element 613 when filling the sealant (not shown). Filling into the gap (not shown) facilitates the filling of the sealant (not shown) in the gap (not shown) formed by welding the dielectric element 613 and the main body 6111. When the gap (not shown) formed by welding the dielectric element 613 and the main body 6111 is filled with sealant (not shown), the air in the gap (not shown) can be discharged from the perforation 6131 of the dielectric element 613 to avoid The sealant (not shown) filled in the gap (not shown) creates voids to improve product quality.

Each temperature sensor 614 is welded with the first pad 6114A provided on the main body 6111 through its ground terminal (not shown) and its signal terminal (not shown) is welded with the second pad 6114B provided on the main body 6111 to Realize the electrical connection between it and the main body portion 6111. Since the two first pads 6114A of the main body 6111 are electrically connected to the second conductive trace L2, one of the two second pads 6114B is electrically connected to the third conductive trace L3, and the two second pads 6114B are electrically connected to the third conductive trace L3. The other of the pads 6114B is electrically connected to the third conductive trace L3′, while the two first pads 6114A are soldered to the corresponding ground terminals (not shown) of the two temperature sensors 614 respectively, and the two second pads 6114B Solder with corresponding signal terminals (not shown) of the two temperature sensors 614 respectively, thus, the ground terminals (not shown) of the two temperature sensors 614 are electrically connected with the second conductive trace L2 of the main body 6111, Signal terminals (not shown) of the two temperature sensors 614 are electrically connected to the third conductive traces L3, L3′ of the main body 6111. That is, the two temperature sensors 614 transmit their monitored temperature signals through the second conductive trace L2 and the third conductive trace L3, L3′. The two temperature sensors 614 are respectively accommodated in the corresponding through holes 6131 of the dielectric element 613 after being welded on the main body 6111. Preferably, the temperature sensor 614 is a thermistor. The temperature sensor 614 is used to monitor the temperature of the sticker 64 covering the side of the dielectric element 613 of the electrical functional assembly 61 facing the human skin, and further detect the temperature of the human skin attached to the sticker 64. When the temperature detected by the temperature sensor 614 exceeds the upper limit of the safe temperature of the human body, the tumor electric field therapy system (not shown) can promptly reduce or turn off the alternating voltage applied to the insulating electrode 600 to avoid low-temperature burns on the human body. The two temperature sensors 614 are arranged symmetrically on the main body 6111, which can detect the temperature of human skin corresponding to different positions and ensure the reliability of the detected data. The two temperature sensors 614 are welded to the main body 6111 through the two pairs of pads 6114 of the main body 6111 and then sealed with a sealant (not shown) to prevent water vapor from corroding the temperature sensors 614 and causing the temperature sensors 614 to fail.

the connecting portion 6112 has the same structure as the main body portion 6111, and also has a corresponding insulating substrate 6B and four conductive traces L embedded in the insulating substrate 6B. The four conductive traces L of the connecting portion 6112 are also electrically connected to the corresponding conductive traces L of the main body 6111. The four golden fingers 61120 of the connection portion 6112 are all exposed from a side of the insulating substrate 6B that is close to the dielectric element 613. The four conductive traces L of the connecting portion 6112 are respectively electrically connected to the golden fingers 61120. The four conductive traces L of the connecting portion 6112 are also respectively the first conductive trace L1, the second conductive trace L2 and the third conductive traces L3, L3′. The first conductive trace L1 of the connecting portion 6112 is extended from the first conductive trace L1 of the main body portion 6111. The second conductive trace L2 of the connecting portion 6112 is extended from the second conductive trace L2 of the main body portion 6111. The third conductive traces L3, L3′ of the connecting portion 113 are respectively extended from the corresponding third conductive traces L3, L3′ of the main body portion 6111.

the connecting portion 6112 is connected to the first conductive trace L1 of the main body 6111 through its first conductive trace L1, and the first conductive trace L1 of the main body 6111 is connected to the conductive plate 6113 on the main body 6111 to realize the connection with the main body 6111. The electrical connection between the conductive pads 6113, and the electrical connection between the conductive pads 6113 of the main body 6111 and the dielectric component 613 are realized by welding the dielectric component 613. the connecting portion 6112 is connected to the second conductive trace L2 of the main body portion 6111 through its second conductive trace L2, and the connection between the second conductive trace L2 of the main body portion 6111 and the first pad 6114A on the main body portion 6111 realizes the connection with the main body The electrical connection of the first pad 6114A on the part 6111, and then realize the connection between the first pad 6114A and the ground terminal (not shown) of the temperature sensor 614 by welding the first pad 6114A and the ground terminal (not shown) of the temperature sensor 614 electrical connection, the connecting portion 6112 is respectively connected to the third conductive traces L3, L3′ of the main body 6111 through its third conductive traces L3, L3′, and the third conductive traces L3, L3′ of the main body 6111 are respectively connected to the two second conductive traces. The connection of the two pads 6114B realizes the electrical connection with the two second pads 6114B on the main body 6111, and then connects the signal terminals (not shown) of the two temperature sensors 614 through the two second pads 6114B. A corresponding welding realizes its electrical connection with the signal terminals (not shown) of the two temperature sensors 614, so that the temperature signals monitored by the temperature sensors 614 are transmitted to the electric field generator (not shown) in parallel, so that the electric field The generator (not shown) can timely and efficiently adjust the alternating voltage or alternating current applied to the dielectric element 613 to avoid low-temperature burns caused by excessive temperature.

The main body portion 6111 and the connecting portion 6112 jointly constitute the flexible circuit board 611 of the electrical function component 61. The insulating substrate 6B of the main body portion 6111 and the connection portion 6112 together constitute the insulating substrate 6B of the flexible circuit board 611. The conductive traces L of the main body portion 6111 correspond to the conductive traces L of the connection portion 6112 to constitute the conductive traces L of the flexible circuit board 611. The insulating substrate 6B of the flexible circuit board 611 can isolate the water vapor in the air around the insulating electrode 600 and the solder (not shown) between the conductive plate 6113 and the dielectric element 613, avoiding the water vapor in the air on the side away from the skin from eroding the device. Solder (not shown) between the conductive plate 6113 on the main body portion 6111 of the flexible circuit board 611 and the flexible circuit board 611 and the dielectric element 613. The insulating substrate 6B of the flexible circuit board 611 and the insulating plate 612 play a double isolation role, which can prolong the service life of the insulating electrode 600.

From the perspective of the formation of the electrode unit 610, the insulating plate 612 is arranged on the side of the main body 6111 of the flexible circuit board 611 away from the human skin, and the dielectric element 613 is arranged on the side of the main body 6111 of the flexible circuit board 611 facing the human skin. The temperature sensor 614 is disposed on the side of the main body 6111 of the flexible circuit board 611 facing the skin of the human body. The insulating board 612 and the dielectric element 613 are respectively disposed on opposite sides of the main body portion 6111 of the flexible circuit board 611. The first conductive trace L1 of the flexible circuit board 611 connects the four spaced conductive cores 61130 of the conductive plate 6113 in series, and the second conductive trace L2 connects the ground terminals of the two temperature sensors 614 through the two first pads 6114A (not shown) are electrically connected, and the third conductive traces L3, L3′ are respectively electrically connected to the signal terminals (not shown) of the two temperature sensors 614 through the two second pads 6114B. The first conductive trace L1 is located in a layer of the insulating substrate 6B close to human skin. Both the second conductive trace L2 and the third conductive traces L3, L3′ are located on a layer close to the insulating board 612 in the insulating substrate 6B. In order to facilitate the laying of the conductive traces L, the width of the connection portion 6112 is 7-9 mm. Preferably, the width of the connecting portion 6112 is 8 mm.

The gold finger 61120 of the connecting portion 6112, the four conductive cores 61130 of the conductive plate 6113 and the pad 6114 all expose a side of the insulating substrate 6B of the flexible circuit board 611 that is close to the dielectric element 613. The gold finger 61120, the four conductive cores 61130 of the conductive plate 6113 and the pad 6114 are all located on the side of the flexible circuit board 611 close to the patient's body surface. One end of a gold finger 61120 of the wiring portion 6112 is electrically connected to the dielectric element 613 through the first conductive trace L1 connected thereto, and the other end is welded to the corresponding part of the wire 65 to connect the electric field generator (not shown) The alternating voltage signal is transmitted to the dielectric element 613. One end of one gold finger 61120 of the other three gold fingers 61120 of the wiring portion 6112 is electrically connected to the ground terminal (not shown) of the temperature sensor 614 through the second conductive trace L2 connected thereto, and one end of the other two gold fingers 61120 is electrically connected to the ground terminal (not shown) through the second conductive trace L2 connected thereto. The third conductive traces L3, L3′ are respectively electrically connected to signal terminals (not shown) of the two temperature sensors 614. The other ends of the three gold fingers 61120 of the wiring portion 6112 are respectively welded to the corresponding parts of the wire 65, so as to realize the relevant signal detected by the temperature sensor 614 through the second conductive trace L2, the third conductive trace L3, L3′, Wires 65 run in parallel to an electric field generator (not shown).

The backing 62 is arranged in sheet form, which is mainly made of flexible and breathable insulating material. The backing 62 is a mesh fabric. Specifically, the backing 62 is a mesh non-woven fabric, which is soft, light, moisture-proof, and breathable, and can keep the patient's skin surface dry after long-term sticking on the patient's body surface. The side of the backing 62 facing the patient's body surface is also coated with a biocompatible adhesive (not shown), which is used to closely adhere the backing 62 to the corresponding body surface of the patient's tumor site. In this embodiment, the backing 62 is arranged in an approximately octagonal sheet shape.

The supporting member 63 is adhered to the backing 62 and surrounds the outer side of the electrode unit 610. A through hole 631 is formed in the middle of the support member 63 for accommodating the electrode unit 610. The supporting member 63 may be made of foam material. The support member 63 is flush with the surface of the electrode unit 610 away from the backing 62. That is, the supporting member 63 is flush with the surface of the electrode unit 610 facing the sticker 64 to support the sticker 64.

The sticker 64 has double-sided adhesive. One side of the adhesive member 64 is glued on the support member 63 and the surface of the electrode unit 610 away from the backing 62. The other side of the sticker 64 is used as an application layer, which is applied on the surface skin of the human body to keep the skin surface moist and relieve local pressure. Preferably, the sticker 64 is a conductive hydrogel to serve as a conductive medium. Under the support of the supporting member 63, the sticker 64 has better adhesion to human skin.

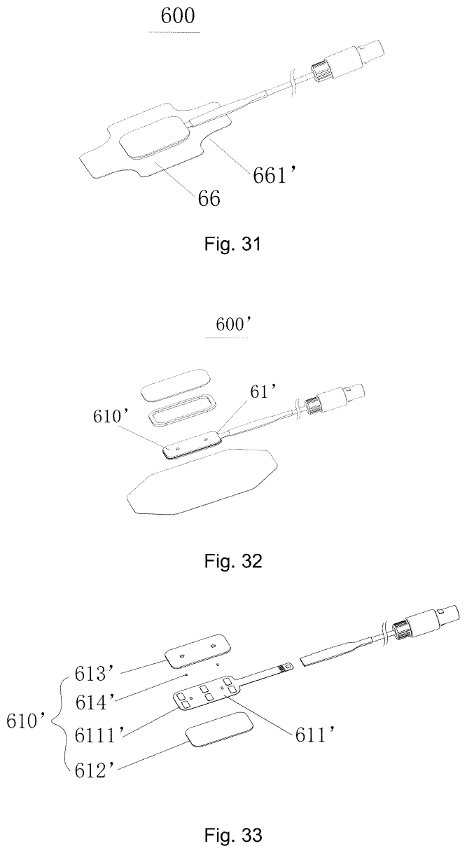

FIG. 31 shows a backing 66 of another embodiment of the backing of the insulated electrode 600. The four corners of the backing 66 are recessed inwardly with concave corners 661. The backing 66 is generally in a “cross” configuration. The concave corner 661 communicates with the outside and is arranged in an “L” shape. When the insulated electrode 600 is attached to the body surface corresponding to the patient's tumor site, the concave corner 661 can prevent the corners of the backing 66 from arching and causing wrinkles, thereby preventing air from entering the gap between the electrode unit 610 and the skin from the folds to increase the contact between the electrical function component 61 and the skin. The impedance between the electrical functional assembly 61 increases heat generation and causes low-temperature burns.

Since the insulated electrode 600 in this embodiment uses a separate electrode unit 610 to apply an alternating voltage to the patient's tumor site, when it fails to work normally, only the insulated electrode 600 with a separate electrode unit 610 needs to be replaced, and there is no need to replace the insulated electrode 600 with multiple electrode units. The entire insulated electrode 600 of each electrode unit 610 is scrapped, which can reduce the cost of tumor treatment for patients. In addition, the insulated electrodes 600 of this embodiment can be freely combined in number according to the patient's tumor site and the size of the patient's tumor site, so as to ensure the coverage area of the insulated electrode 600 for tumor electric field therapy and ensure the intensity of the electric field therapy; and the multiple insulated electrodes 600 The relative position between them can also be freely adjusted according to the patient's own physical differences, tumor location, and tumor size, so as to obtain the optimal electric field intensity and electric field coverage area for tumor treatment, and at the same time allow the skin energy of the patient's body surface with the insulated electrode 600 Breathe freely, avoiding the accumulation of heat on the patient's body surface due to long-term tumor electric field treatment, which cannot be dissipated in time, causing sweating to block pores and produce skin inflammation.

In addition, the flexible circuit board 611 of the insulated electrode 600 of this embodiment is only provided with a first conductive trace L1 electrically connected to the dielectric element 613, which is common to the ground terminals (not shown) of the two temperature sensors 614. One second conductive trace L2 electrically connected and two third conductive traces L3, L3′ respectively electrically connected to the signal ends (not shown) of the two temperature sensors 614, so as to realize the electric field generator The alternating voltage signal (not shown) is transmitted to the dielectric element 613 through the first conductive trace L1 to achieve the purpose of applying an alternating voltage to the tumor site of the patient for tumor treatment; at the same time, it communicates with the first conductive trace L2 through the second conductive trace L2 The three conductive traces L3, L3′ are respectively electrically connected to the two temperature sensors 614 to realize the signal transmission between the electric field generator (not shown) and the two temperature sensors 614, the wiring design is less difficult, the structure is simple, and the manufacturing process is simplified, manufacture is easy, and the product manufacturing yield rate is high, can greatly reduce manufacturing cost low.

### Alternative Implementation of the Sixth Embodiment of Insulated Electrodes

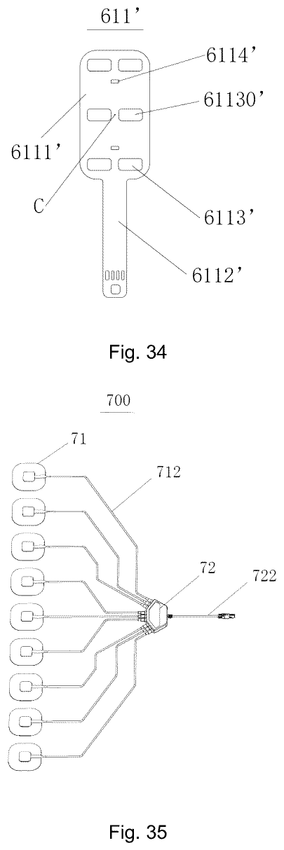

FIG. 32 to FIG. 34 show the insulated electrode 600′ of the transformation implementation of the sixth embodiment, the structure of the insulated electrode 600′ is basically the same as that of the insulated electrode 600, the main difference is: the electrode of the electrical function component 61′ of the insulated electrode 600′ The shape of the unit 610′ is rectangular, and the electrode unit 610′ includes a main body 6111′ of a flexible circuit board 611′, an insulating plate 612′, a dielectric element 613′, and is arranged on the main body 6111′ and is located with the dielectric element 613′. Two temperature sensors 614′ on the same side. The main body portion 6111′, the insulating plate 612′ and the dielectric element 613′ are all rectangular sheet structures with rounded corners. Preferably, the main body portion 6111′ is in the shape of a rectangular sheet with a size of about 43.5 mm×23.5 mm. The arrangement of the conductive plate 6113′ and the two pairs of pads 6114′ on the surface of the main body 6111′ is also slightly different, which will be described in detail below, wherein the flexible circuit board 61 also includes a wire (not labeled) extending from the main body 6111′ and connected to it. The wiring portion 6112′ of the flexible circuit board 611′ is the same as that of the flexible circuit board 611 in the sixth embodiment, and will not be repeated here.

The conductive plate 6113′ of the main body 6111′ includes six conductive cores 61130′ located at its four corners and in the middle of its two long sides and arranged at intervals. Each conductive core 61130′ has a rectangular configuration with dimensions of approximately 8 mm×4 mm. Preferably, each conductive core 61130′ is in the shape of a rectangle with rounded corners. The six conductive cores 61130′ constituting the conductive plate 6113′ are arranged at intervals in a matrix, and the six conductive cores 61130′ are arranged in three rows and two columns along the longitudinal direction of the main body 6111′. There are 2 conductive cores 61130′ in the first row, 2 conductive cores 61130′ in the middle row, and 2 conductive cores 61130′ in the last row. The gap between two rows of conductive cores 61130′ is about 2.4 mm, and the gap between conductive cores 61130′ in adjacent rows is about 12.8 mm. The six conductive cores 61130′ constituting the conductive plate 6113′ are arranged in a centrally symmetrical shape and axisymmetrically arranged, and each conductive core 61130′ is also arranged in an axisymmetrically shaped shape, so that the six conductive cores of the main body 6111′ When the core 61130′ is welded with the dielectric element 613′, the stress of each welding point is balanced, ensuring the overall welding balance of the dielectric element 613′, improving the welding quality, and avoiding the inclination of the dielectric element 613′ due to unbalanced welding stress. The welding part on the side where the distance between the element 613′ and the main body 6111′ is relatively large is weak and easily broken; at the same time, it can avoid affecting the adhesion of the electrode patch 100.

The six conductive cores 61130′ of the conductive plate 6113′ are arranged at intervals, and a gap C is formed between two adjacent conductive cores 61130′. The four conductive cores 61130′ located in adjacent rows are arranged in two intervals, and the four intervals C between the four conductive cores 61130′ are arranged in a “cross” shape. The size of the space C between two adjacent conductive cores 61130′ in the same row is greater than the size of the space C between two conductive cores 61130′ in the same row. Seven intervals C are formed between the six conductive cores 61130′, and the seven intervals C are generally connected in the shape of “+”. Adjacent intervals C are also provided in a continuous state. Among the seven intervals C, the straight line of the three intervals C located between two adjacent conductive cores 61130′ in the same line is consistent with the extending direction of the wiring portion 6112′.

Two pairs of pads 6114′ are also provided on the main body 6111′ to be soldered to the two temperature sensors 614′ respectively. Each pair of pads 6114′ is located at the corresponding communication area of 4 intervals 1C formed by the intervals of 4 conductive cores 61130′ located in adjacent rows. The straight line where the line connecting the symmetrical centers of the two pairs of pads 6114′ is consistent with the extending direction of the connecting portion 6112′. The line connecting the two symmetrical centers of the two pairs of pads 6114′ coincides with the longitudinal axis of the main body 6111′. The straight line where the two symmetrical centers of the two pairs of pads 6114′ is located coincides with the longitudinal axis of the conductive pad 6113′. The four conductive cores 61130′ in the first row and the middle are arranged symmetrically to the center, and the four conductive cores 61130′ in the middle row and the last row are also arranged symmetrically to the center. The two pairs of pads 6114′ are arranged in a shape deviated from the symmetrical center of the four conductive cores 61130′ located in two adjacent rows. Specifically, a pair of solder pads 6114′ is provided on the side away from the connecting portion 6112′ from the symmetrical center of the rectangle formed by the four conductive cores 61130′ located in the first row and the middle row. The other pair of pads 6114′ is located on the side of the symmetrical center of the rectangle formed by the four conductive cores 61130′ in the middle row and the last row, which is close to the wiring portion 6112′.

Since the insulated electrodes 600, 600′ of the present disclosure use separate electrode units 610, 610′ to apply an alternating voltage to the patient's tumor site, when it fails to work normally, only the insulated electrodes 600 with separate electrode units 6110, 610′ need to be replaced, 600′, and there is no need to scrap the entire insulated electrode including multiple electrode units 610, 610′, which can reduce the cost of tumor treatment for patients. In addition, the insulated electrodes 600, 600′ of the present disclosure can be freely combined in quantity according to the patient's tumor site and the size of the patient's tumor site, so as to ensure the coverage area of the insulated electrodes 600, 600′ for tumor electric field therapy, and ensure the intensity of electric field therapy; and more The relative position between the insulated electrodes 600 and 600′ can also be freely adjusted according to the patient's own physical differences, tumor location, and tumor size, so as to obtain the optimal electric field intensity and electric field coverage area for tumor treatment, and at the same time allow the application of insulated electrodes The skin on the patient's body surface at 600 and 600′ can breathe freely, avoiding the accumulation of heat on the patient's body surface due to long-term tumor electric field therapy, which cannot be dissipated in time, causing sweating to block pores and produce skin inflammation. At the same time, the flexible circuit board 61 of the insulated electrode 600 of this embodiment is only provided with a first conductive trace L1 electrically connected to the dielectric element 613, which is common to the ground terminals (not shown) of the two temperature sensors 614. One second conductive trace L2 that is electrically connected and two third conductive traces L3 that are electrically connected to the signal ends (not shown) of the two temperature sensors 614 respectively, so as to realize the electric field generator (not shown) shown) is transmitted to the dielectric element 613 through the first conductive trace L1 to achieve the purpose of applying an alternating voltage to the tumor site of the patient for tumor treatment; at the same time, it passes through the second conductive trace L2 and the third conductive trace The line L3 is electrically connected to the two temperature sensors 614 respectively to realize the signal transmission between the electric field generator (not shown) and the two temperature sensors 614, the difficulty of wiring design is low, the structure is simple, the manufacturing process is simplified, and the production is easy, and the product The manufacturing yield is high, which can greatly reduce the manufacturing cost.

### Seventh Embodiment of Insulated Electrodes

35 to 39 show an implementation of the insulated electrode 700 of this embodiment. The insulated electrode 700 includes an electrical connector 72 electrically connected to an electric field generator (not shown) or an adapter (not shown). And a plurality of electrode plates 71 detachably assembled on the electrical connector 72. The electrode plate 71 in this embodiment can also be the insulated electrodes 600, 600′ of the sixth embodiment above. A plurality of electrode plates 71 of the insulated electrode 700 are assembled on the electrical connector 72 in a detachable manner, and the plurality of electrode plates 71 are connected to the electrical connector 72 in parallel, and one of the electrode plates 71 can When damaged and unable to work, it is easy to replace the damaged electrode plate 71 without scrapping multiple electrode plates 71, which can reduce manufacturing costs, avoid waste, and ensure that it has sufficient electric field strength when performing tumor electric field therapy; at the same time, more Each electrode plate 71 can also be freely combined in quantity and freely adjusted in position according to the patient's body difference, tumor site, tumor size, etc., to ensure that the electric field intensity applied to the patient's tumor site is the most suitable; in addition, multiple electrode plates The application position of 71 and the interval between them can also be freely adjusted according to the patient's own situation, which can ensure that the skin of the tumor part of the patient can breathe freely, and avoid the sticking of the electrode plate 71 on the tumor part of the patient due to long-term electric field treatment. The heat generated by the parts accumulates quickly and cannot be dissipated in time, which causes the body surface of the patient to apply the electrode plate 71 to sweat, blocks pores and causes skin inflammation.

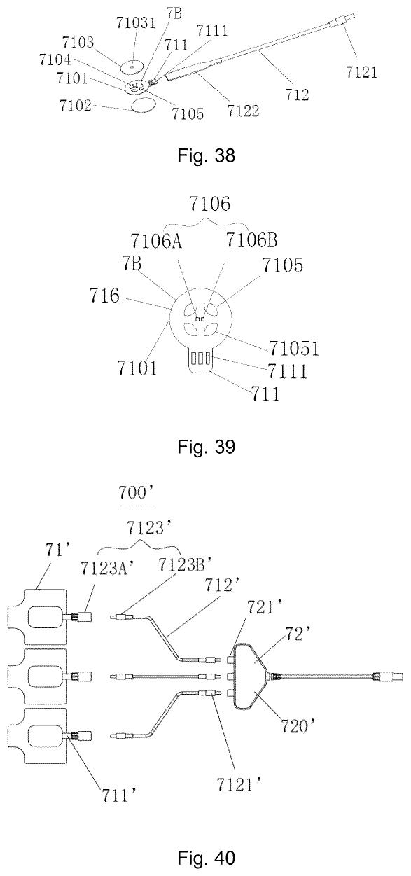

Please refer to FIG. 37 to FIG. 39, a plurality of electrode plates 71 can be detachably plugged into the corresponding parts of the electrical connector 72, so as to realize the parallel connection between each electrode plate 71 and the electrical connector 72, and can The electrical connection with the electric field generator (not shown) is realized through the electrical connector 72, which can be replaced in time and conveniently when a certain electrode plate 71 is damaged, ensuring sufficient electric field strength applied to the tumor site and improving the therapeutic effect. Each electrode plate 71 includes a single electrode unit 710 for applying an alternating electric field to the patient's tumor site, a wiring portion 711 electrically connected to the electrode unit 710, a first wire 712 welded to the wiring portion 711, and a wire attached to the electrode unit 710. The backing 713, the supporting member 714 adhered on the backing 713 in a shape surrounding the electrode unit 710, and the adhesive member 715 covering corresponding parts of the electrode unit 710 and the supporting member 714. One end of the first wire 712 is welded to the wiring portion 711, and the other end is detachably inserted into the electrical connector 72 through the first plug 7121 provided at the end, so as to realize the electrical connection between the electrode unit 710 and the electrical connector 72, and then transmit the alternating electrical signal generated by the electric field generator (not shown) to the electrode unit 710 through the electrical connector 72 to perform electric field therapy on the tumor. Optionally, the electrode plate 71 can be plugged directly with the electric field generator (not shown) through the first plug 7121 of the first wire 712 or plugged with the adapter (not shown) first, and then through the adapter (not shown). not shown) is electrically connected to the electric field generator (not shown) to realize the electrical connection between the electrode plate 71 and the electric field generator (not shown).

The electrode unit 710 of the electrode piece 71 includes a main body portion 7101 which is arranged at the end of the connecting portion 711 and is electrically connected with the connecting portion 711, an insulating plate 7102 and a dielectric element 7103 respectively arranged on the opposite sides of the main part 7101 and arranged on the temperature sensor 7104 on the main body 7101 and on the same side as the dielectric element 7103. The electrode unit 710 has a circular sheet structure as a whole. The main body 7101, the insulating plate 7102 and the dielectric element 7103 are all in the shape of a circular sheet, and the dimensions of the three are approximately the same, and they are arranged in one-to-one correspondence along the thickness direction. The centers of the main body 7101, the insulating plate 7102 and the dielectric element 7103 are located on the same straight line.

The side of the main body 7101 facing the human skin is provided with a conductive plate 7105, and the conductive plate 7105 is welded to the dielectric element 7103 to assemble the dielectric element 7103 on the main body 7101. The conductive plate 7105 can be completely covered by the dielectric element 7103 so that the conductive plate 7105 and the dielectric element 7103 can be welded with solder (not shown). The center of the conductive plate 7105 is located on the centerline of the main body 7101. The conductive plate 7105 includes a plurality of conductive cores 71051 symmetrically arranged around the center, which can effectively prevent the positional displacement of the dielectric element 7103 caused by the accumulation of solder (not shown) during the welding process. The top surfaces of the plurality of conductive cores 71051 are located on the same plane, which can avoid false welding with the dielectric element 7103 during welding. A pair of pads 7106 are also provided between the plurality of conductive cores 71051, which can be welded to corresponding parts of the temperature sensor 7104 to realize electrical connection between the temperature sensor 7104 and the main body 7101. The two pads 7106 include a first pad 7106A and a second pad 7106B. The temperature sensor 7104 has a signal terminal (not shown) and a ground terminal (not shown). The first pad 7106A is soldered to the ground terminal (not shown) of the temperature sensor 7104, and the second pad 7106B is soldered to the signal terminal (not shown) of the temperature sensor 7104.

The insulating plate 7102 is made of insulating material. Preferably, the insulating board 7102 is an epoxy glass cloth laminated board. The insulating plate 7102 is adhered to the side of the main body 7101 away from the skin of the human body through a sealant (not shown). Welding plane; on the other hand, the water vapor in the air on the side away from the skin of the electrode plate 71 can also be in contact with the solder (not shown) between the main body 7101 and the dielectric element 7103, so as to avoid water vapor from corroding the main body 7101 and the dielectric. The solder (not shown) between the components 7103 affects the electrical connection between the main body 7101 and the dielectric component 7103. The size of the insulating plate 7102 is approximately the same as the size of the main body 7101, so as to prevent the insulating plate 7102 from being stuck on the side of the main body 7101 away from the skin of the human body through a sealant (not shown), and the sealant (not shown) creeps through the capillary effect. To the side of the main body 7101 facing the skin of the human body, it affects the filling of the sealant (not shown) in the gap (not shown) formed by welding the dielectric element 7103 and the main body 7101, resulting in the memory of the sealant (not shown) In the cavity, and then avoid the sealant (not shown) curing at high temperature due to the large difference in thermal expansion coefficient between the water vapor in the cavity and the sealant (not shown), which will cause the rapid expansion of water vapor, resulting in bursting, popcorn phenomenon, and damage to the product.

The dielectric element 7103 is made of high dielectric constant material, which has a conductive property of blocking the conduction of direct current and allowing the passage of alternating current, which can ensure the safety of users during tumor electric field treatment. Preferably, the dielectric element 7103 is a dielectric ceramic sheet. A through hole 71031 corresponding to a pair of pads 7106 of the main body 7101 is formed in the middle of the dielectric element 7103 for accommodating the temperature sensor 7104. A metal layer (not shown) is attached to the side of the dielectric element 7103 facing the main body 7101. A point-to-face welding is formed between the metal layer (not shown) of the dielectric element 7103 and the conductive core 71051 of the conductive plate 7105 of the main body 7101, which does not require high welding alignment accuracy, and the welding is more convenient. The inner edge of the metal layer (not shown) of the dielectric element 7103 and the edge of the through hole 71031 of the dielectric element 7103 are arranged at intervals, which can avoid the gap between the metal layer (not shown) of the dielectric element 7103 and the main body 7101. When the solder (not shown) between them diffuses toward the through hole 71031 of the dielectric element 7103 when heated and melted, the temperature sensor 7104 is short-circuited. The outer edge of the metal layer (not shown) of the dielectric element 7103 and the outer edge of the dielectric element 7103 are also arranged at intervals, which can avoid the gap between the metal layer (not shown) and the main body of the dielectric element 7103. When the solder (not shown) between 7101 is heated and melted, it overflows to the outside of the main body 7101, so that when the electrode piece 71 is attached to the body surface of the patient's tumor, the direct current that is not hindered by the dielectric element 7103 passes through and acts on the patient's body. surface.

The gap (not shown) formed by welding the dielectric element 7103 and the main body 7101 is filled with a sealant (not shown) to protect the solder (not shown) between the dielectric element 7103 and the main body 7101 to avoid dielectric The element 7103 is affected by the external force and causes the welding part to break, thereby causing the alternating electric field to be unable to be applied to the patient's tumor site through the dielectric element 7103; at the same time, it can also prevent water vapor in the air from entering the gap (not shown) to corrode the dielectric element 7103 and The solder (not shown) between the main parts 7101 further affects the electrical connection between the dielectric element 7103 and the main part 7101. The outer diameter of the dielectric element 7103 is slightly smaller than the diameter of the main body 7101, so that the sealant (not shown) can pass through the capillary along the edge of the main body 7101 outside the dielectric element 7103 when filling the sealant (not shown). The phenomenon fills the gap (not shown), which is beneficial to the filling of the sealant (not shown) in the gap (not shown) formed by welding the dielectric element 7103 and the main body 7101. When the gap (not shown) formed by welding the dielectric element 7103 and the main body 7101 is filled with sealant (not shown), the air in the gap (not shown) can be discharged from the perforation 71031 of the dielectric element 7103 to avoid The sealant (not shown) filled in the gap (not shown) creates voids to improve product quality.

The temperature sensor 7104 is provided on the main body by welding its ground terminal (not shown) to the first pad 7106A provided on the main body 7101 and its signal terminal (not shown) to the second pad 7106B provided on the main body 7101 Section 7101 on. The temperature sensor 7104 is accommodated in the through hole 71031 of the dielectric element 7103 after being welded on the main body 7101. Preferably, the temperature sensor 7104 is a thermistor. The temperature sensor 7104 is used to monitor the temperature of the sticker 715 covering the side of the dielectric element 7103 of the electrode unit 710 facing the human skin, and further detect the temperature of the human skin attached to the sticker 715. When the temperature detected by the temperature sensor 7104 exceeds the upper limit of the safe temperature of the human body, the tumor electric field therapy system can promptly reduce or turn off the alternating voltage applied to the electrode pad 71 by the electric field generator (not shown), so as to avoid low-temperature burns on the human body. The temperature sensor 7104 is welded to the main body 7101 through a pair of pads 7106 of the main body 7101 and then sealed with a sealant (not shown) to prevent water vapor from corroding the temperature sensor 7104 and causing the temperature sensor 7104 to fail.

The connection portion 711 is laterally extended from the main body portion 7101 of the electrode unit 710. A heat-shrinkable sleeve 7122 is wrapped around the welding portion of the connecting portion 711 and the first wire 712. The heat-shrinkable sleeve 7122 insulates and protects the connection between the first wire 712 and the wiring portion 711, and provides support to avoid breakage at the connection between the first wire 712 and the wiring portion 711, and is also dustproof and waterproof.

The main body portion 7101 of the electrode unit 710 and the connection portion 711 together constitute the flexible circuit board 716 of the electrode plate 71. From the perspective of the formation of the electrode unit 710, the insulating plate 7102 is arranged on the side of the main body 7101 of the flexible circuit board 716 away from the human skin, the dielectric element 7103 is arranged on the side of the main body 7101 of the flexible circuit board 716 facing the human skin, and the temperature sensor 7104 is disposed on the side of the main body 7101 of the flexible circuit board 716 facing the human skin.

The flexible circuit board 716 is composed of an insulating substrate 7B and multiple conductive traces (not shown) embedded in the insulating substrate 7B. Multiple conductive traces (not shown) in the insulating substrate 7B are formed. Multiple conductive traces (not shown) in the insulating substrate 7B of the main body portion 7101 are electrically connected to corresponding multiple conductive traces (not shown) in the insulating substrate 7B of the connecting portion 711. In this embodiment, the flexible circuit board 716 has three conductive traces (not shown), including a conductive trace (not shown) that connects all the conductive cores 71051 of the conductive plate 7105 on the main body 7101 in series, and a conductive trace (not shown) A conductive trace (not shown) electrically connected to the ground terminal (not shown) of the temperature sensor 7104 on the main body 7101 and a signal terminal (not shown) electrically connected to the temperature sensor 7104 on the main body 7101 Conductive traces (not shown). Three golden fingers 7111 are provided on the side of the connecting portion 711 facing the human skin. The three golden fingers 7111 of the connecting portion 711 are respectively electrically connected to three conductive traces (not shown). The three gold fingers 7111 of the wiring portion 711 are welded to the end of the first wire 712 far away from the first plug 7121 to realize the electrical connection between the main body 7101 of the electrode unit 710 and the first wire 712, and further realize the dielectric through the main body 7101. The electrical connection between the element 7103 and the temperature sensor 7104 and the first wire 712.

The conductive core 71051 is exposed on the insulating substrate 7B of the main body 7101. The insulating substrate 7B of the flexible circuit board 716 can isolate the water vapor in the air around the electrode piece 71 and the solder (not shown) between the conductive plate 7105 and the dielectric element 7103, so as to avoid the water vapor in the air on the side away from the skin from corroding the device. Solder (not shown) between the conductive pad 7105 and the dielectric element 7103 on the main body portion 7101 of the flexible circuit board 716. The insulating substrate 7B of the flexible circuit board 716 and the insulating plate 7102 play a double isolation role, which can prolong the service life of the electrode plate 71.

The backing 713 is arranged in sheet form, which is mainly made of flexible and breathable insulating material. The backing 713 is a mesh fabric. Specifically, the backing 713 is a net-like non-woven fabric, which is soft, light, moisture-proof, and breathable. It can keep the patient's skin surface dry after long-term sticking on the patient's body surface. The side of the backing 713 facing the patient's body surface is also coated with a biocompatible adhesive (not shown), which is used to closely adhere the backing 713 to the corresponding body surface of the patient's tumor site. In this embodiment, only one electrode unit 710 is glued on the backing 713. The backing 713 is generally in the shape of a cube sheet. The four corners of the backing 713 are rounded.

The supporting member 714 is adhered to the backing 713 and surrounds the outer side of the electrode unit 710. A through hole 7141 is formed in the middle of the support member 714 for accommodating the electrode unit 710. The support member 714 may be made of foam material. The support member 714 is flush with the surface of the electrode unit 710 away from the backing 713. That is, the supporting member 714 is flush with the surface of the electrode unit 710 facing the sticking member 715.

Sticker 715 has double-sided adhesive. One side of the adhesive member 715 is glued on the support member 714 and the surface of the electrode unit 710 away from the backing 713. The other side of the sticker 715 is used as an application layer, which is applied on the surface skin of the human body to keep the skin surface moist and relieve local pressure. Preferably, the adhesive part 14 can use conductive hydrogel to serve as a conductive medium. Under the support of the supporting member 714, the sticker 715 has better adhesion to human skin.

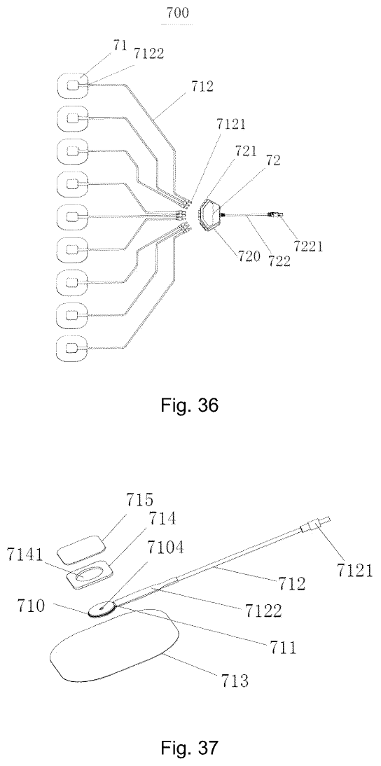

Please refer to FIG. 36 to FIG. 37, the electrical connector 72 is provided with a plurality of sockets 721 plugged with the first plugs 7121 of the first wires 712 of the corresponding electrode plates 71 and an adapter (not shown).) or a second wire 722 plugged into an electric field generator (not shown). The end of the second wire 722 away from the electrical connector 72 is provided with a second plug 7221, which can be plugged directly with an electric field generator (not shown) or plugged with an adapter (not shown) first, and then through the adapter (not shown) plugged with the electric field generator (not shown) to realize the electrical connection between it and the electric field generator (not shown). The plurality of sockets 721 and the second wires 722 are respectively disposed on opposite ends of the electrical connector 72. The electrical connector 72 is plugged with the first plug 7121 of the first wire 712 of the electrode plate 71 through its socket 721, so as to connect the plurality of electrode plates 71 to the electrical connector 72 respectively to realize the connection between the plurality of electrode plates 71 and the electrical connector. 72, and then through the second plug 7221 plugged with the electric field generator (not shown) or adapter (not shown), a plurality of electrode plates 71 and the electric field generator (not shown) Shown) electrical connection between. When in use, a plurality of electrode plates 71 are attached to the corresponding body surface of the patient's tumor site, and the plurality of electrode plates 71 are inserted into the corresponding socket 721 of the electrical connector 72 through its first plug 7121, and the electrical connector 72 is inserted into the corresponding socket 721 through its second plug 7221. Electrically connect the electric field generator (not shown), so that the alternating electric field generated by the electric field generator (not shown) is transmitted to the plurality of electrode plates 71 through the electrical connector 72, and acts on the electrode plate 71 through the plurality of electrode plates 71. The patient's tumor site to interfere or prevent the mitosis of the patient's tumor cells, so as to achieve the purpose of treating the tumor. The electrode plate 71 of the insulated electrode 700 of the present disclosure and its electrical connector 72 are detachably plugged in. The electrode plate 71 only includes one electrode unit 710, and each electrode unit 710 is electrically connected to the electrode unit 710 through a first wire 712. The electric field generator (not shown) is electrically connected. When the electrode unit 710 is damaged or the first wire 712 is damaged and cannot work, only the corresponding electrode piece 71 needs to be replaced, which can reduce the cost of tumor treatment for patients. The number of electrode pieces 71 of the insulated electrode 700 can also be freely combined according to the tumor location and size of the patient, so as to ensure the coverage area of the insulated electrode 700 for tumor electric field therapy, ensure the electric field strength of the insulated electrode 700 for tumor electric field therapy, and improve the effect of tumor electric field therapy. In addition, the relative positions of the plurality of electrode plates 71 of the insulated electrode 100 can also be freely adjusted according to the patient's own physical differences, tumor location, and tumor size, so as to obtain the optimal electric field intensity and electric field coverage area for tumor treatment, and at the same time allow the application The skin on the patient's body surface of the electrode plate 71 can breathe freely, avoiding the accumulation of heat on the patient's body surface due to long-term tumor electric field treatment and unable to dissipate it in time, causing sweating to block pores and produce skin inflammation.

In this embodiment, the number of sockets 721 of the electrical connector 72 is nine, and the number of electrode plates 71 is nine. The electrical connector 72 is provided with a body 720, and the body 720 is substantially polyhedral. In this embodiment, the body 720 is roughly in the shape of a hexagonal prism. The nine sockets 721 are respectively arranged on a plurality of adjacent side surfaces of the body 720, and an obtuse angle is formed between adjacent side surfaces. The second wire 722 is disposed on a side of the body 720 away from the socket 721. In this embodiment, the nine sockets 721 are equally arranged on three adjacent side surfaces of the main body 720, and every three sockets 721 are arranged on the same side surface of the main body 720 of the electrical connector 72. The terminals (not shown) in the nine sockets 721 of the electrical connector 72 can be connected in series, so that the nine electrode plates 71 are connected in series. Terminals (not shown) in the nine sockets 721 of the electrical connector 72 may also be connected in parallel, so that the nine electrode plates 71 are connected in parallel. When the terminals (not shown) in the socket 721 of the electrical connector 72 are connected in series, all the electrode plates 71 need to be plugged into the electrical connector 72 for use. When the terminals (not shown) in the socket 721 of the electrical connector 72 are connected in parallel, part of the electrode plates 71 can be selected and plugged into the electrical connector 72 as required, which will be more convenient and flexible in use. Optionally, terminals (not shown) in the nine sockets 721 of the electrical connector 72 may be partially connected in series and partially connected in parallel. The terminals (not shown) in the socket 721 of the electrical connector 72 can be connected in series or in parallel or partly in series or partly in parallel as required, so that all of the plurality of electrode plates 71 connected to the electrical connector 72 are connected in series or in parallel Or partly in series and partly in parallel. When the size of the tumor is too large, an appropriate number of electrode plates 71 can be selected and the interval between the electrode plates 71 can be freely adjusted to ensure the coverage area of the insulated electrode 700 for tumor electric field therapy and the effect of electric field therapy. When the position of the tumor deviates to the side of the corresponding part of the body, the corresponding body surface away from the tumor can appropriately increase the number of electrode pads 71 of the insulated electrode 700 to enhance the electric field strength on the side away from the tumor.

### Variation of Insulated Electrode in Seventh Embodiment

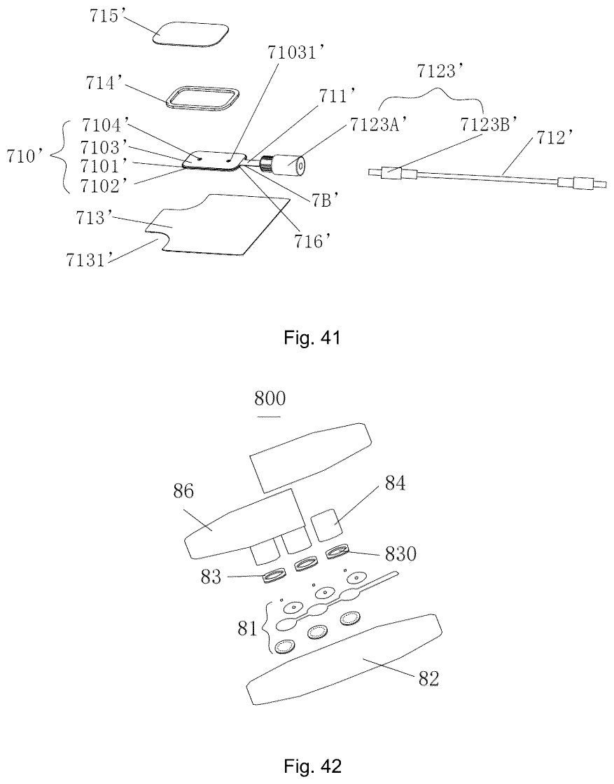

FIG. 40 and FIG. 41 disclose the insulated electrode 700′ shown in the variant embodiment of the seventh embodiment, which also includes a plurality of electrode plates 71′ for applying an alternating electric field to the patient's tumor site and an adapter (not shown in the figure). shown) or an electrical connector 72′ electrically connected to an electric field generator (not shown). A plurality of electrode plates 71′ are detachably assembled on the electrical connector 72′ to realize the electrical connection between them and the electrical connector 72′, and then realize the contact with the electric field through the electrical connector 72′. Electrical connections between generators (not shown). Each electrode plate 71′ includes an electrode unit 710′, a connection part 711′ connected to the electrode unit 710′, a first wire 712′ welded to the connecting portion 711′, and a backing 713′ pasted to the electrode unit 710′, a support member 714′ surrounding the electrode unit 710′ and glued on the backing 713′, and an adhesive member 715′ covering corresponding parts of the electrode unit 710′ and the support member 714′. The insulated electrode 700′ is different from the insulated electrode 700 of the first embodiment in that: the insulated electrode 700′ includes three electrode pieces 71′, the body 720′ of the electrical connector 72′ is roughly in the shape of a triangular prism, and the electrical connector 72′ is provided with three sockets 721′, and the three sockets 721′ are all arranged on the same side of the body 720′ of the electrical connector 72′. The connecting portion 711′ of each electrode piece 71′ is connected to the corresponding first wire 712′ in a detachable plug-in manner. The connection portion 711′ of the electrode plate 71′ is electrically connected to the first wire 712′ through a connector 7123′. The connector 7123′ includes a mating socket 7123A′ and a mating plug 7123B′. The docking socket 7123A′ is connected to the wiring portion 711′, and the docking plug 7123B′ is connected to the end of the first wire 712′ away from the first plug 7121′. Also, the docking socket 7123A′ is arranged at the end of the wiring portion 711′, and the docking plug 7123B′ is arranged at the end of the first wire 7121′ away from the first plug 7121′. The docking socket 7123A′ and the electrode unit 710′ are respectively located at opposite ends of the connection portion 711′. The docking plug 7123B′ and the first plug 7121′ are respectively disposed at opposite ends of the first wire 712′. When the electrode unit 710′ of the electrode plate 71′ cannot work due to damage, only the part of the electrode plate 71′ except the first lead 712′ can be replaced, and the first lead 712′ can continue to be used, further reducing the cost of the patient's tumor treatment.

The backing 713′ of the electrode plate 71′ is roughly in a “convex” shape. The backing 713′ has two concave corners 7131′ which are respectively inwardly recessed from two corners thereof. The two concave corners 7131′ are respectively located at the two corners of the backing 713′ away from the connecting portion 711′. The concave corner 7131′ of the corner of the backing 713′ communicates with the outside and is arranged in an “L” shape. The angle between the two sides of the backing 713′ forming the concave angle 7131′ is greater than or equal to 90 degrees, so as to prevent the corners of the backing 713′ from arching when the electrode plate 71′ is applied on the body surface corresponding to the patient's tumor site. The folds prevent air from entering between the electrode unit 710′ and the skin from the folds to increase the impedance between the electrode unit 710′ and the skin, thereby causing heat increase of the electrode unit 710′ and resulting in low-temperature burns.

The electrode plate 71′ is similar to the insulated electrode 600′ in the variant embodiment of the sixth embodiment, and its electrode unit 710′ is roughly in the shape of a rectangular sheet. The main body 7101′, the insulating plate 7102′ and the dielectric element 7103′ of the electrode unit 710′ are all rectangular sheet structures. Two temperature sensors 7104′ are provided on the side of the main body 7101′ where the dielectric element 7103′ is disposed. The dielectric element 7103′ is provided with two through holes 71031′ for accommodating the temperature sensor 7104′ respectively. The two temperature sensors 7104′ are symmetrically arranged on the main body portion 7101′, which can detect the temperature of human skin corresponding to different positions and ensure the accuracy of the detection data. Four conductive traces (not shown) are embedded in the insulating substrate 7B′ of the flexible circuit board 716′ formed by the main body portion 7101′ of the electrode unit 710′ and the connection portion 711′. The four conductive traces (not shown) of the flexible circuit board 716′ are respectively one conductive trace (not shown) that connects all the conductive cores (not shown) of the conductive plate (not shown) located in the main body 7101′ in series. shown), one conductive trace (not shown) that connects the ground terminals (not shown) of the two temperature sensors 7104′ on the main body 7101′ in series, and two lines that connect the signal terminals of the two temperature sensors 7104′ in parallel (not shown) conductive traces (not shown). The connection portion 711′ is provided with four gold fingers (not shown) on the side facing the human skin. The four conductive traces (not shown) are respectively electrically connected to the four gold fingers (not shown) of the connecting portion 711′.

At least one electrode plate 71, 71′ of the insulated electrodes 700, 700′ of this embodiment is detachably plugged into an electric field generator (not shown) through the first wire 712, 712′ provided thereon, or detachable first. The ground is plugged into the adapter (not shown), and then electrically connected to the electric field generator (not shown) through the adapter (not shown), or detachably plugged into the electrical connector 72, 72′, and then electrically connected to the electric field generator (not shown) through electrical connectors 72, 72′ to realize the electrical connection between it and the electric field generator (not shown), and each electrode plate 71, 71′ only includes one electrode unit 710, 710′ electrically connected to the corresponding first wire 712, 712′, when the electrode unit 710, 710′ is damaged and unable to work, only the corresponding electrode plate 71, 71′ needs to be replaced, which can reduce the cost of cancer treatment for patients. In addition, the insulated electrodes 700, 700′ of this embodiment can be freely combined in number or freely adjusted in the position of the electrode plates 71, 71′ according to the patient's tumor location, tumor location, and tumor size, so as to ensure that the insulated electrodes 700, 700′ The coverage area of the tumor electric field treatment ensures the electric field intensity of the insulated electrodes 700, 700′ for tumor electric field treatment, and improves the effect of the electric field treatment; at the same time, the relative interval between the electrode plates 71, 71′ allows the patient's body surface skin to breathe freely. External air conducts heat exchange to avoid skin diseases caused by sweating and clogged pores caused by heat accumulation on the body surface of patients undergoing electric field treatment for a long time.

### Eighth Embodiment of Insulated Electrodes

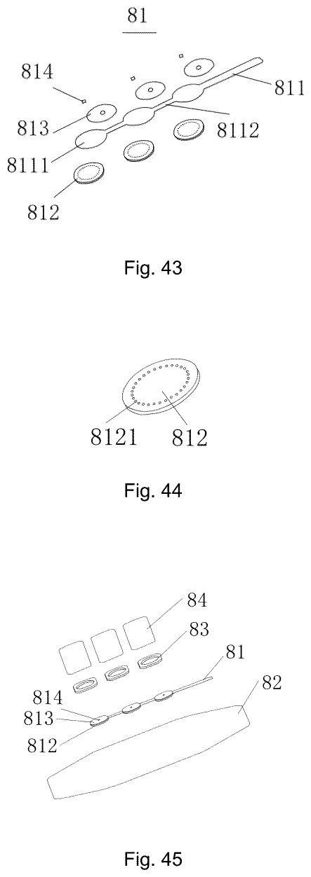

Please refer to FIG. 42 to FIG. 45, the insulated electrode 800 includes an electrical functional assembly 81, a flexible backing 82, a number of supports 83, a number of stickers 84, and an adhesive that is arranged above the stickers 84 and can be bonded to the backing 82. Release paper 86. The electrical function component 81 includes a flexible circuit board 811, a heat dissipation reinforcing member 812 and a dielectric element 813 respectively disposed on opposite sides of the flexible circuit board 811, and a temperature sensor 814. The flexible circuit board 811 has several main parts 8111 and several connecting portions 8112 connected with the main parts 8111. The specific structure of the electrical functional assembly 81 is basically the same as the electrical functional assembly in the previous embodiment, the only difference is that the insulating plate in the previous embodiment is replaced by the heat dissipation reinforcing member 812 to improve the heat dissipation performance, the following is only for the heat dissipation reinforcing member 812 For illustration, other structures of the electrical functional assembly 81 will not be described repeatedly.

The heat dissipation reinforcing member 812 is generally arranged in a circular sheet shape, and its side close to the patient's body surface is pasted on the main body portion 8111 of the flexible circuit board 811 through adhesive (not shown), and its side away from the patient's body surface Attached to the backing 82 by the biocompatible adhesive provided on the backing 82. The heat dissipation reinforcing part 812 is arranged on the side of the main body 8111 of the flexible circuit board 811 away from the patient's body surface, and is used to support the main body 8111 of the flexible circuit board 811, so as to facilitate welding the temperature sensor 814 and the dielectric element 813 to the flexible circuit on the main body portion 8111 of the board 811. The heat dissipation reinforcing member 812 is sandwiched between the backing 82 and the main body portion 8111 of the flexible circuit board 811 after the electrical functional assembly 81 is assembled on the backing 82. The diameter of the heat dissipation reinforcing member 812 is substantially the same as the diameter of the main body portion 8111 of the flexible circuit board 811. The quantity of the heat dissipation reinforcing pieces 812 is consistent with the quantity of the main body 8111 of the flexible circuit board 811. The number of the heat dissipation reinforcing pieces 812 is consistent with the number of the dielectric elements 813. The heat dissipation reinforcing member 812 is insulated from the conductive plate (not shown) on the main body portion 8111 of the flexible circuit board 811. The heat dissipation reinforcing member 812 is electrically insulated from the flexible circuit board 811.

The heat dissipation reinforcing member 812 is made of a material with a thermal conductivity greater than 200 W/mK, which can quickly expose the main body 8111 of the flexible circuit board 811 for applying an alternating electric field to the conductive plate (not shown in the figure) on the side close to the patient's body surface. shown) and the heat generated by the dielectric element 813 and the heat accumulated on the patient's body surface due to the long-term application of an alternating electric field to the patient's tumor body surface through the insulated electrode 800 is dissipated, enhancing the performance of the insulated electrode 800 in the tumor electric field treatment process The heat dissipation performance can ensure that the treatment time can be extended while keeping the magnitude of the alternating electric field applied to the insulated electrode 800 constant, so that the alternating electric field therapy for tumors has a good therapeutic effect.

The heat dissipation reinforcing member 812 can be a metal plate, a metal alloy plate or a graphene composite plate. One or more heat dissipation holes 8121 are also provided on the heat dissipation reinforcing member 812, which can further quickly dissipate heat through the backing 82. The heat dissipation holes 8121 are evenly distributed on the heat dissipation reinforcing member 812. The cooling holes 8121 are arranged in a circular shape. Preferably, the heat dissipation reinforcing member 812 is a metal plate or a metal alloy plate with a thickness of 0.1-0.7 mm. Specifically, the heat dissipation reinforcing member 812 is an aluminum plate or an aluminum alloy plate with a thickness of 0.3-0.6 mm. Specifically, the heat dissipation reinforcing member 812 is an aluminum plate with a thickness of 0.6 mm. The number of cooling holes 8121 uniformly distributed on the aluminum plate is 30, and the diameter of the cooling holes 8121 is 0.5 mm. Alternatively, the heat dissipation reinforcing member 812 is a 6063 type aluminum alloy plate with a thickness of 0.3 mm. The thermal conductivity of 6063 aluminum alloy is 201 W/mK. The number of heat dissipation holes 8121 evenly distributed on the 6063 type aluminum alloy plate is 50, and the diameter of the heat dissipation holes 8121 is 0.4 mm. Alternatively, the heat dissipation reinforcing member 812 is made of graphene composite material with a thickness of 0.1 mm. The thermal reinforcement member 812 has a thermal conductivity greater than 300 W/mK.

The backing 82 is arranged in sheet form, which is mainly made of flexible and breathable insulating material. The backing 82 has some ventilating holes (not shown) that are set through, which can make the hair follicles and sweat glands of the skin covered by the backing 82 on the patient's body surface breathe freely when the backing 82 is applied on the patient's body surface, and avoid being backed. The sweat glands and hair follicles on the patient's body surface covered by lining 82 damage the superficial layer of the patient's skin and cause skin inflammation due to blockage. The backing 82 is a mesh fabric. Specifically, the backing 82 is a mesh non-woven fabric. The side of the backing 82 facing the patient's body surface is also coated with a material-compatible adhesive for closely fitting the backing 82 to the body surface of the patient's target area.

The supporting member 83 is substantially in the shape of a hollow ring, and has a through hole 830 for allowing the dielectric element 813 to pass through. The support member 83 has substantially the same thickness as the dielectric element 813. The plane where the top of the support 83 is located is at the same vertical height as the surface of the dielectric element 813 facing the patient's body surface, that is, the surface of the support 83 near the patient's body surface is at the same vertical height as the dielectric element 813 near the patient's body surface. The surfaces of the sides are coplanar. The through hole 830 is arranged in a circular shape, and its diameter is substantially the same as that of the dielectric element 813. The through hole 830 is used to accommodate the dielectric element 813 after the insulating electrode 800 is assembled.

Both the supporting member 83 and the dielectric element 813 are disposed on the same side of the flexible circuit board 811. The support member 83 and the heat dissipation reinforcing member 812 are respectively located on opposite sides of the flexible circuit board 811. The support member 83 is arranged in a sheet shape, and can be made of polyethylene (PE) material or PET material or heat-conducting silica gel sheet or a soft, chemically stable, Made of lightweight, non-deformable and non-toxic insulating material. The supporting member 83 is disposed around the dielectric element 813 for positioning and supporting the sticking member 84, and at the same time can improve the wearing comfort of the insulated electrode 800. The flexible circuit board 811 is interposed between the support member 83 and the heat dissipation reinforcing member 812. In this embodiment, the supporting member 83 can be flexible foam. The side of the support member 83 close to the patient's body surface is attached to the adhesive member 84, and the side of the support member 83 away from the patient's body surface is attached to the backing 82 through the biocompatible adhesive on the backing 82.

The sticker 84 is arranged in a sheet shape, and one side thereof is attached to the side of the support member 83 and the dielectric element 813 close to the patient's body surface. The other side of the sticker 84 is attached to the corresponding part of the release paper 86 when the insulated electrode 800 is not in use, and is attached to the patient's body surface when the insulated electrode 800 is in use, so that the insulated electrode 800 is closely attached to the corresponding body surface of the patient's tumor. The number of sticking pieces 84 is the same as the number of supporting pieces 83.

The release paper 86 is bonded to the other parts of the backing 82 except the electrical functional assembly 81 through the biocompatible adhesive coated on the backing 82, and is used to cover the electrical functional assembly 81 and be located on the electrical functional assembly. The support member 83 and the sticking part 84 on the assembly 81, thereby protecting the biocompatible adhesive on the sticking part 84 and the backing 82, avoiding the contamination of the biocompatible adhesive on the sticking part 84 and the backing 82. Release paper 86 is made of insulating material.

Using a metal plate or metal alloy plate (aluminum metal plate with a thermal conductivity of 237 W/mK) with a thermal conductivity greater than 200 W/mK as the insulating electrode 800 of the heat dissipation reinforcing member 812 has the same area and the same thickness as the heat dissipation reinforcing member 812 And the electrode of the epoxy glass cloth laminated plate with a thermal conductivity of 0.2 W/mK has the same applied electric field, the same electrode application position, and the same treatment time. The temperature rise rate of the patient's skin surface with the electrode of the laminate is about 0.0223° C./s (the temperature test range is 36.5° C. to 39° C.), while the temperature rise rate of the patient's skin surface using the insulated electrode 800 of the present disclosure is about 0.0178° C./s (Temperature test range is 36.5° C. to 39° C.); the temperature rise rate of the electrode using the aluminum metal plate as the heat dissipation reinforcing member 812 is about 20.2% lower than that of the electrode using the epoxy glass cloth laminate in the actual use process.

As verified by the above test, the insulated electrode 800 of this embodiment has a high thermal conductivity by using a heat dissipation reinforcing member 812 with a thermal conductivity greater than 200 W/mK and with uniformly distributed heat dissipation holes 8121 on it, and can be applied for a long time. A higher level of alternating electric field makes the surface temperature of the patient's tumor rise to a certain threshold, and quickly conducts the heat accumulated on the patient's surface skin, so that the tumor electric field therapy has a better therapeutic effect, without the need to quickly reduce the applied The alternating voltage on the electrode is used to reduce the electric field intensity, thereby reducing the surface temperature of the patient's tumor site and reducing the therapeutic effect of the tumor electric field.

### Variant Embodiment of the Eighth Embodiment of the Insulated Electrode

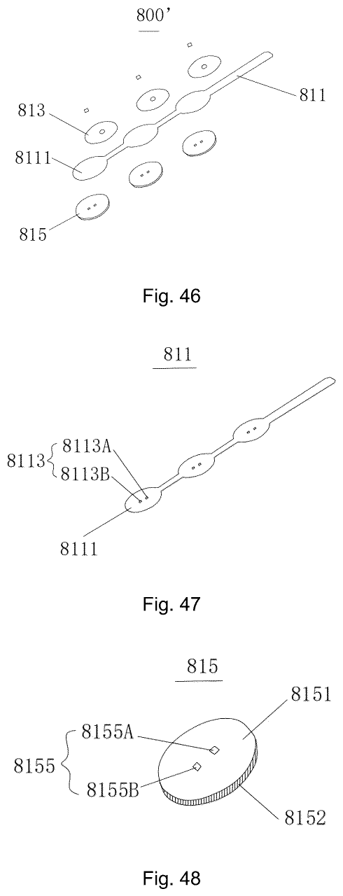

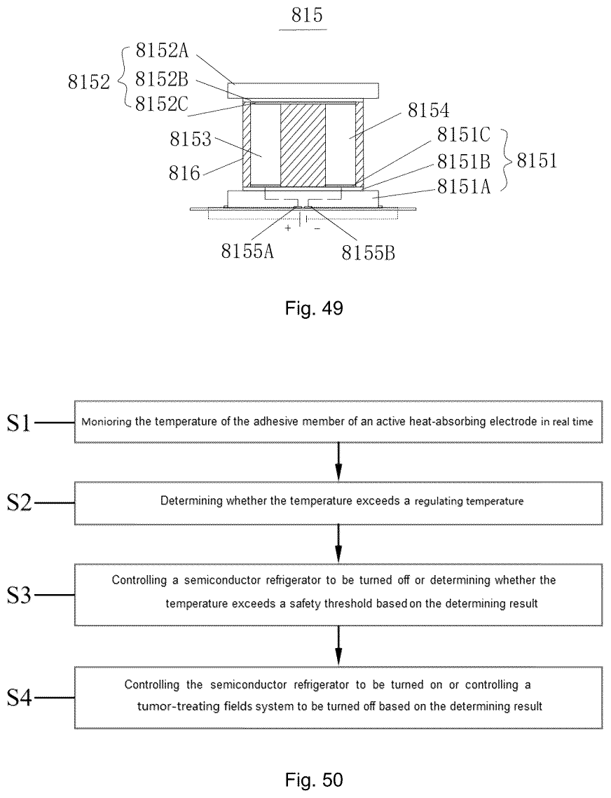

Please refer to FIG. 46 to FIG. 49. The specific structure of the insulated electrode 800′ of this embodiment is basically the same as that of the insulated electrode 800′ in the eighth embodiment. 812, which can further improve the heat dissipation performance. The following only describes the semiconductor refrigerator 815. Other structures of the insulated electrode 800′ follow the label of the insulated electrode ‘800 and refer to the drawing of the insulated electrode ‘800, and will not be repeated here.

The main body portion 8111 of the flexible circuit board 811 has a welding part 8113 exposed on the side away from the patient's body surface. The welding part 8113 includes two first welding parts 8113A and second welding parts 8113B arranged at intervals. The semiconductor cooler 815 is welded to the welding portion 8113 to realize the electrical connection between the flexible circuit board 811 and the semiconductor cooler 815. The semiconductor refrigerator 815 can quickly dissipate the heat accumulated on the patient's skin, avoiding the long-term and continuous application of alternating voltage to the electrically connected flexible circuit board 811 and dielectric element 813, which will cause the patient's tumor site to be damaged. The skin on the corresponding body surface is scalded at low temperature on the patient's tumor site due to heat accumulation.

The semiconductor refrigerator 815 is arranged in a circular sheet shape, and is welded to the welding portion 8113 of the main body portion 8111 of the flexible circuit board 811 to realize electrical connection with the flexible circuit board 811. The semiconductor refrigerator 815 is sandwiched between the main body 8111 of the flexible circuit board 811 and the backing, which can quickly dissipate the heat from the skin part of the patient's body surface where the insulated electrode 800′ is applied. One side of the semiconductor refrigerator 815 is disposed on the main body portion 8111 of the flexible circuit board 811 by welding, and the other side is attached to the dielectric backing 82 through the biocompatible adhesive disposed on the dielectric backing 82. The semiconductor refrigerator 815 has a refrigeration end 8151 close to the main body portion 8111 of the flexible circuit board 811, a refrigeration end 8152 away from the main body portion 8111 of the flexible circuit board 811, and an N-type semiconductor 8153 sandwiched between the refrigeration end 8151 and the refrigeration end 8152. with P-type semiconductor 8154. The semiconductor refrigerator 815 is attached to the dielectric backing 82 through the heat dissipation end 8152. The N-type semiconductor 8153 and the P-type semiconductor 8154 are mainly made of bismuth telluride with impurities added through special treatment. The semiconductor cooler 815 implements electrical conduction between the refrigeration end 8151 and the refrigeration end 8152 through the N-type semiconductor 8153 and the P-type semiconductor 8154.

The refrigeration end 8151 has a soldering pad 8155 corresponding to the soldering portion 8113 on the main body portion 8111 of the flexible circuit board 811. The soldering pads 8155 include a positive soldering pad 8155A soldered to the first soldering portion 8113A of the main body 8111 of the flexible circuit board 811 and a negative soldering pad 8155B soldered to the second soldering portion 8113B of the main body 8111 of the flexible circuit board 811. The refrigeration end 8151 is disposed on the flexible circuit board 811 through the welding pad 8155, and is electrically connected with the flexible circuit board 811 through the welding pad 8155. The refrigeration end 8151 includes a cold-end ceramic sheet 8151A welded to the main body portion 8111 of the flexible circuit board 811, a cold-end heat-conducting member 8151B disposed on the side of the cold-end ceramic sheet 8151A away from the flexible circuit board 811, and a cold-end heat-conducting member 8151B arranged on the cold-end heat-conducting member 8151B. The two cold-end metal conductors 8151C are arranged in parallel and at intervals, and are connected to the N-type semiconductor 8153 and the P-type semiconductor 8154 respectively.

The welding pad 8155 is disposed on the side of the cold-end ceramic sheet 8151A facing the flexible circuit board 811. The ceramic chip 8151A at the cold end is roughly arranged in a circular shape, and its size is slightly smaller than that of the main body 8111 of the flexible circuit board 811. There is a gap (not shown) between the cold-end ceramic chip 8151A and the main body portion 8111 of the flexible circuit board 811 through the welding portion 8113 and the welding pad 8155 after welding. The gap (not shown) is filled with the sealant 816, which can prevent the water vapor from the patient's body surface from entering the gap (not shown) between the cold-end ceramic sheet 8151A and the main body 8111 of the flexible circuit board 811 to erode the welding part and cause a short circuit. It affects the electrical connection between the cold-end ceramic chip 8151A and the flexible circuit board 811. The cold-end ceramic sheet 8151A is sandwiched between the main body 8111 of the flexible circuit board 811 and the cold-end heat conducting member 8151B.

The cold-end heat conduction member 8151B is an integrally arranged circular sheet structure, and is used to fix the cold-end metal conductor 8151C to the cold-end ceramic sheet 8151A. The size of the cold end heat conducting member 8151B is slightly smaller than the size of the cold end ceramic sheet 8151A. The cold-end heat-conducting element 8151B and the main body 8111 of the flexible circuit board 811 are respectively located on opposite sides of the cold ceramic sheet 113. The cold end heat conductor 8151B is made of thermally conductive and non-conductive materials. The cold-end heat-conducting member 8151B can be heat-conducting silica gel. The side of the cold-end heat-conducting member 8151B away from the cold-end ceramic chip 8151A is respectively recessed downward from the top to provide two recessed spaces (not labeled) for accommodating the cold-end metal conductor 8151C. The two recessed spaces (not numbered) are arranged at intervals and roughly in a circular shape.

The two cold-end metal conductors 8151C are respectively disposed in corresponding recessed spaces (not labeled), and protrude from the top of the cold-end heat conducting element 8151B. The parts of the two cold-end metal conductors 8151C protruding from the cold-end heat conducting element 8151B have the same height. That is, the sides of the two cold-end metal conductors 8151C protruding from the cold-end heat conducting member 8151B are at the same level. The dimensions of the two spaced cold-end metal conductors 8151C are exactly the same. The cold-end metal conductor 8151C is roughly arranged in a circular sheet shape, and is made of the same material as the N-type semiconductor 8153 and the P-type semiconductor 8154. Cold end metal conductor 8151C is preferably made of copper. The diameter of the cold-end metal conductor 8151C is approximately the same as the diameter of the recessed space (not numbered) of the cold-end heat conducting element 8151B. Two cold-end metal conductors 8151C arranged at intervals are arranged on the side of the cold-end heat conducting member 8151B away from the flexible circuit board 811. The cold-end heat conducting element 8151B is sandwiched between the cold-end metal conductor 8151C and the cold-end ceramic sheet 8151A. Two corresponding conductive traces (not shown) are provided in the cold-end ceramic chip 8151A and the cold-end heat-conducting element 8151B respectively. One end of the two conductive traces on the cold-end ceramic chip 8151A is respectively connected to the solder pads 8155A and 8155B. Two cold-end metal conductors 8151C arranged at intervals are assembled on the cold-end ceramic sheet 8151A through the cold-end heat conducting member 8151B.

Both the N-type semiconductor 8153 and the P-type semiconductor 8154 are substantially columnar. The N-type semiconductor 8153 and the P-type semiconductor 8154 are respectively assembled on the corresponding cold-end metal conductors 8151C. The diameter of the N-type semiconductor 8153 is the same as that of the corresponding cold-end metal conductor 8151C. The diameter of the P-type semiconductor 8154 is the same as that of the corresponding cold-end metal conductor 8151C. Both the N-type semiconductor 8153 and the P-type semiconductor 8154 are made of copper. The N-type semiconductor 8153 and the P-type semiconductor 8154 have the same thickness.

The heat dissipation end 8152 includes a hot-end ceramic sheet 8152A attached to the backing through a biocompatible adhesive on the backing 82, and a hot end on the side of the hot-end ceramic sheet 8152A away from the backing 82. The heat transfer element 8152B and the hot end metal conductor 8152C disposed on the other side of the hot end heat transfer element 8152B. The hot-end metal conductor 8152C is placed on the N-type semiconductor 8153 and the P-type semiconductor 8154. The hot-end metal conductor 8152C is supported by an N-type semiconductor 8153 and a P-type semiconductor 8154. The N-type semiconductor 8153 is interposed between the hot-end metal conductor 8152C and a cold-end metal conductor 8151C. The P-type semiconductor 8154 is interposed between the hot-end metal conductor 8152C and another cold-end metal conductor 8151C. That is, one end of the N-type semiconductor 8153 is in contact with the corresponding cold-end metal conductor 8151C, and the other end is in contact with the corresponding part of the hot-end metal conductor 8152C. One end of the P-type semiconductor 8154 is in contact with another cold-end metal conductor 81510, and the other end is in contact with a corresponding part of the hot-end metal conductor 8152C. The N-type semiconductor 8153 is connected to the P-type semiconductor 8154 through a hot-end metal conductor 8152C.

The hot-end ceramic sheet 8152A is arranged in a circular sheet shape, and its size is roughly the same as that of the cold-end ceramic sheet 8151A. The hot-end ceramic sheet 8152A is interposed between the dielectric backing 82 and the hot-end heat transfer element 8152B. The hot end heat transfer element 8152B is sandwiched between the hot end ceramic sheet 8152A and the hot end metal conductor 8152C. The hot-end heat transfer element 8152B is arranged in a circular sheet shape, and its size is slightly smaller than that of the hot-end ceramic sheet 8152A. The size of the hot end heat transfer element 8152B is substantially the same as that of the cold end heat transfer element 8151B. The hot-end heat transfer element 8152B assembles the hot-end metal conductor 8152C on the hot-end ceramic sheet 8152A. The side of the hot-end heat transfer element 8152B away from the hot-end ceramic sheet 8152A is recessed from bottom to top to form a receiving space (not labeled) for accommodating the hot-end metal conductor 8152C. The hot end heat transfer element 8152B is made of heat transfer non-conductive material. The hot end heat transfer element 8152B can be thermally conductive silica gel. The hot-end metal conductor 8152C protrudes from the hot-end heat transfer element 8152B and is connected to one end of the N-type semiconductor 8153 and the P-type semiconductor 8154. The hot end metal conductor 8152C is made of the same material as the cold end metal conductor 8151C. Both the hot end metal conductor 8152C and the cold end metal conductor 8151C are made of copper.

The semiconductor refrigerator 815 also seals the cold-end metal conductor 8151C, N-type semiconductor 8153, and P-type semiconductor between the hot-end heat transfer element 8152B of the heat dissipation end 8152 and the cold-end thermal conduction element 8151B of the refrigeration end 8151 through the sealant 816. The hot-end metal conductor 8152C are sealed to prevent water vapor generated during heat exchange between the refrigeration end 8151 and the heat dissipation end 8152 from entering the semiconductor cooler 815 or entering between the semiconductor cooler 815 and the flexible circuit board 811 to cause the inside of the semiconductor cooler 815. And there is a short circuit between the welding pad 8155 of the semiconductor refrigerator 815 and the welding part (not shown) of the flexible circuit board 811. The semiconductor refrigerator 815 is respectively connected to the corresponding conductive traces (not labeled) of the cold-end heat conducting member 8151B through two conductive traces (not labeled) respectively connected to the positive electrode welding pad 8155A and the negative electrode welding pad 8155B of the cold-end ceramic sheet 8151A. The conductive traces (not labeled) of the cold-end heat-conducting element 8151B are in contact with one end of the two cold-end metal conductors 8151C placed on the cold-end heat-conducting element 8151B, and the other ends of the two cold-end metal conductors 8151C are respectively connected to one end of the N-type semiconductor 8153. One end of the P-type semiconductor 8154 is in contact with the other end of the N-type semiconductor 8153 and the other end of the P-type semiconductor 8154 is in contact with the hot-end metal conductor 8152C on the hot-end heat transfer element 8152B to realize the positive electrode welding of the cold-end ceramic sheet 8151A between the pad 8155A and the negative welding pad 8155B. The semiconductor refrigerator 815 welds the positive electrode welding pad 8155A of the cold end ceramic sheet 8151A to the welding portion 8113A of the main body portion 8111 of the flexible circuit board 811, and the negative electrode welding pad 8155B of the cold end ceramic sheet 8151A is connected to the main body portion 8111 of the flexible circuit board 811. The welding part 8113B is welded to realize the electrical connection with the flexible circuit board 811, and then can receive the control signal from the flexible circuit board 811.

The semiconductor refrigerator 815 is made by utilizing the Peltier effect of semiconductors. When a large amount of heat accumulates on the body surface of the patient's tumor with the insulated electrode 800′ attached, the tumor electric field therapy system (not shown) containing the insulated electrode 800′ inputs direct current to the semiconductor refrigerator 815 through the flexible circuit board 811, and the internal loop current of the device 815 flows from the positive electrode welding pad 8155A of the cold end ceramic sheet 8151A through the cold end heat conduction member 8151B to the cold end metal conductor 81510, N type, which is electrically connected to the positive electrode welding pad 8155A of the cold end ceramic sheet 8151A. Semiconductor 8153, hot-end metal conductor 8152C, P-type semiconductor 8154, cold-end metal conductor 8151C electrically connected to P-type semiconductor 8154, and cold-end heat conduction member electrically connected to negative electrode welding pad 8155B of cold-end ceramic chip 8151A Conductive trace from 8151B to negative solder pad 8155B of cold end ceramic chip 8151A.

The electrons of the P-type semiconductor 8154 of the semiconductor refrigerator 815 sequentially pass through the cold-end metal conductor 8151C in contact with the P-type semiconductor 8154, the conductive traces of the cold-end thermal conductor 81518 and the cold-end ceramic sheet 8151A, and the cold-end conductor 8151A in contact with the N-type semiconductor 8153. Terminal metal conductor 8151C, N-type semiconductor 8153, hot-end metal conductor 8152C to P-type semiconductor 8154. At the refrigeration end 8151 of the semiconductor refrigerator 815, when electrons flow from the P-type semiconductor 8154 to the N-type semiconductor 8153 through the cold-end ceramic sheet 8151A, the charge moves from a low energy level position to a high energy level position, which will absorb heat from the outside. At the heat dissipation end 8152 of the semiconductor refrigerator 815, when electrons flow from the N-type semiconductor 8153 to the P-type semiconductor 8154 through the hot-end metal conductor 8152C, the charge moves from a high-energy level position to a low-energy level position to dissipate heat. That is, when direct current is input to the semiconductor cooler 815 through the flexible circuit board 811, the temperature drop of the refrigeration end 8151 of the semiconductor cooler 815 will actively absorb heat from the flexible circuit board 811 through the cold end ceramic sheet 8151A, and when the temperature rises, heat will be dissipated through heat exchange between the hot-end ceramic sheet 8152A and the outside air. The heat absorbed by the cold-end ceramic sheet 8151A is transferred through the cold-end heat conducting member 8151B, the cold-end metal conductor 8151C, the N-type semiconductor 8153 and the P-type semiconductor 8154, the hot-end metal conductor 8152C, the hot-end heat transfer element 8152B, and the hot-end ceramic sheet 8152A to the outside of the insulated electrode 800′ to avoid low-temperature burns on the patient's body surface skin caused by heat accumulation on the patient's body surface skin where the insulated electrode 800′ is applied for a long time and continuously for tumor electric field therapy, without stopping the treatment to prevent the patient from The low-temperature scalding of the skin on the body surface enables the patient to have a longer time for tumor treatment and achieve better curative effect.

The active heat-absorbing insulated electrode 800′ using the semiconductor refrigerator 815 and the electrode using the epoxy glass cloth laminate with the same size as the semiconductor refrigerator 815 have the same applied electric field, the same electrode application position, and the same treatment time The temperature rise rate of the patient's skin surface is shown below, and the results show that the temperature rise rate of the patient's skin surface using the electrode of the epoxy glass cloth laminate is about 0.0223° C./s (the temperature test range is 36.5° C. to 39° C.), while the temperature rise rate of the patient's skin using the electrode of the application of the patient's skin surface with the insulated electrode 800′ is about 0.0108° C./s (the temperature test range is 36.5° C. to 39° C.); the temperature rise rate of the electrode using the semiconductor refrigerator 815 is faster than that of the epoxy glass. The electrode of the cloth laminate was lowered by about 51.5%.

It has been verified by the above tests that the insulated electrode 800′ of this embodiment can provide a semiconductor refrigerator 815 on the side of the main body 8111 of the flexible circuit board 811 away from the patient's body surface, and can be connected to the semiconductor refrigerator by the controller of the tumor treatment system through the flexible circuit board 811. After the refrigerator 815 is fed with direct current, the refrigeration end 8151 of the semiconductor refrigerator 815 can actively cool and absorb the tumor generated by the long-term and continuous electric field treatment on the patient's body surface and transmit it to the flexible tube through the adhesive part 84 and the dielectric element 813. The heat on the circuit board 811 is transmitted out of the insulating electrode 800′ through the heat dissipation end 8152, so as to quickly dissipate the heat from the patient's tumor body surface to achieve the goal of cooling down, and at the same time, the patient can have a relatively long treatment time. To ensure a good therapeutic effect, there is no need to reduce the alternating voltage applied to the flexible circuit board 811 or reduce the alternating voltage applied to the patient's tumor site through the dielectric element 813 to avoid low temperature on the patient's body surface where the electrodes are applied scald.

The backing 82 can also be provided with an opening (not shown) at a position corresponding to the hot-end ceramic sheet 8152A of the semiconductor cooler 815, so that the hot-end ceramic sheet 8152A of the semiconductor cooler 815 is exposed to the air, further improving the heat dissipation effect. According to the sample volume temperature test data of different human body parts corresponding to different indications, the semiconductor refrigerator 815 can be selected to be welded only on the electrode unit 810 in the higher temperature area, so as to reduce the overall weight of the insulated electrode 800′.

The present application also provides a tumor electric field therapy system 1000, which includes an insulated electrode 800′ and a controller (not shown) electrically connected to the insulated electrode 800′. The controller (not shown) detects the temperature of the sticking part 84 in contact with the patient's tumor site through the temperature sensor 814 on the flexible circuit board 811 of the insulated electrode 800′, and then determines whether the semiconductor refrigeration is supplied through the flexible circuit board 811. The device 815 inputs direct current.

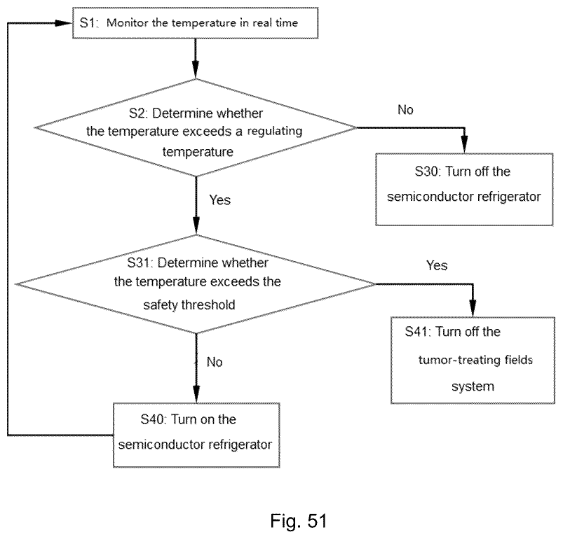

Referring to FIG. 50 and FIG. 51, the present disclosure also provides a temperature control method for a tumor electric field therapy system using the above-mentioned insulated electrode 800′. The tumor electric field therapy system includes the above-mentioned insulated electrode 800′ and a controller (not shown). The temperature control method specifically includes the following steps: step S1, real-time monitoring of the temperature of the sticker 84 of the insulating electrode 800′; step S2, judging whether the temperature obtained by monitoring exceeds the control temperature; step S3, according to the judgment, control the semiconductor refrigerator 815 to turn off or judge whether the temperature obtained by monitoring exceeds the safety threshold; step S4, control the semiconductor refrigerator 815 to turn on or control the tumor electric field therapy system to turn off according to the judgment result of whether the temperature exceeds the safety threshold in step S3.

In step S1, one side of the sticking part 84 of the insulated electrode 800′ is pasted to the skin of the patient's tumor site, and the other side is pasted to the support part 83 and the dielectric element 813. The real-time monitoring of the temperature of the sticker 84 in step S1 is specifically monitored by the temperature sensor 814 provided on the flexible circuit board 811. The temperature sensor 814 detects the temperature of the sticker 84 attached to the patient's tumor site to obtain the skin temperature of the sticker 84 attached to the patient's tumor site through the temperature sensor 814.

In step S2, judging whether the monitored temperature exceeds the regulated temperature is obtained by comparing the monitored temperature with the regulated temperature.

Judgment results in step S3 include whether the monitored temperature is lower than the regulated temperature and the monitored temperature is higher than the regulated temperature. In step S3, controlling semiconductor refrigerator 815 to close or judging whether the monitored temperature exceeds a safety threshold according to the judgment result specifically includes the following steps: step S30, controlling semiconductor refrigerator 815 to close when the monitored temperature is lower than the regulated temperature; step S31, when the monitored temperature is higher than or equal to the regulated temperature, it is judged whether the monitored temperature exceeds the safety threshold. In step S31, judging whether the monitored temperature exceeds the safety threshold is obtained by comparing the monitored temperature with the safety threshold. The difference between the safety threshold and the regulated temperature is within 4° C., so as to avoid human discomfort caused by too low temperature. Preferably, the regulating temperature is 39°, and the safety threshold is 41°.

The judgment result of step S4 based on whether the safety threshold is exceeded in step S3 includes the monitored temperature exceeding or equal to the safety threshold and the monitored temperature being lower than the safety threshold. In step S4, controlling the semiconductor refrigerator 815 to be turned on or controlling the tumor treatment system to be turned off according to the judgment result of step S3 exceeding the safety threshold specifically includes the following steps: Step S40, when the monitored temperature is lower than the safety threshold, controlling the semiconductor refrigerator 815 to be turned on and repeat the cycle of steps S1 to S3; step S41, when the monitored temperature is higher than or equal to the safety threshold, control the tumor electric field therapy system to shut down.

Controlling the semiconductor cooler 815 to turn on in step S4 is specifically realized by controlling the flexible circuit board 811 to input direct current to the semiconductor cooler 815. Controlling the semiconductor cooler 815 to turn on in step S40 refers to inputting direct current to the semiconductor cooler 815 through the flexible circuit board 811 so that the semiconductor cooler 815 is in an open state. Controlling the tumor electric field therapy system to turn off in step S41 refers to controlling the tumor electric field therapy system to be in an off state, which is specifically realized by turning off the power supply of the tumor electric field therapy system.

Controlling the semiconductor refrigerator 815 to be in an open state is realized by inputting direct current to the semiconductor refrigerator 815. Controlling the semiconductor refrigerator 815 to be in an off state is realized by turning off the direct current input to the semiconductor refrigerator 815.

When the semiconductor refrigerator 815 is in the open state, the refrigeration end 8151 of the semiconductor refrigerator 815 can actively absorb the heat generated by the body surface of the tumor part of the patient transmitted to the flexible circuit board 811 through the adhesive part 84 and the dielectric element 813, as well as the heat generated on the insulated electrode 800′. During operation, the heat generated by the conductive plate (not shown) of the flexible circuit board 811 and the dielectric element 813 is quickly dissipated through the heat dissipation end 8152 of the semiconductor refrigerator 815, thereby quickly reducing the sticking part 84 of the insulated electrode 800′ temperature, thereby reducing the body surface temperature of the patient's tumor site, without reducing the alternating current applied to the insulated electrode 800′ or reducing the alternating current applied to the dielectric element 813 through the conductive pad (not shown) of the flexible circuit board 811. The purpose of reducing the body surface temperature of the patient's tumor by changing the electric field can realize long-term and continuous tumor electric field therapy and improve the therapeutic effect.

Under normal circumstances, after the semiconductor refrigerator 815 is turned on, the skin surface temperature will drop slowly, but due to some internal or external reasons, there will be a small probability that the electric field will be out of control. When the detected temperature exceeds the upper safe temperature limit (for example, 41° C.), the power supply of the tumor electric field therapy system will be turned off. The tumor electric field therapy system needs to be turned on manually for it to work again.

With such a setting, the temperature gradient (39° C., 41° C.) can be controlled to keep the tumor electric field therapy system running as much as possible while reducing the skin surface temperature. Compared with the traditional method of directly turning off the tumor electric field therapy system for cooling, the temperature control method can have more time to implement the alternating electric field treatment and improve the treatment effect.

It should be noted that in this disclosure, relational terms such as “first” and “second” are only used to distinguish one entity or operation from another entity or operation, and do not necessarily require or imply these No such actual relationship or order exists between entities or operations. The terms “comprises”, “comprises” or any other variation thereof are intended to cover a non-exclusive inclusion such that a process, method, article or apparatus comprising a set of elements includes not only those elements but also others not expressly listed elements, or also elements inherent in such a process, method, article, or apparatus. Without further limitations, an element defined by the phrase “comprising a . . . ” does not exclude the presence of additional identical elements in the process, method, article or apparatus comprising said element.

The methods and devices provided by the embodiment of the present disclosure have been described in detail above. The principles and implementation methods of the present disclosure have been explained by using specific examples in this paper. The descriptions of the above embodiments are only used to help understand the methods and methods of the present disclosure. core idea; at the same time, for those of ordinary skill in the art, according to the idea of this application, there will be changes in the specific implementation and application scope. In summary, the content of this specification should not be construed as limiting the application.

The disclosure of this patent document contains material that is protected by copyright. This copyright belongs to the copyright owner. The copyright owner has no objection to the reproduction by anyone of the patent document or the patent disclosure as it exists in the official records and files of the Patent and Trademark Office.

1. A tumor electric field therapy system comprising an electric field therapy device having a plurality of preset system parameters and at least two pairs of insulated electrodes electrically connected to the electric field therapy device, wherein the electric field therapy device includes a MCU control unit with a reference voltage, a direction control unit connected to the MCU control unit, a DC power control unit electrically connected to the MCU control unit, and an AC voltage control unit both electrically connected with the direction control unit and the MCU control unit, and wherein the MCU control unit is configured to control the AC voltage control unit to generate an alternating electrical signal with an AC voltage according to the system parameters of the electric field therapy device, and wherein the MCU control unit is also configured to drive the direction control unit to cyclically and alternately apply the alternating electrical signal received by the AC voltage control unit to different pairs of insulated electrodes, so as to realize the switching of the alternating electrical signal between different pairs of insulated electrodes, and wherein the system parameters of the electric field therapy device include an output AC voltage amplitude of the alternating electrical signal stored therein, and wherein the alternating electrical signal applied to each pair of the insulated electrodes has a continuous on-time period during each respective working cycles, and wherein each of the continuous on-time of the alternating electrical signal includes an initial switching-on time period t3, an intermediate on-time period and a final switching-off time period t4; and wherein the alternating electrical signal has a specific voltage during the intermediate on-time period and the specific voltage is equal to or less than a peak of the output AC voltage amplitude of the alternating electrical signal stored in the electric field therapy device; and wherein during the switching-on time period t3, the MCU control unit controls the DC power control unit to increase the AC voltage value of the alternating electrical signal applied from the AC voltage control unit to the insulated electrodes arranged in pairs from 0 to the specific voltage at a constant speed and a voltage change of the alternating electrical signal applied by the AC voltage control unit per millisecond is a set to be a constant value; and wherein during the switching-off time period t4, the MCU control unit controls the DC power supply control unit to decrease the AC voltage value of the alternating electrical signal applied from the AC voltage control unit to the insulated electrode arranged in pairs from the specific voltage to 0 at a constant speed and a voltage change of the alternating electrical signal applied by the AC voltage control unit per millisecond is set to be a constant value.

2. The tumor electric field therapy system according to claim 1, wherein the ratio of the voltage change per millisecond of the alternating electrical signal applied by the AC voltage control unit to the specific voltage is less than 5%.

3. The tumor electric field therapy system according to claim 1, wherein the electric field therapy device further includes an inverter boost control unit both connected to the MCU control unit and the DC power supply control unit and a filter control unit connected to the inverter boost control unit, and wherein the system parameters of the electric field therapy device include an electric field frequency and a direction switching period of the alternating electrical signal, and wherein the MCU control unit generates a pulse signal transmitted to the inverter boost control unit according to the reference voltage thereof, the electric field frequency of the alternating electrical signal stored in the electric field therapy device and the output AC voltage amplitude of the alternating electrical signal stored in the electric field therapy device.

4. The tumor electric field therapy system according to claim 3, wherein the MCU control unit drives the direction control unit to cyclically and alternately switch the alternating electrical signal applied to different pairs of the insulated electrodes through the AC voltage control unit according to the direction switching period of the alternating electrical signal of the electric field therapy device.

5. The tumor electric field therapy system according to claim 3, wherein the connection and disconnection between the MCU control unit and the DC power supply control unit and whether the pulse signal is applied to the inverter boost control unit or not are controlled by the MCU control unit according to the direction switching period of the alternating electrical signal of the electric field therapy device.

6. The tumor electric field therapy system according to claim 5, wherein the direction control unit is configured to be switched after the communication between the MCU control unit and the DC power supply control unit is disconnected and the pulse signal transmitted from the MCU control unit to the inverter boost control unit is stopped.

7. The tumor electric field therapy system according to claim 6, wherein after the direction control unit is switched, the MCU control unit activates the DC power control unit, controls the DC power control unit to output a DC signal to the inverter boost control unit and also outputs a pulse signal to the inverter boost control unit.

8. The tumor electric field therapy system according to claim 1, wherein the AC voltage of the alternating electrical signal is increased from 0 to the specific voltage at a constant speed with a voltage change of 4V per millisecond during the switching-on time period t3.

9. The tumor electric field therapy system according to claim 1, wherein the switching-on time period t3 is determined as follows: t3=V/ΔV*t, wherein V is the specific voltage, t is 1 ms, and ΔV is the voltage change of the alternating electrical signal outputted by the AC voltage control unit per millisecond.

10. The tumor electric field therapy system according to claim 1, wherein the MCU control unit includes a digital-to-analog conversion module with a DAC data register, and wherein the MCU control unit calculates a voltage output increment of the digital-to-analog conversion module per millisecond based on the reference voltage thereof, the voltage change of the alternating electrical signal outputted by the AC voltage control unit per millisecond, the specific voltage, and the DAC data register value corresponding to the specific voltage.

11. The tumor electric field therapy system according to claim 10, wherein the MCU control unit calculates a voltage output decrement of the digital-to-analog conversion module per millisecond based on the reference voltage thereof, the voltage change of the alternating electrical signal outputted by the AC voltage control unit per millisecond, the specific voltage, and the DAC data register value corresponding to the specific voltage.

12. The tumor electric field therapy system according to claim 11, wherein the voltage output increment or decrement per millisecond of the digital-to-analog conversion module is determined by the following method: ΔV_{DAC}=(3.3*1000*ΔV*DAC)/(4096*V), wherein ΔV_{DAC} is the voltage output increment or decrement of the digital-to-analog conversion module per millisecond and measured in millivolts; and the reference voltage of the MCU control unit is 3.3V, and the DAC data register corresponding to the MCU reference voltage value is 4096 and equal to 2¹²; ΔV is the voltage change of the alternating electrical signal outputted by the AC voltage control unit per millisecond and measured in volts; V is the specific voltage and measured in volts; DAC is the DAC data register value of the specific voltage in the DAC data register.

13. The tumor electric field therapy system according to claim 1, wherein the switching-off time period t4 is obtained by the following method: t4=V/ΔV*t, wherein Vis the specific voltage, t is 1 ms, and ΔV is the voltage change of the alternating electrical signal outputted by the AC voltage control unit per millisecond.

14. The tumor electric field therapy system according to claim 1, wherein the AC voltage of the alternating electrical signal is decreased from the specific voltage to 0 at a constant speed with a voltage change of 4V per millisecond during the switching-off time period t4.

15. The tumor electric field therapy system according to claim 3, wherein the electric field therapy device further includes a first direction switch that is electrically connected to the direction control unit and controls the connection and disconnection between the AC voltage control unit and one pair of insulated electrodes and a second direction switch that is electrically connected to the direction control unit and controls the connection and disconnection between the AC voltage control unit and another pair of insulated electrodes.

16. The tumor electric field therapy system according to claim 15, wherein the MCU control unit includes a storage module, an execution module communicatively connected with the storage module, a digital-to-analog conversion module (DAC) communicatively connected with the execution module, and a control module that controls the storage module, the execution module and the digital-to-analog conversion module to perform corresponding operations, and wherein the storage module of the MCU control unit, the execution module of the MCU control unit, the digital-to-analog conversion module of the MCU control unit, the control module of the MCU control unit, the DC power supply control unit, the inverter boost control unit and the AC voltage control unit jointly form an AC signal generator, and wherein the storage module of the MCU control unit, the execution module of the MCU control unit, the control module of the MCU control unit, the direction control unit, the first direction switch and the second direction switch both electrically connected to the direction control unit jointly form an AC signal controller.

17. The tumor electric field therapy system according to claim 16, wherein at least two pairs of the insulated electrodes include a first pair and a second pair of insulated electrodes disposed on the patient's torso surface; and wherein the AC signal controller is configured to generate periodic control signals each having a first output state with a duration between 500 ms and 980 ms and a second output state with a duration between 500 ms and 980 ms, and wherein the AC signal generator generates a first AC signal applied to the first pair of insulated electrodes when the control signal is in the first output state and a second AC signal applied to the second pair of insulated electrodes when the control signal is in the second output state, and wherein the switch between the first AC signal generated between the first pair of insulated electrodes and the second AC signal generated between the second pair of insulated electrodes is achieved through the switch between the first output state and the second output state.

18. The tumor electric field therapy system according to claim 17, wherein the first output state has the duration with a first time period T1, and wherein the second output state has a duration with a second time period T2, and wherein the first time period T1 and the second time period T2 are same with each other.

19. The tumor electric field therapy system according to claim 18, wherein the first time period T1 and the second time period T2 are both 50% of respective operating period.

20. The tumor electric field therapy system according to claim 18, wherein the first AC signal has an increasing AC voltage amplitude during the switching-on time period t3 and a decreasing AC voltage amplitude in the switching-off time period t4 in each of the first time periods T1; and wherein the second AC signal has an increasing AC voltage amplitude during the switching-on time period t3 and a decreasing AC voltage amplitude during the switching-off time period t4 in each of the second time period T2.

21. The tumor electric field therapy system according to claim 20, wherein the durations of the switching-on time period t3 and the switching-off time period t4 are both less than 10% of the duration of the first time period T1 or the second time period T2.

22-191. (canceled)
